(19) United States
(12) Patent Application Publication
(10) Pub. No.: US 2011/0065196 A1
(43) Pub. Date: Mar. 17, 2011

(54) Trityl Derivatives for Enhancing Mass Spectrometry

(76) Inventors: Mikhail Shchepinov, Oxford (GB); Pablo Bernad, Oxford (GB)

(21) Appl. No.: 11/922,235

(22) PCT Filed: Jun. 16, 2006
(86) PCT No.: PCT/GB2006/002208

§ 371 Date: Jul. 23, 2009

(30) Foreign Application Priority Data
Jun. 16, 2005 (GB) 0512316.1

(51) Int. Cl. G01N 33/50; C07K 1/13; C07C 43/205; C07C 323/16; C07C 323/60; C07C 235/34; C07H 1/00; C07H 21/04

(52) U.S. Cl. 436/86; 530/410; 568/640; 568/49; 564/162; 564/171; 536/123.1; 536/23.1; 530/408; 436/94; 436/128; 436/120; 436/106

(57) ABSTRACT

The present invention provides a method of forming an ion of formula (I) comprising the steps of: (i) reacting a compound of the formula (IIa); with a biopolymer, B_{P}, having at least one group capable of reacting with M to form a covalent linkage, to provide a biopolymer derivative of the formula (IIIa); and (ii) cleaving the C—X bond between X and the α-carbon atom of the derivative of formula (IIIa) to form the ion of formula (I); where: (IV) is a carbon atom bearing a single positive charge or a single negative charge; and X is a group comprising a thioether sulphur atom bound directly to the α-carbon which is capable of being cleaved from the α-carbon atom to form an ion of formula (I). The biopolymer derivatives of the invention have enhanced ionisability with respect to free biopolymer (B_{P}) enabling improved analysis of the biopolymer using mass spectrometry.

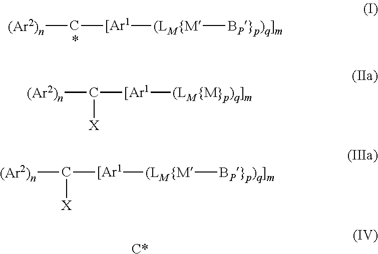

All documents cited herein are incorporated by reference in their entirety.

### TECHNICAL FIELD

This invention relates to derivatised biopolymers and ions obtainable therefrom. The invention further relates to compounds and solid supports useful for producing the derivatised biopolymers and ions of the invention.

### BACKGROUND OF THE INVENTION

Mass spectrometry is a versatile analytical technique possessing excellent detection range and speed of detection with respect to High Performance Liquid Chromatography (HPLC), Gas Chromatography (GC), Infra-Red (IR) and Nuclear Magnetic Resonance (NMR).

However, many biopolymers, such as carbohydrates and proteins, are difficult to analyse using mass spectrometry due to significant difficulties in ionising the biopolymer, even using Matrix Assisted Laser Desorption/Ionisation Time Of Flight (MALDI-TOF) techniques. Despite the considerable resolving power of 2D-PAGE, this technology has fallen far short of the ultimate goal of displaying the whole proteome in a single experiment, as many proteins are resistance to 2D-PAGE analysis (e.g those with low or high molecular masses, membrane proteins, proteins with extreme isoelectric points, etc.). Many proteins are thus invisible to 2-D PAGE [Cravat & Sørensen (2000) Current Opinion in Chemical Biology vol. 4, p. 663-668].

International patent application no. PCT/GB2004/005140 discloses the covalent attachment of a biopolymer to a triarylmethyl derivative via an aromatic group adjacent to the central α-triarylmethyl carbon atom. The biopolymer-bound triarylmethyl derivatives have improved ionisability with respect to free biopolymer and allow for improved analysis of the biopolymer by mass spectrometry.

The compounds disclosed in PCT/GB2004/005140 typically employ a group comprising an ether oxygen atom bound directly to the α-triarylmethyl carbon atom capable of being cleaved from the carbon atom to form an ion. Examples of such groups include hydrocarbyloxy groups (e.g. ethoxy). However, the bond between the ether oxygen and the α-triarylmethyl carbon atom is often extremely sensitive to acidic conditions, which may be inconvenient when attaching a biopolymer to the triarylmethyl derivative by causing premature release of the leaving group prior to mass spectrometry analysis.

There is therefore a need for improvements in triarylmethyl derivatives for assisting the analysis of biopolymers by mass spectrometry.

### DISCLOSURE OF THE INVENTION

It has now been found that triarylmethyl derivatives having a thioether sulphur atom in place of the ether oxygen atom allow improvements in the analysis of biopolymers by mass spectrometry.

In particular, it has been discovered that the thioether-containing compounds of formula (IIa) below are more stable to acidic conditions compared with compounds employing a group comprising an ether oxygen atom bound directly to the α-triarylmethyl carbon atom. Advantageously, attachment of the biopolymer to the compounds of formula (IIa) may therefore be effected under acidic conditions without premature release of the leaving group prior to mass spectrometry analysis.

Furthermore, it has surprisingly been discovered that the compounds of formula (IIIa) below have improved ionisability, especially in LDI techniques absent a matrix, compared with compounds employing a group comprising an ether oxygen atom bound directly to the α-triarylmethyl carbon atom. Furthermore, the compounds of formula (IIIa), especially in LDI techniques absent a matrix, provide cleaner mass spectra allowing for superior analysis of the biopolymer.

The invention provides methods of forming ions from covalent or ionic compounds and solid supports.

### Derivatised Biopolymers

The invention provides a method of forming an ion of formula (I):

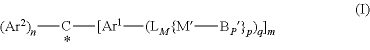

comprising the steps of:
(i) reacting a compound of the formula (IIa):

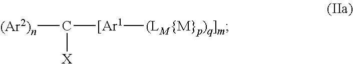

with a biopolymer, B_{P}, having at least one group capable of reacting with M to form a covalent linkage, to provide a biopolymer derivative of the formula (IIIa):

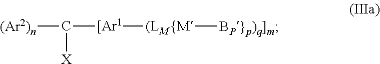

and
(ii) cleaving the C—X bond between X and the α-carbon atom of the derivative of formula (IIIa) to form the ion of formula (I);
   where:
C★ is a carbon atom bearing a single positive charge or a single negative charge;
X is a group comprising a thioether sulphur atom bound directly to the α-carbon which is capable of being cleaved from the α-carbon atom to form an ion of formula (I);
M is independently a group capable of reacting with B_{P} to form the covalent linkage;
B_{P}′ is independently the biopolymer residue of B_{P} produced on formation of the covalent linkage;
M′ is independently the residue of M produced on formation of the covalent linkage;
Ar¹ is independently an aromatic group or an aromatic group substituted with one or more A;
Ar² is independently an aromatic group or an aromatic group substituted with one or more A;
   optionally wherein (a) two or three of the groups Ar¹ and Ar² are linked together by one or more L⁵, where L⁵ is independently a single bond or a linker atom or group; and/or (b) two or three of the groups Ar¹ and Ar² together form an aromatic group or an aromatic group substituted with one or more A;
A is independently a substituent;
L_{M} is independently a single bond or a linker atom or group;
n=0, 1 or 2 and m=1, 2, or 3, provided the sum of n+m=3;
p independently=1 or more; and
q independently=1 or more.
The compounds of formula (IIa) may optionally be purified after step (i).

The invention also provides biopolymer derivatives of the formula (IIIa), as defined above. The biopolymer derivatives of the invention have enhanced ionisability with respect to free biopolymer, B_{P}. Advantageously, the biopolymer derivatives may not require a matrix (e.g. as used in MALDI-MS) in order to elicit ionisation, although a matrix may help to enhance ionisation. Preferably, ionisation may be obtained without requiring acid treatment, in particular by direct laser illumination. Moreover, it has been discovered that the compounds of formula (IIIa) below have improved ionisability, especially in LDI techniques absent a matrix, compared with compounds employing a group comprising an ether oxygen bound directly to the α-triarylmethyl carbon atom. Furthermore, the compounds of formula (IIIa), especially in LDI techniques absent a matrix, provide cleaner, i.e. less cluttered, mass spectra allowing for superior analysis of the biopolymer.

The ions of formula (I) are stabilised by the resonance effect of the aromatic groups Ar¹ and Ar². Electron-withdrawing groups, when C★ is an anion, or electron-donating groups, when C★ is a cation, may optionally be provided on Ar¹ and/or Ar² to assist this resonance effect. Consequently, the biopolymer derivatives of the invention readily form ions of formula (I) relative to the native biopolymer, B_{P}.

The ions of formula (I) are generally only ever seen on a mass spectrum with a single charge, which is advantageous since it reduces cluttering of the mass spectrum.

The invention also provides compounds of the formula (IIa), as defined above. Compounds of formula (IIa) are more stable to acidic conditions compared with compounds employing a group comprising an ether oxygen bound directly to the α-triarylmethyl carbon atom. Advantageously, attachment of the biopolymer may therefore be effected under acidic conditions without premature release of the leaving group prior to mass spectrometry analysis. Compounds of formula (IIa) are useful for forming ions of formula (I). As the difference in the molecular mass of the ions of formula (I) and that of the free biopolymer can be accurately calculated, the derivatised compounds of the invention allow analysis of the biopolymer B_{P}, which may be otherwise difficult or impossible to analyse using known mass spectrometrical techniques.

Other advantageous features of the compounds of the invention include more uniformity of the signal intensity between different analytes (useful for quantitative studies) and similar desorption properties between compounds with different, but close, masses, so that techniques such as isotope coded affinity tagging (ICAT) can be employed with the compounds of the invention.

The homogeneous methods of the invention are particularly appropriate for small molecules, e.g. amines.

### Solid Supports

The ions of formula (I) may also be formed using a derivatised solid support.

The invention therefore provides a method of forming an ion of formula (I) comprising the steps of:
(i) reacting a solid support of formula (IVai):

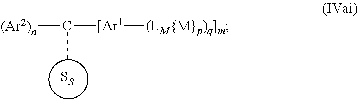

with a biopolymer, B_{P}, having at least one group capable of reacting with M to form a covalent linkage, to provide a modified solid support of the formula (Vai):

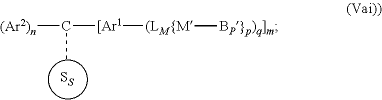

and
(iia) cleaving the C—S_{S} bond between the α-carbon atom of the modified solid support of formula (Vai) and the solid support S_{S} to form the ion of formula (I);
   where:

Ar¹, Ar², B_{P}′, L_{M}, M, M′, n, m, p and q are as defined above;

S_{S} is a solid support; and

C—S_{S} comprises a cleavable bond between C and S_{S} involving a thioether sulphur atom bound directly to the α-carbon atom.

The invention also provides a method of forming an ion of formula (I) comprising the steps of:
(i) reacting a solid support of formula (IVaii), or (IVaiii):

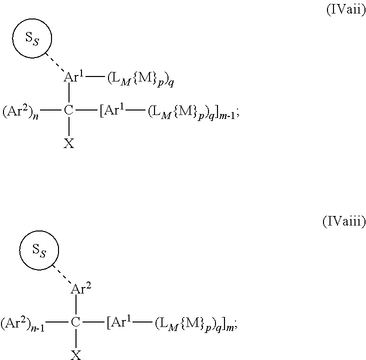

with a biopolymer, B_{P}, having at least one group capable of reacting with M to form a covalent linkage, to provide a modified solid support of the formula (Vaii), or (Vaiii), respectively:

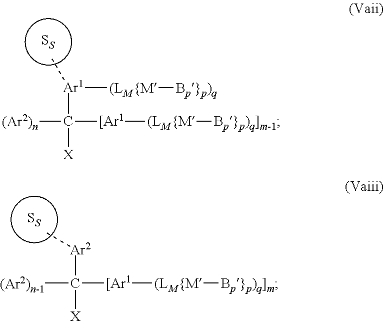

and either:
(iib) for modified solid supports of formula (Vaii), either simultaneously or sequentially, cleaving the C—X bond between X and the α-carbon atom and cleaving the S_{S}—Ar₁ bond between the solid support and the Ar¹ group to form the ion of formula (I); or
(iic) for modified solid supports of formula (Vaiii), either simultaneously or sequentially, cleaving the C—X bond between X and the α-carbon atom and cleaving the S_{S}—Ar² bond between the solid support and the Ar² group to form the ion of formula (I);
   where:
X, Ar¹, Ar², B_{P}′, L_{M}, M, M′, n, m, p and q are as defined above;
S_{S} is a solid support;
S_{S}—Ar¹ comprises a cleavable bond between Ar¹ and S_{S}; and
S_{S}—Ar² comprises a cleavable bond between Ar² and S_{S}.

The cleavable bond of C—S_{S}, S_{S}—Ar¹ or S_{S}—Ar² may be a covalent, ionic, hydrogen, dipole-dipole or van der Waals bond.

The invention further provides a method of forming an ion of formula (I) comprising the steps of:
(i) reacting a solid support of formula (IVaiv):

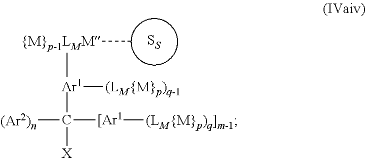

with a biopolymer, B_{P}, having at least one group capable of reacting with M to form a covalent linkage, to provide a modified solid support of the formula (Vaiv):

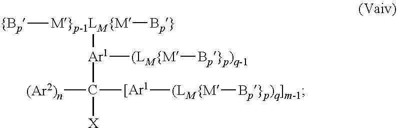

and
(iia) cleaving the C—X bond between X and the α-carbon atom to form the ion of formula (I);
   where:
X, Ar¹, Ar², B_{P}′, L_{M}, M, M′, p, q, n, m, and S_{S} are as defined above;
M″—S_{S} comprises a bond between M″ and S_{S}; and
M″ is the same as M except that S_{S} is bound to a portion of M which does not form part of M′.

In this embodiment of the invention, the solid support is bound to a part of group M″ which does not go on to form the residue M′. Thus, the derivatised biopolymer will be released from the solid support during the derivativisation step and an additional step of cleaving the biopolymer from the solid support is not required.

The modified solid supports of formulae (Vai), (Vaii) (Vaiii), or (Vaiv) may optionally be washed after step (i).

The invention also provides solid supports of the formulae (IVai), (IVaii), (IVaiii) and (IVaiv) as defined above. Similarly, the invention provides modified solid supports of the formulae (Vai), (Vaii), (Vaiii) and (Vaiv) as defined above.

The heterogeneous methods of the invention are particularly appropriate for synthetic biopolymers, e.g. oligonucleotides, peptides and carbohydrates.

### Methods of Analysis

The invention also provides a method for analysing a biopolymer, B_{P}, comprising the steps of:
(i) reacting the biopolymer B_{P} with a compound of formula (IIa) or a solid support of formula (IVai), (IVaii), (IVaiii) or (IVaiv);
(ii) providing an ion of formula (I); and
(iii) analysing the ion of formula (I) by mass spectrometry.

The biopolymer will typically have been obtained using a preparative or analytical process. For example, it may have been purified using various separation methods (e.g. 1-dimensional or 2-dimensional, reverse-phase or normal-phase separation, by e.g. chromatography or electrophoresis) and the separation may be based on any of a number of characteristics (e.g. isoelectric point, molecular weight, charge, hydrophobicity, etc.). Typical methods include 2D SDS-PAGE, 2D liquid chromatography (e.g. Multidimensional Protein Identification Technology, MudPIT, or 2D HPLC methods). The separation method can preferably interface directly with the mass spectrometer.

Known analytical techniques can thus be adapted or improved by the method of the invention. A particularly preferred method involves 2D-PAGE of a biopolymer, or mixture of biopolymers, selection of a spot of interest in the electrophoretogram, and then derivatisation and analysis of that spot using the techniques of the invention. The biopolymer may be proteolytically digested prior to its analysis (typically within the PAGE gel, but optionally digested after extraction from the gel) and/or may itself be the product of a proteolytic digest.

The invention also provides, in a method for analysing a biopolymer, B_{P}, the improvement consisting of: (i) reacting a biopolymer, B_{P} with a compound of formula (IIa) or a solid support of formula (IVai), (IVaii), (IVaiii) or (IVaiv); (ii) providing an ion of formula (I); and (iii) analysing the ion by mass spectrometry.

Typically, the analysis by mass spectrometry is carried out in a spectrometer which is suitable for MALDI-TOF spectrometry.

In the spectrometer, the ion source may be a matrix-assisted laser desorption ionisation (MALDI), an electrospray ionisation (ESI) ion source, a Fast-Atom Bombardment (FAB) ion source. Preferably, the ion source is a MALDI ion source. The MALDI ion source may be traditional MALDI source (under vacuum) or may be an atmospheric pressure MALDI (AP-MALDI) source. MALDI is a preferred ionisation method, although the use of a matrix is generally not required

In the spectrometer, the mass analyser may be a time of flight (TOF), quadrupole time of flight (Q-TOF), ion trap (IT), quadrupole ion trap (Q-1T), triple quadrupole (QQQ) Ion Trap or Time-Of-Flight Time-Of-Flight (TOFTOF) or Fourier transform ion cyclotron resonance (FlICR) mass analyser. Preferably, the mass analyser is a TOF mass analyser.

Preferably, the mass spectrometer is a MALDI-TOF mass spectrometer.

### Further Embodiments

### M′ Bound to B_{P}′ by a Non-Covalent Linker

The above-mentioned embodiments of the invention may also be provided in which M′ is bound to B_{P}′ by a non-covalent bond. All the other features of the invention are the same except the groups which relate to the non-covalent bond between M′ and B_{P}′.

The non-covalent bond may be direct between M′ and B_{P}′ or may be provided by one or more binding groups present on M′ and/or B_{P}′.

Preferred non-covalent bonds are those having an association constant (IQ of at least 10¹⁴ M⁻¹, preferably about 10¹⁵ M⁻¹.

In preferred embodiment, one of M and B_{P}′ will have a binding group comprising biotin, and the other of M′ and B_{P}′ will have a binding group comprising avidin or streptavidin.

Preferably, when the compounds of the invention comprise a non-covalent bond between M′ and B_{P}′ and a cleavable bond between C and S_{S}, Ar¹ and S_{S}, or Ar² and S_{S}, these bonds are differentially cleavable. More preferably, the non-covalent bond between M′ and B_{P}′ is not cleaved under conditions which the cleavable bond between C and S_{S}, Ar¹ and S_{S}, or Ar² and S_{S}, as appropriate, is cleaved.

L_{M} Bound to Ar¹ by More than One Bond

The above-mentioned embodiments of the invention may also be provided in which L_{M} is bound to Ar¹ by more than one covalent bond (e.g. 2 or 3 bonds) which are either single, double or triple covalent bonds, or one or more multiple bonds (e.g. double or triple covalent bonds). All the other features of the invention are the same except the groups which relate to the bond or bonds between Ar¹ and L_{M}.

Ionisation of Compounds Other than Biopolymers

In addition to biopolymers, the present invention may be used for ionising any molecule or complex of molecules which requires mass spectrum analysis. Thus, the above-mentioned embodiments of the invention may also be provided in which B_{P} is replaced by any molecule or complex having at least one group capable of reacting with M to form a covalent linkage. All the other features of the invention are the same, except group M is group capable of reacting with the molecule to be analysed.

Examples of other molecules which may be analysed in the present invention include non-biological polymers (e.g. synthetic polyesters, polyamides and polycarbonates), petrochemicals and small molecules (e.g alkanes, alkenes, amines, alcohols, esters and amides). Amines are particularly preferred.

Examples of complexes which may be analysed in the present invention include double- and triple-stranded RNA, DNA and/or peptide nucleic acid (PNA) complexes, enzyme/substrate complexes, multimeric proteins (e.g. dimers, trimers, tetramers, pentamers, etc.), virions, etc.

### Disclaimers

Preferably, all embodiments of the invention (including products of formulae (IIa)) involving or relating to compounds wherein X is —S-succinimidyl or a nucleic acid comprising a thioether sulphur atom bound directly to the α-carbon (e.g. —S-oligonucleotide) are disclaimed.

### Preferred Embodiments

### Definition of C★

Preferably, C★ bears a single positive charge such that ions of the invention are cations and the ion of formula (I) has the following structure:

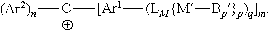

n, m, p and q

For the purposes of compounds of the invention having n−1 groups Ar², n may not be less than 1.

Preferably n=2 and m=1.

Preferably p=1, 2 or 3. Preferably p=1.

Preferably q=1, 2 or 3. Preferably q=1.

Preferably n=2, m=1, p=1 and q=1. The ion of formula (I) thus has the structure:

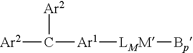

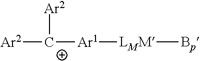

or more preferably
and the compounds of formulae (IIa), (IIIa), (IVai), (IVaii), (IVaiii), (IVaiv), (Vai), (Vaii), (Vaiii) and (Vaiv) have the structures disclosed in table 1.

### Biopolymers

The term ‘biopolymer’ includes polymers found in biological samples, including polypeptides, polysaccharides, and polynucleotides (e.g. DNA or RNA). Polypeptides may be simple copolymers of amino acids, or they may include post-translational modifications e.g. glycosylation, lipidation, phosphorylation, etc. Polynucleotides may be single-stranded (in whole or in part), double-stranded (in whole or in part), DNA/RNA hybrids, etc. RNA may be mRNA, rRNA or tRNA.

Advantageous biopolymers are those which do not readily form a molecular ion in known MALDI-TOF MS techniques, especially those which do not form a molecular ion on illumination of laser light at 340 nm.

Biopolymers for use in the invention comprise two or more monomers, which may be the same or different as each other. Preferred biopolymers comprise at least pp monomers, where pp is 5 or more (e.g. 6, 7, 8, 9, 10, 15, 20, 25, 30, 35, 40, 45, 50, 60, 70, 80, 90, 100, 125, 150, 175, 200, 250). More preferred biopolymers comprise ppp or fewer monomers where ppp is 300 or less (e.g. 200, 100, 50).

Biopolymers may have a molecular mass of at least qq kDa, where qq=0.5 or more (e.g. 0.6, 0.7, 0.8, 0.9, 1, 1.5, 2, 3, 4, 5, 6, 7, 8, 9, 10, 15, 20, 25, 30, 40, 50, 75, 100, etc.). Preferred biopolymers are those having a molecular mass within the range of detection of a mass spectrometer. More preferred biopolymers have a molecular mass of qqq kDa or less, where qqq is 30 or less (e.g. 20, 10, 5).

Preferably, the mass, m(IX), of the fragment (IX)

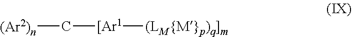

of the cation of formula (I) is significantly less than the mass, m(B_{P}′), of the biopolymer residue B_{P}′. For example the ratio m(B_{P}′)/m(IX) is preferably more than nn, where nn is at least 2 (e.g. 3, 4, 5, 10, 100, 1000, etc.).

The invention is suitable for use with purified biopolymers or mixtures of biopolymers. For example, a pure recombinant protein could be derivatised and analysed by MS, or biopolymers within a cellular lysate or extract could be derivatives and then analysed.

Preferred biopolymers are polypeptides. Particularly preferred biopolymers are polypeptides formed after proteolytic digestion of a protein.

### Biopolymers Bound to Solid Supports

In preferred embodiments of the invention the biopolymer is bound to a solid support such that it is cleavable from the solid support at least once it has been derivatised by a compound of the invention. B_{P} is thus derivatised in situ while bound to the support, and is then released. As the biopolymer is bound to the solid support, this aspect of the invention is particular relevant to methods involving compounds of formulae (IIa).

The biopolymer may be bound to the solid support by a covalent, ionic, hydrogen, dipole-dipole or van der Waals bond (also known as a dispersion bond or a London forces bond). The covalent, ionic, hydrogen, dipole-dipole or van der Waals bond may be direct between the biopolymer and the solid support or may be provided by one or more binding groups present on the biopolymer and/or solid support. Preferred groups are non-covalent groups.

Examples of groups which can form these types of bond, and methods for cleaving these types of bond, are set out below in connection with C—S_{S} bonds, etc.

In a particularly preferred embodiment, the solid support is provided with —(NMe_{S})⁺ binding groups and the biopolymer has a net negative charge, or vice versa (i.e. the —(NMe₃)⁺ is on the biopolymer).

In other preferred embodiments, the solid support is provided with anions such as carboxylate, phosphate or sulphate, or anions formed from acid groups, and the biopolymer (e.g. a histone) has a net positive charge, or vice versa.

Reactivity with Group M

The biopolymers have at least one reactive group capable of reacting with M to form a covalent linkage. Such groups typically include naturally occurring groups and groups formed synthetically on the biopolymer.

Naturally occurring groups include lipid groups of lipoproteins (e.g. myristoyl, glycosylphosphatidylinositol, ethanolamine phosphoglycerol, palmitate, stearate, S- or N- or O-acyl groups, lipoic acid, isoprenyl, geranylgeranyl, farnesyl, etc.), amide, carbohydrate groups of N- and O-glycoproteins, amine groups (e.g. on lysine residues or at the N-terminus of a protein), hydroxyl (e.g. in β-hydroxyaspartate, (3-hydroxyasparagine, 5-hydroxylysine, ¾-hydroxyproline), thiol, sulfhydryl, phosphoryl, sulfate, methyl, acetyl, formyl (e.g. on N-terminal methionines from prokaryotes), phenyl, indolyl, guanidyl, hydroxyl, phosphate, methylthio, ADP-ribosyl etc.

The reactive group is bound to the biopolymer by one or more covalent bonds (e.g. 2 or 3 bonds), which are either single, double or triple covalent bonds (preferably single bonds). Preferably, the reactive group is bound to the biopolymer by one single bond.

Groups which may be formed naturally or synthetically on the biopolymer and which are bound to the biopolymer by one bond include: —NR₂ e.g. —NHR, especially —NH₂; —SR e.g. —SH; —OR e.g. —OH; —B(R)Y; —BY₂; —C(R)₂Y; —C(R)Y₂; —CY₃; —C(═Z)Y e.g. —C(═O)Y; —Z—C(═Z)Y; —C(═Z)R e.g. —C(═Z)H, especially —C(═O)H; —C(R)(OH)OR; —C(R)(OR)₂; —S(═O)Y; —Z—S(═O)Y; —S(═O)₂Y; —Z—S(═O)₂Y; —S(═O)₃Y; —Z—S(3)₃Y; —P(═Z)(ZR)Y e.g. —P(═O)(OH)Y; —P(═Z)Y₂; —Z—P(═Z)(ZR)Y; —Z—P(═Z)Y₂; —P(═Z)(R)Y e.g. —P(═O)(H)Y; —Z—P(═Z)(R)Y; or —N═C(═Z) e.g. —N═C(═O).

Another group which may be formed naturally or synthetically on the biopolymer and which is bound to the biopolymer by one bond is —CN.

Other groups which may be formed naturally or synthetically on the biopolymer and which are bound to the biopolymer by one bond are: —P(ZR)Y e.g. —P(OH)Y; —PY₂; —Z—P(ZR)Y; —Z—PY₂; —P(R)Y e.g. —P(H)Y; —Z—P(R)Y. A particularly preferred group is —Z—P(ZR)Y, especially a phosphoramidite group:

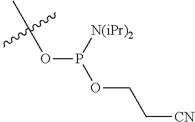

Another example of a group which may be formed naturally or synthetically on the biopolymer and which is bound to the biopolymer by one bond is —Y. In particular, when the reactive group is halo (especially iodo), the reactive group may be bound to an aliphatic or aromatic carbon.

Groups which may be formed synthetically on the biopolymer and which are bound to the biopolymer by two bonds include —N(R)— e.g. —NH—; —S—; —O—; —B(Y)—; —C(R)(Y)—; —CY₂—; —C(═O)—; —C(OH)(OR)—; —C(OR)₂—.

Groups which may be formed synthetically on the biopolymer and which are bound to the biopolymer by three bonds include

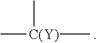

Preferred groups include nucleophilic groups, either natural or synthetic, e.g.: —NR₂ e.g. —NHR, especially —NH₂; —SR e.g. —SH; —OR e.g. —OH; —N(R)— e.g. —NH—; —S—; and —O—. The groups —NH₂, —SH and —OH are particularly preferred.

Another preferred reactive group is maleimidyl:

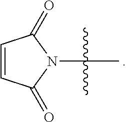

Y is independently a leaving group, including groups capable of leaving in an SN₂ substitution reaction or being eliminated in an addition-elimination reaction with the reactive group of the biopolymer B_{P}.

Preferred examples of Y include halogen (preferably iodo), C₁₋₈hydrocarbyloxy (e.g. C₁₋₈alkoxy), C₁₋₈hydrocarbyloxy substituted with one or more A, C₁₋₈heterohydrocarbyloxy, C₁₋₈heterohydrocarbyloxy substituted with one or more A, mesyl, tosyl, pentafluorophenyl, —O-succinimidyl (formula VII) or a sulfo sodium salt thereof (sulfoNHS—formula VIIIa), —S-succinimidyl, or phenyloxy substituted with one or more A e.g. p-nitrophenyloxy (formula VIII) or pentafluorophenoxy (formula VIIIa).

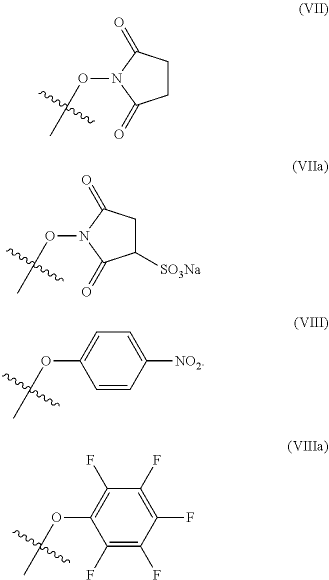

Thus, preferred reactive groups on the biopolymer are:

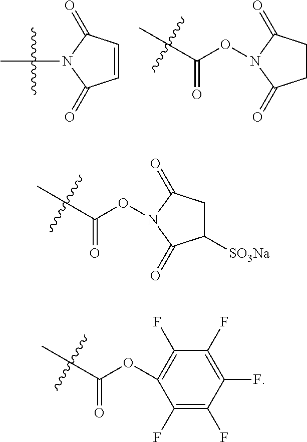

Other preferred examples of Y include —ZR. Particularly preferred examples of Y are —ZH (e.g. —OH or —NH₂) and —Z—C₁₋₈alkyl groups such as —NH—C₁₋₈alkyl groups (e.g. —NHMe) and —O—C₁₋₈alkyl groups (e.g. —O-t-butyl). Thus, preferred reactive groups are —C(O)—NH—C₁₋₈alkyl and —C(O)—O—C₁₋₈alkyl (e.g. —C(O)—O-t-butyl).

Other preferred examples of Y include —Z—ZR. Particularly preferred examples include —NR—NR₂, especially —NH—NH₂, and —ONR₂, especially —O—NH₂.

Z is independently O, S or N(R). Preferred (═Z) is (═O).

R is independently H, C₁₋₈hydrocarbyl (e.g. C₁₋₈alkyl) or C₁₋₈hydrocarbyl substituted with one or more A.

R is preferably H.

Other preferred reactive groups include —C(═O)Y, especially —C(═O)—O-succinimidyl and —C(═O)—O-(p-nitrophenyl).

In a further embodiment, the reactive group may be —Si(R)₂—Y, with Y being halo (e.g. chloro) being especially preferred. Preferred groups R in this embodiment are C₁₋₈alkyl, especially methyl. A particularly preferred reactive group in this embodiment is —Si(Me)₂Cl.

Other groups which may be formed naturally or synthetically on the biopolymer include groups capable of reacting in a cycloaddition reaction, especially a Diels-Alder reaction.

In the case of Diels-Alder reactions, the reactive group on the biopolymer is either a diene or a dienophile. Preferred diene groups are

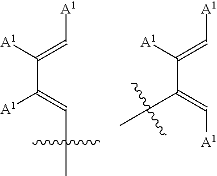

and multivalent derivatives formally formed by removal of one or more hydrogen atoms, where A¹ is —R¹ or —Z¹R¹, where R¹ and Z¹ are defined below.

Preferred dienophile groups are —CR¹═CR¹₂, —CR¹═C(R¹)A², —CA²═CR¹₂, —CA²═C(R¹)A² or —CA²=CA²₂, and multivalent derivatives formally formed by removal of one or more hydrogen atoms, where R¹ is defined below and A² is independently halogen, trihalomethyl, —NO₂, —CN, —N⁺(R¹)₂O—, —CO₂H, —CO₂R¹, —SO₃H, —SOR¹, —SO₂R¹, —SO₃R¹, —OC(═O)OR¹, —C(═O)H, —C(═O)R¹, —OC(═O)R¹, —OC(═O)NR¹₂, —N(R¹)C(═O)R¹, —C(═S)NR¹₂, —NR¹C(═S)R¹, —SO₂NR¹₂, —NR¹SO₂R¹, —N(R¹)C(═S)NR¹₂, or —N(R¹)SO₂NR¹₂, where R¹ is defined below. A particularly preferred dienophile group is maleimidyl.

### Group M

The group M is capable of reacting with the reactive group of the biopolymer, B_{P}, to form a covalent linkage. [Group ‘M’ is shown as ‘AFG’ in the drawings].

The group M is bound to L_{M} by one or more covalent bonds (e.g. 2 or 3 bonds, especially 2 such as

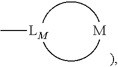

which are either single, double or triple covalent bonds (preferably single bonds).

Preferably, M is bound to L_{M} by one single bond.

Alternatively, or in addition, M is bound by more than one L_{M}, such L_{M} either being attached to the same or different Ar¹ or Ar². In a preferred embodiment M is bound by more than one L_{M} from different Ar¹ or Ar², e.g.:

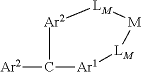

Examples of group M bound to L_{M} by one bond include —NR₂ e.g. —NBR, especially —NH₂; —SR e.g. —SH; —OR e.g. —OH; —B(R)Y; —BY₂; —C(R)₂Y; —C(R)Y₂; —CY₃; —C(═Z)Y e.g. —C(═O)Y; —Z—C(═Z)Y; —C(═Z)R e.g. —C(═Z)H, especially —C(═O)H; —C(R)(OH)OR; —C(R)(OR)₂; —S(═O)Y; —Z—S(═O)Y; —S(═O)₂Y; —Z—S(═O)₂Y; —S(═O)₃Y; —Z—S(O)₃Y; —P(═Z)(ZR)Y e.g. —P(═O)(OH)Y; —P(═Z)Y₂; —Z—P(═Z)(ZR)Y; —Z—P(═Z)Y₂; —P(═Z)(R)Y e.g. —P(═O)(H)Y; —Z—P(═Z)(R)Y; or —N═C(═Z) e.g. —N═C(═O).

Another example of a group M bound to L_{M} by one bond is —CN.

Other examples of group M bound to L_{M} by one bond are —P(ZR)Y e.g. —P(OH)Y; —PY₂; —Z—P(ZR)Y; —Z—PY₂; —P(R)Y e.g. —P(H)Y; —Z—P(R)Y. A particularly preferred group M is —Z—P(ZR)Y, especially a phosphoramidite group:

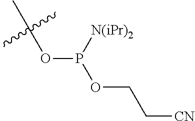

Another example of group M bound to L_{M} by one bond is Y. In particular, when group M is halo (especially iodo), M may be bound to an aliphatic or aromatic carbon. When M is halo (e.g. iodo) and is bound to an aromatic carbon, L_{M} may, for example, be a single bond.

Examples of group M bound to L_{M} by two bonds include —N(R)— e.g. —NH—; —S—; —O—; —B(Y)—; —C(R)(Y)—; —CY₂—; —C(═O)—; —C(OH)(OR)—; —C(OR)₂—.

Examples of group M bound to L_{M} by three bonds include

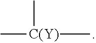

Preferred groups M include electrophilic groups, especially those susceptible to SN₂ substitution reactions, addition-elimination reactions and addition reactions, e.g. —B(R)Y; —BY₂; —C(R)₂Y; —C(R)Y₂; —CY₃; —C(═Z)Y e.g. —C(═O)Y; —Z—C(═Z)Y; —C(═Z)R e.g. —C(═Z)H, especially —C(═O)H; —C(R)(OH)OR; —C(R)(OR)₂; —S(═O)Y; —Z—S(═O)Y; —S(═O)₂Y; —Z—S(═O)₂Y; —S(═O)₃Y; —Z—S(═O)₃Y; —P(═Z)(ZR)Y e.g. —P(═O)(OH)Y; —P(═Z)Y₂; —Z—P(═Z)(ZR)Y; —Z—P(═Z)Y₂; —P(═Z)(R)Y e.g. —P(═O)(R)Y; —Z—P(═Z)(H)Y; —N═C(═Z) e.g. —N═C(═O); —B(Y)—; —C(R)(Y)—; —CY₂—; —C(═O)—; —C(OH)(OR)—; —C(OR)₂—; or

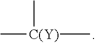

Another preferred electrophilic group M is —CN.

Still further preferred examples of group M are orthoesters, e.g. —C(OR)₃. In a preferred embodiment, the R groups are linked together to form a hydrocarbyl group, e.g. a C₁ alkyl group. A preferred example of group M in this embodiment is:

Another preferred group M is maleimido.

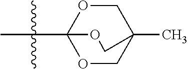

Y, Z and R are defined as above. Preferred Y groups when present on M are those capable of leaving in an SN₂ substitution reaction or being eliminated in an addition-elimination reaction with the reactive group of the biopolymer B_{P}.

Preferred examples of Y include halogen (preferably iodo), C₁₋₈hydrocarbyloxy (e.g. C₁₋₈alkoxy), C₁₋₈hydrocarbyloxy substituted with one or more A, C₁₋₈heterohydrocarbyloxy, C₁₋₈heterohydrocarbyloxy substituted with one or more A, mesyl, tosyl, pentafluorophenyl, —O-succinimidyl (formula VII) or a sulfo sodium salt thereof (sulfoNHS—formula VIIIa), —S-succinimidyl, or phenyloxy substituted with one or more A e.g. p-nitrophenyloxy (formula VIII) or pentafluorophenoxy (formula VIIIa).

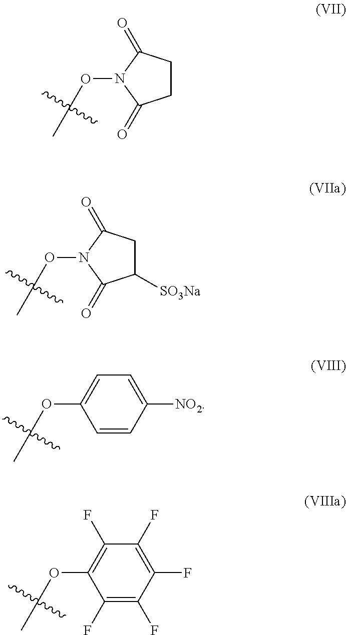

Thus, preferred groups M are:

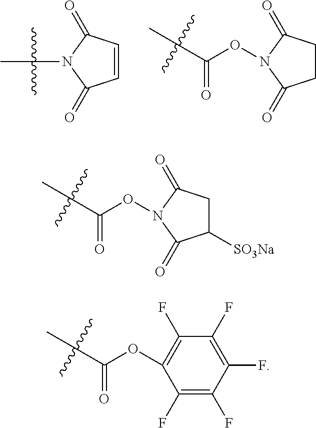

Other preferred examples of Y include —ZR. Particularly preferred examples of Y are —ZH (e.g. —OH or —NH₂) and —Z—C₁₋₈alkyl groups such as —NH—C₁₋₈alkyl groups (e.g. —NHMe) and —O—C₁₋₈alkyl groups (e.g. —O-t-butyl). Thus, preferred groups M are —C(O)—NH—C₁₋₈alkyl (e.g. —C(O)NHMe) and —C(O)—O—C₁₋₈alkyl (e.g. —C(O)—O-t-butyl).

Other preferred examples of Y include —Z—ZR. Particularly preferred examples include —NR—NR₂, especially —NH—NH₂, and —ONR₂, especially —O—NH₂.

Particularly preferred groups M include —C(═O)Y, especially —C(═O)—O-succinimidyl and —C(═O)—O-(p-nitrophenyl).

In a further embodiment, M may be —Si(R)₂—Y, with Y being halo (e.g. chloro) being especially preferred. Preferred groups R in this embodiment are C₁₋₈alkyl, especially methyl. A particularly preferred group M in this embodiment is —Si(Me)₂Cl.

In a further embodiment, M may be —C(Ar²)₂X. Preferred groups Ar² and X are set out below. In this embodiment it is preferred that L_{M} is a bond. A particularly preferred group M in this embodiment is:

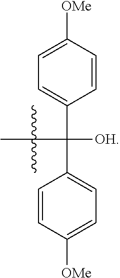

Other groups M include groups capable of reacting in a cycloaddition reaction, especially a Diels-Alder reaction.

In the case of Diels-Alder reactions, the reactive group on the biopolymer is either a diene or a dienophile. Preferred diene groups are

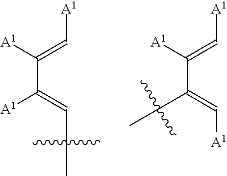

and multivalent derivatives formally formed by removal of one or more hydrogen atoms, where A¹ is —R¹ or —Z¹R¹, where R¹ and Z¹ are defined below.

Preferred dienophile groups are —CR¹═CR¹₂, —CR¹═C(R¹)A², —CA²═CR¹₂, —CA²═C(R¹)A² or —CA²═CA²₂, and multivalent derivatives formally formed by removal of one or more hydrogen atoms, where R¹ is defined below and A² is independently halogen, trihalomethyl, —NO₂, —CN, —N⁺(R¹)₂O⁻; —CO₂H, —CO₂R¹, —SO₃H, —SO₂R¹, —SO₃R¹, —OC(═O)OR¹, —C(═O)H, —C(═O)R¹, —OC(═O)R¹, —OC(═O)NR¹₂, —N(R¹)C(═O)R¹, —C(═S)NR¹₂, —NR¹C(═S)R¹, —SO₂NR¹₂, —NR¹SO₂R¹, —N(R¹)C(═S)NR¹₂, or —N(R¹)SO₂NR¹₂, where R¹ is defined below. A particularly preferred dienophile group is maleimidyl.

Preferred examples of group M are shown in FIGS. 11A and 11B.

### Matching B_{P} and M

The reactive group on the biopolymer [shown as ‘F’ in the drawings] and the group M [shown as ‘AFG’ in the drawings] must be dependently selected in order to form the covalent linkage. For example, where the biopolymer includes the groups —NH₂, —OH or —SH, M will typically be —B(R)Y; —BY₂; —C(R)₂Y; —C(R)Y₂; —CY₃; —C(═Z)Y e.g. —C(═O)Y; —Z—C(═Z)Y; —C(═Z)R e.g. —C(═Z)H, especially —C(═O)H; —C(R)(OH)OR; —C(R)(OR)₂; —S(═O)Y; —Z—S(═O)Y; —S(═O)₂Y; —Z—S(═O)₂Y; —S(═O)₃Y; —Z—S(═O)₃Y; —P(═Z)(ZR)Y e.g. —P(═O)(OH)Y; —P(═Z)Y₂; —Z—P(═Z)(ZR)Y; —Z—P(═Z)Y₂; —P(═Z)(R)Y e.g. —P(═O)(H)Y; —Z—P(═Z)(R)Y; —N═C(═Z) e.g. —N═C(═O); —B(Y)—; —C(R)(Y)—; —CY₂—; —C(═O)—; —C(OH)(OR)—; —C(OR)₂—; or

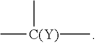

M may also be —CN.

In a preferred embodiment, one of the reactive group on the biopolymer and group M is a maleimidyl and the other will be a —SH group.

Alternatively, when the covalent linkage is to be formed by a Diels Alder reaction, one of the reactive group on the biopolymer and group M will typically be a diene and the other will be a dienophile.

Preferred covalent linkage are those produced through the reaction of the following groups:

| M | Group on Bp | Obtained Linkage M′—Bₚ′ |
|---|---|---|
| —C(═O)—O-succinimidyl | —NH₂ | —CO—NH— |
| [i.e. carboxy-NHS] | | |
| —C(═O)—O-(p-nitrophenyl) | —NH₂ | —CO—NH— |
| —C(═O)-pentafluorophenyl | —NH₂ | —CO—NH— |
| Biotin | avidin/streptavidin | biotin-(strept)avidin |
| 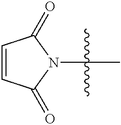 | —SH | 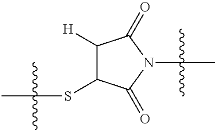 |
| —N═C═S (isothiocyanate) | —NH₂ | —NH—CS—NH— |

The covalent residue M′-B_{P}′ is the reaction product of M and B_{P}. B_{P}′ will generally be the same as B_{P} except that instead of the reactive group, B_{P}′ will have a residue of the reactive group covalently bound to the residue M′. Depending on the choice of the reactive group and the choice of M, M′ and the residue of the reactive group will typically form linkages, in the orientation L_{M}-M′-B_{P}′, including —C(R)₂Z—, —ZC(R)₂—, —C(═Z)Z—, —ZC(═Z)—, —ZC(═Z)Z—, —C(OH)(R)Z—, —ZC(OH)(R)—, —C(R)(OR)Z—, —ZC(R)(OR)—, —C(R)(OR)Z—, —ZC(R)(OR)—, —S(═O)Z—, —ZS(═O)—, —ZS(═O)Z—, —S(═O)₂Z—, —ZS(═O)₂—, —ZS(═O)₂Z—, —S(═O)₃Z—, —ZS(═O)₃—, —ZS(═O)₃Z—, —P(═Z)(ZR)Z—, —ZP(═Z)(ZR)—, —ZP(═Z)(ZR)Z—, —P(═Z)(R)Z—, —ZP(═Z)(R)—, —ZP(═Z)(R)Z—, —NH—C(═Z)—Z—, where Z and R are as defined above.

### Group M″

M″ is the same as M except that the group S_{S} is bound to a portion of M which does not form part of M′. Thus, M″ is a residue of M formable by the conjugation of M and S_{S}. However, M″ need not necessarily be formed by the conjugation of M and S_{S}.

M″—S_{S} comprises a covalent, ionic, dipole-dipole, hydrogen, or van der Waals bond. The covalent, ionic, hydrogen, dipole-dipole or van der Waals bond may be direct between M″ and S_{S} or may be provided by one or more binding groups present on M″ and/or S_{S}.

Examples of groups which can form these types of bond, and methods for cleaving these types of bond, are set out below in connection with C—S_{S} bonds, etc.

This embodiment of the invention is advantageous, since the derivativisation of the biopolymer will also release the derivatised biopolymer from the solid support. Thus, an additional step of cleaving the biopolymer from the solid support is not required.

Preferred groups M″ are groups M having a leaving group, wherein the group S_{S} is bound to the leaving group, e.g. groups M mentioned above having a leaving group Y, wherein the group S_{S} is bound to the leaving group Y.

A particularly preferred group M″ is:

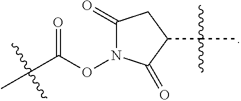

### L_{M}

Where the group L_{M} is a linker atom or group, it has a sufficient number of linking covalent bonds to link L_{M} to the group Ar¹ by a single covalent bond (or more, as appropriate) and to link L_{M} to the p instances of M (or M′, as appropriate) groups (which may be attached to L_{M} by one or more bonds).

The group L_{M} may be directly bound to the aromatic part of Ar¹, bound to one or more of the substituents A of Ar¹, or both. Preferably, L_{M} is bound directly to the aromatic part of Ar¹.

In an alternative embodiment, L_{M} may be bound to L₅.

When L_{M} is a linker atom, preferred linker atoms are O or S, particularly O.

When L_{M} is a linker group, preferred linker groups, in the orientation Ar¹-(L_{M}{M}ₚ)_{q} or Ar¹-(L_{M}{M′}ₚ)_{q}, as appropriate, are -E^{M}-, -(D^{M})ₜ-, -(E^{M}-D^{M})ₜ-, -(D^{M}-E^{M})ₜ-, -E^{M}-(D^{M}-E^{M})ₜ- or -D^{M}-(E^{M}-D^{M})ₜ-, where a sufficient number of linking covalent bonds, in addition to the covalent bonds at the chain termini shown, are provided on groups E^{M} and D^{M} for linking the p instances of M (or M′) groups.

D^{M} is independently C₁₋₈hydrocarbylene or C₁₋₈hydrocarbylene substituted with one or more A. Preferred D^{M} are C₁₋₈alkylene, C₁₋₈alkenylene and C₁₋₈alkynylene, especially C₁₋₈alkylene and C₁₋₈ alkynylene, each optionally substituted with one or more A (preferably unsubstituted). A preferred substituent A is ²H. Preferred L_{M} in the orientation Ar¹-(L_{M}{M}ₚ)_{q} or Ar¹-(L_{M}{M′}ₚ)_{q}, as appropriate, are: —CH₂CH₂—; —C≡C—CH₂CH₂CH₂—; —(CH₂)₅—; —CD₂CD₂CH₂CH₂CH₂—; —C≡C—CH₂— and —CH₂CH₂CH₂—.

E^{M}, in the orientation Ar¹-(L_{M}{M}ₚ)_{q} or Ar¹-(L_{M}{M′})ₚ)_{q}, as appropriate, is independently —Z^{M}—, C(═Z^{M})—, —Z^{M}C(═Z^{M})—, —C(═Z^{M})Z^{M}—, —Z^{M}C(═Z^{M})Z^{M}—, —S(═O)—, —Z^{M}S(═O)—, —S(═O)Z^{M}—, —Z^{M}S(═O)Z^{M}—, —S(═O)₂—, —Z^{M}S(═O)₂—, —S(═O)₂Z^{M}—, —Z^{M}S(═O)₂Z^{M}—, where Z^{M} is independently O, S or N(R^{M}) and where R^{M} is independently H, C₁₋₈hydrocarbyl (e.g. C₁₋₈alkyl) or C₁₋₈hydrocarbyl substituted with one or more A. Preferably E^{M} is, in the orientation Ar¹-(L_{M}{M}ₚ)_{q}, or Ar¹-(L_{M}{M′}ₚ)_{q}, as appropriate, —O—, —S—, —C(═O)O—, —C(═S)—, —C(═S)O—, —OC(═S)—, —C(═O)S—, —S(O)—, —S(O)₂—, —NR^{M}—, —C(═O)N(R^{M})—, —C(═S)N(R^{M})—, —N(R^{M})C(═O)—, —N(R^{M})C(═S)—, —S(═O)N(R^{M})—, —N(R^{M})S(═O)—, —S(═O)₂N(R^{M})—, —N(R^{M})S(═O)₂—, —OC(═O)O—, —SC(═O)O—, —OC(═O)S—, —N(R^{M})C(═O)O—, —OC(═O)N(R^{M})—, —N(R^{M})C(═O)N(R^{M})—, —N(R^{M})C(═S)N(R^{M})—, —N(R^{M})S(═O)N(R^{M})— or —N(R^{M})S(═O)₂N(R^{M})—.

Alternative groups E^{M} to those defined above, in the orientation Ar¹-(L_{M}{M}ₚ)_{q} or Ar¹-(L_{M}{M′}ₚ)_{q}, as appropriate, are —Z^{M}—Si(R^{M})₂—Z^{M}—, —Si(R^{M})₂—Z^{M}- and —Z^{M}—Si(R^{M})₂—. The group —Si(R^{M})₂—Z^{M}— is particularly preferred. Z^{M} is preferably O. R^{M} is preferably C₁₋₈alkyl, preferably methyl. These groups E^{M} are particularly preferred in the groups -(E^{M}-D^{M})ₜ-, especially when t=1 and D^{M} is C₁₋₈alkylene. The following group is especially preferred:

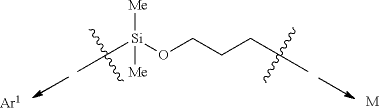

In addition to the above definition of D^{M}, D^{M} may also be C₁₋₈heterohydrocarbylene or C₁₋₈heterohydrocarbylene substituted with one or more A. In this embodiment, C₁₋₈cycloheteroalkylene groups are particularly preferred, e.g.:

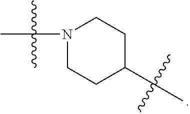

Thus, preferred L_{M} groups -D^{M}-E^{M}-D^{M}- are, in the orientation Ar¹-(L_{M}{M}ₚ)_{q} or Ar¹-(L_{M}{M′}ₚ)_{q}, as appropriate, —C₁₋₈alkylene-C(O)—C₁₋₈cycloheteroalkylene (preferably where the hetero atom is N and is bound to the carboxy), especially:

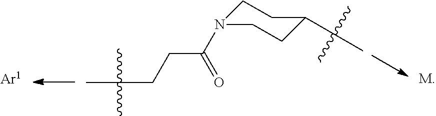

t=1 or more, e.g. from 1 to 50, 1 to 40, 1 to 30, 1 to 20 or 1 to 10. Preferably t=1, 2, 3, 4, 5, 6, 7, 8, 9, or 10.

Preferably, L_{M} links one group M (or Mi) to Ar¹, M (or M′) is linked to L_{M} by a single covalent bond and therefore no additional bonds are required (e.g. L_{M}{M}₁ may be -E^{M}-{M}), -(D^{M})ₜ-{M}, -(E^{M}-D^{M})ₜ-{M}, -(D^{M}-E^{M})ₜ-{M}, -E^{M}-(D^{M}-E^{M})ₜ-{M} or -D^{M}-(E^{M}-D^{M})ₜ-{M}).

Where L_{M} includes a group which also falls within the definition of group M, the group M is preferably more reactive than the group included in L_{M}.

L_{M} is preferably -(D^{M})ₜ-, -(E^{M}-D^{M})ₜ-, or -D^{M}-(E^{M}-D^{M})ₜ-.

When group L_{M} is -(D^{M})ₜ-, t is preferably 1. D^{M} is preferably C₁₋₈alkylene, preferably methylene or ethylene.

When group L_{M} is -(E^{M}-D^{M})ₜ-, or -D^{M}-(E^{M}-D^{M})ₜ-, E^{M} is preferably (in the orientation Ar¹-(L_{M}{M}ₚ)_{q} or Ar¹-(L_{M}{Mi}ₚ)_{q}, as appropriate), —C(═O)N(R^{M})— (e.g. —C(═O)NH—) or O (preferably O), and D^{M} is preferably C₁₋₈alkylene, preferably ethylene, propylene, butylene or pentylene (preferably ethylene or propylene). t is preferably 1. Especially preferred L_{M} are, in the orientation Ar¹-(L_{M}{M}ₚ)_{q} or Ar¹-(L_{M}{M′}ₚ)_{q}, as appropriate, —O—CH₂CH₂CH₂— and —O—CH₂CH₂CH₂CH₂CH₂—.

Another preferred group -D^{M}-(E^{M}-D^{M})ₜ- is where D^{M} is C₁₋₈alkylene and t is 1. Preferred E^{M} in this group, in the orientation Ar¹-(L_{M}{M}ₚ)_{q} or Ar¹-(L_{M}{M′}ₚ)_{q}, as appropriate, are —Z^{M}C(═Z^{M})— (especially —N(R^{M})C(═O)—, e.g. —N(Me)C(═O)—) and —C(═Z^{M})Z^{M}-(especially —C(═O)O—). Particularly preferred L_{M} groups are:

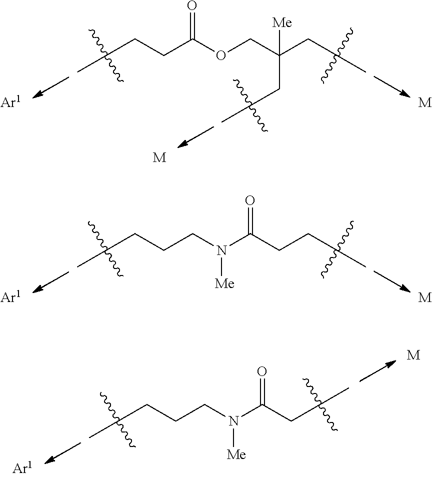

The group -(E^{M}-D^{M})ₜ- is preferred, a particularly preferred example of which is (in the orientation Ar¹-(L_{M}{M}ₚ)_{q} or Ar₁-L_{M}{M′}ₚ)_{q}, as appropriate) —C(═O)NH—CH₂CH₂CH₂—O—CH₂CH₂—O—CH₂CH₂—O—CH₂CH₂CH₂—.

In an alternative embodiment it is preferred that L_{M} is a single covalent bond.

When Ar² is phenyl, L_{M} is preferably provided in a position ortho or para to C★. When Ar² is other than phenyl, L_{M} is preferably attached to an atom which bears the charge in at least one of the resonance structures of the ions of formula (I).

Where C★ is a cation, L_{M} is preferably an electron-donating group. Where C★ is an anion, L_{M} is preferably an electron-withdrawing group.

Preferred examples of L_{M} are shown in FIGS. 10A and 10B.

### C—S_{S}, S_{S}—Ar¹ and S_{S}—Ar² Bonds

C—S_{S}, S_{S}—Ar¹ and S_{S}—Ar² comprise a cleavable covalent, ionic, hydrogen, dipole-dipole or van der Waals bond (also known as a dispersion bond or a London forces bond). The covalent, ionic, hydrogen, dipole-dipole or van der Waals bond may be direct between C and S_{S}, Ar¹ and S_{S}, or Ar² and S_{S}, or may be provided by one or more binding groups present on C and/or S_{S}, Ar¹ and/or S_{S}, or Ar² and/or S_{S}, respectively.

In addition, however, the C—S_{S} bond comprises a cleavable bond between C and S_{S} involving a thioether sulphur atom bound directly to the α-carbon atom, i.e. without any other intervening atoms between the thioether sulphur atom and the α-carbon atom, and the definitions of the bonds below are applicable to the definition of the C—S_{S} bond provided they involve a thioether sulphur atom bound directly to the α-carbon atom.

### Covalent Bonding

Where the bond is covalent, the bond may be direct (e.g. C—S_{S}, Ar¹—S_{S} or Ar²—S_{S}, respectively) or may be provided by a linker atom or group L⁴ (e.g. C-L⁴-S_{S}, Ar¹-L⁴-S_{S} or Ar²-L⁴-S_{S}, respectively).

When L⁴ is a linker group, preferred linker groups are -E⁴-, (D₄)_{t″}-, -E⁴-D₄)_{t″}, -D⁴-E₄)_{t″}, -E⁴-D⁴-E₄)_{t″} or -D⁴-E⁴-D₄)_{t″}.

D⁴ is independently C₁₋₈hydrocarbylene or C₁₋₈hydrocarbylene substituted with one or more A.

E⁴ is, in the orientation C-L⁴-S_{S}, independently —Z⁴—, —Z⁴C(═Z⁴)—, —C(═Z⁴)Z⁴—, —Z⁴C(═Z⁴)Z⁴—, —S(═O)—, —Z⁴S(═O)—, —S(═O)Z⁴—, —Z⁴S(═O)Z⁴—, —S(═O)₂—, —Z⁴S(═O)₂—, —S(═O)₂Z⁴—, —Z⁴S(═O)₂Z⁴—, where Z⁴ is independently O, S or N(R⁴), and where R⁴ is independently H, C₁₋₈hydrocarbyl (e.g. C₁₋₈alkyl) or C₁₋₈hydrocarbyl substituted with one or more A. Preferably E⁴ is, in the orientation C-L⁴-S_{S}, —O—, —S—, —C(═O)—, —C(═O)O—, —C(═S)—, —C(═S)O—, —OC(═S)—, —C(═O)S—, —SC(═O)—, —S(O)—, —S(O)₂—, —N(R⁴)—, —C(═O)N(R⁴)—, —C(═S)N(R⁴)—, —N(R⁴)C(═O)—, —N(R⁴)C(═S)—, —S(═O)N(R⁴)—, —N(R⁴)S(═O)—, —S(═O)₂N(R⁴)—, —N(R⁴)S(═O)₂—, —OC(═O)O—, —SC(═O)O—, —OC(═O)S—, —N(R⁴)C(═O)O—, —OC(═O)N(R⁴)—, —N(R⁴)C(═O)N(R⁴)—, —N(R⁴)C(═S)N(R⁴)—, —N(R⁴)S(═O)N(R⁴)— or —N(R⁴)S(═O)₂N(R⁴)—.

t″=1 or more, e.g. from 1 to 50, 1 to 40, 1 to 30, 1 to 20 or 1 to 10. Preferably t″=1, 2, 3, 4, 5, 6, 7, 8, 9, or 10.

Where L⁴ includes a group which also falls within the definition of group M, the group M is preferably more reactive than the group included in L⁵.

L⁴ is preferably a linker atom, preferably O or S, particularly O.

When the solid support S_{S} is gold, L⁴ is preferably covalently attached to the S_{S} by a sulphide or disulphide group.

When the cleavable bond of C—S_{S} bond is covalent and direct, the S_{S} solid support comprises a thioether sulphur atom bound directly to α-carbon atom. When the bond is provided by a linker atom or group L⁴, L⁴ comprises a thioether sulphur atom bound directly to the α-carbon atom, e.g. L⁴ is -E⁴-, -(E⁴-D⁴)_{t″}, or -E⁴-(D⁴-E⁴)_{t″}, as defined above but wherein the left hand -E⁴- is —S—. A preferred L⁴ is this embodiment is —S—. Other preferred L⁴ include a group comprising a thioether sulphur atom linked to the α-carbon atom and a secondary alcohol, e.g. a group of the formula:

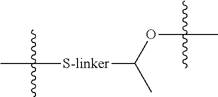

wherein the linker is a hydrocarbylene group or a heterohydrocarbylene group. Preferred hydrocarbylene groups include alkylenearylene groups (e.g.

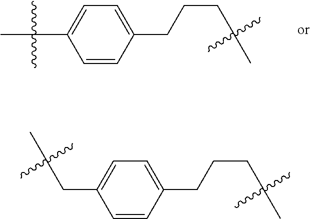

wherein the left hand side is attached to the sulphur atom) or alkylene groups (e.g. propylene).

### Ionic Bonding

Where the bond is ionic, the bond is typically direct (e.g. C★S_{S}★, where S_{S}★ is a solid support counterion to C★).

Alternatively, it may be provided by binding groups, e.g. chelating ligands, present on C or S_{S}, Ar¹ or S_{S}, or Ar² or S_{S}, respectively. In the case of C—S_{S} bonds, the chelating ligand is typically only present on S_{S} and chelates with C★.

Suitable chelating ligands which can bind anions include polyamines and cryptands.

Suitable chelating ligands which can bind cations include polyacidic compounds (e.g. EDTA) and crown ethers.

### Hydrogen Bonding

Where the bond is a hydrogen bond, the bond is usually provided by binding groups present on C or S_{S}, Ar¹ or S_{S}, or Ar² or S_{S}, respectively.

Typically, in order to form the hydrogen bond, one of C or S_{S}, Ar¹ or S_{S}, or Ar² or S_{S}, as appropriate, will have a binding group bearing one or more hydroxy, amino or thio hydrogen atoms, and the other of C or S_{S}, Ar¹ or S_{S}, or Ar² or S_{S}, respectively, will have a binding group bearing an atom having one or more lone pair of electrons (e.g. an oxygen, sulphur or nitrogen atom). Preferably, one of C or S_{S}, Ar¹ or S_{S}, or Ar² or S_{S}, as appropriate, will have a binding group comprising biotin, and the other of C or S_{S}, Ar¹ or S_{S}, or Ar² or S_{S}, respectively, will have a binding group comprising avidin or streptavidin.

Alternatively, the hydrogen bond may be direct.

### Dipole-Dipole Bonding

Where the bond is a dipole-dipole bond, it may be formed between permanent dipoles or between a permanent dipole and an induced dipole.

Typically, in order to form the dipole-dipole bond, one of S_{S} and the compound of the invention has a permanent dipole and the other of S_{S} and the compound of the invention has an induced dipole or a permanent dipole, the attraction between the dipoles forming a dipole-dipole bond.

Preferably, S_{S} comprises binding groups (e.g acid groups, —(NMe₃)⁺, carboxy, carboxylate, phosphate or sulphate groups) which produce a dipole at the surface of the solid support to bind the compound of the invention.

### Van der Waals Bonding

Where the bond is a van der Waals bond, the bonding is usually provided by binding groups present on C or S_{S}, Ar¹ or S_{S}, or Ar² or S_{S}, respectively.

Typically, in order to form the van der Waals bond, at least one, but preferably both, of C or S_{S}, Ar¹ or S_{S}, or Ar² or S_{S}, as appropriate, will have a hydrocarbyl or heterohydrocarbyl group (usually a large hydrocarbyl group having at least ten carbon atoms up to about 50 carbon atoms), optionally substituted with one or more A. Polyfluorinated hydrocarbyl and heterohydrocarbyl groups are particularly preferred. Typically, the hydrocarbyl or heterohydrocarbyl groups are aryl or heteroaryl groups or groups of the formula —C(R⁶)₂Ar³, —C(R⁶)(Ar³)₂ or —C(Ar³)₃, where Ar³ is independently defined the same as Ar² and R⁶ is H, C₁₋₈ hydrocarbyl, C₁₋₈ hydrocarbyl substituted by one or more A, C₁₋₈ heterohydrocarbyl or C₁₋₈ heterohydrocarbyl substituted by one or more A.

A preferred binding group is tetrabenzofullerene (formula X).

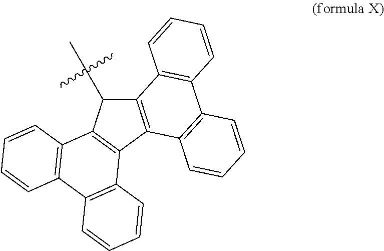

Alternatively, the van der Waals bond may be direct.

### Bond Cleavage

Preferably, the ions of formula (I) have a pKᵣ₊ value of at least zz, where zz is 0 or more (e.g. 0, 1, 2, 3, 4, 5, 6, 7, 8, 9, 10, 11, 12, 13, 14). More preferably, zz is 1 or more, still more preferably 2 or more, still more preferably 3 or more.

Preferably, the compounds of formula (IIa) or (IIIa) or the solid supports of formula (IVai), (IVaii), (IVaiii) or (IVaiv) provide ions of formula (I) having a pKᵣ₊ value of at least zz, where zz is defined above.

### C—X Bonds

The C—X bonds are cleavable by irradiation, electron bombardment, electrospray, fast atom bombardment (FAB), inductively coupled plasma (ICP) or chemical ionisation. Preferably, the C—X bonds are cleavable by irradiation or chemical ionisation.

The term ‘irradiation’ includes, for example, laser illumination, in particular as used in MALDI mass spectrometry. Laser light of about 340 nm is particularly preferred because it is typically used in MALDI mass spectrometers.

The term ‘electron bombardment’ includes, for example, bombardment with electrons having energy of about 70 ev.

Although the compounds of the invention are relatively stable to acid to allow attachment of the biopolymer without ionisation of the compound, chemical ionisation can nevertheless be effected, for example, by treatment with more acidic and/or more selective acid or acidic matrices (e.g. acidic matrices used in MALDI analysis).

Preferably, the step of cleaving the C—X bond or C—S_{S} bond in the methods of the invention (and preferably all steps) are carried out in the absence of an acidic matrix (e.g. an acidic matrix used in MALDI analysis), and preferably in the absence of any matrix.

Group X comprises a thioether sulphur atom bound directly to the α-carbon and is capable of being cleaved from the α-carbon atom to form an ion of formula (I).

Preferably, group X is sulfanyl, hydrocarbylsufanyl (e.g. C₁₋₁₄hydrocarbylsufanyl, especially C₁₋₈hydrocarbylsufanyl), hydrocarbylsufanyl (e.g. C₁₋₁₄hydrocarbylsufanyl, especially C₁₋₈hydrocarbylsufanyl) substituted with one or more A, heterohydrocarbylsufanyl (e.g. C₁₋₁₄heterohydrocarbylsufanyl, especially C₁₋₈heterohydrocarbylsufanyl) or heterohydrocarbylsufanyl (e.g. C₁₋₁₄heterohydrocarbylsufanyl, especially C₁₋₈heterohydrocarbylsufanyl) substituted with one or more A.

Preferred hydrocarbylsulfanyl groups include: alkylsulfanyl groups, e.g. alkylsulfanyl groups substituted by a hydroxyl group [especially a secondary hydroxyl group, e.g. —S(CH₂)₃CH(OH)CH₃)]; arylalkylsulfanyl groups, e.g. phenylalkylsulfanyl groups [especially benzylsulfanyl and phenylethylsulfanyl]; and arylsulfanyl groups, e.g. phenylsulfanyl. More preferred hydrocarbylsulfanyl groups are alkylsulfanyl groups and arylalkylsulfanyl groups, especially arylalkylsulfanyl groups.

Another preferred group X is an amidite linker comprising a thioether sulphur atom linked to the α-carbon atom and a phosphorylated secondary alcohol, e.g. of the formula:

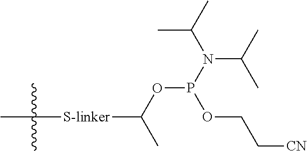

wherein the linker is a hydrocarbylene group or a heterohydrocarbylene group. Preferred hydrocarbylene groups include alkylenearylene groups (e.g.

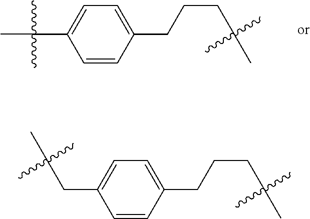

wherein the left hand side is attached to the sulphur atom) or alkylene groups (e.g. propylene).

In another embodiment X may be —S-succinimidyl.

### C—S_{S}, S_{S}—Ar¹ or S_{S}—Ar²

The C—S_{S}, S_{S}—Ar¹ or S_{S}—Ar² bonds are cleavable by irradiation, electron bombardment, electrospray, fast atom bombardment (FAB), inductively coupled plasma (ICP) or chemical ionisation. Preferably, the C—S_{S}, S_{S}—Ar¹ or S_{S}—Ar² bonds are cleavable by irradiation or chemical ionisation.

Where appropriate, the C—S_{S}, S_{S}—Ar¹ or S_{S}—Ar² bonds may be cleaved simultaneously or sequentially with the cleaving of the C—X bond by selection of suitable cleaving/dissociating conditions.

In one embodiment of the invention, the C—S_{S} bond in the solid support of formula (Vai) may be cleaved in sub-steps of step (iia) so that in a first sub-step a residue X (where X is the leaving group defined above) is provided and in a second subsequent sub-step the C—X bond is cleaved thereby forming the ion of formula (I). If desired, the second sub-step may be carried out substantially (e.g. seconds, minutes, hours or even days) after the first sub-step.

### Ar¹ and Ar²

### Ar²

Ar² is independently an aromatic group or an aromatic group substituted with one or more A and is preferably independently cyclopropyl, cyclopropyl substituted with one or more A, aryl, aryl substituted with one or more A, heteroaryl, or heteroaryl substituted with one or more A.

Where aryl or substituted aryl, Ar² is preferably C₆₋₃₀ aryl or substituted C₆₋₃₀ aryl. Where heteroaryl or substituted heteroaryl, Ar² is preferably C₆₋₃₀ heteroaryl or substituted C₆₋₃₀ heteroaryl.

Examples of aryl and heteroaryl are monocyclic aromatic groups (e.g. phenyl or pyridyl), fused polycyclic aromatic groups (e.g. napthyl, such as 1-napthyl or 2-napthyl) and unfused polycyclic aromatic groups (e.g. monocyclic or fused polycyclic aromatic groups linked by a single bond, a double bond, or by a —(CH═CH)ᵣ— linking group, where r is one or more (e.g. 1, 2, 3, 4 or 5).

Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene, which groups may be optionally substituted by one or more A.

Other examples of heteroaryl groups are monovalent derivatives of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene, which groups may be optionally substituted by one or more A. Preferred heteroaryl groups are five- and six-Membered monovalent derivatives, such as the monovalent derivatives of furan, imidazole, isothiazole, isoxazole, pyran, pyrazine, pyrazole, pyridazine, pyridine, pyrimidine, pyrrole, pyrrolizine and thiophene. The five-membered monovalent derivatives are particularly preferred, i.e. the monovalent derivatives of furan, imidazole, isothiazole, isoxazole, pyrazole, pyrrole and thiophene. The heteroaryl groups may be attached to the remainder of the compound by any carbon or hetero (e.g. nitrogen) atom.

Ar² is preferably C₆₋₃₀aryl substituted by one or more A, preferably phenyl or napthyl (e.g. 1-napthyl or 2-napthyl, especially 2-napthyl) substituted by one or more A, more preferably phenyl substituted by one or more A. When Ar² is phenyl, A is preferably provided in a position ortho or para to C★. When Ar² is other than phenyl, A is preferably attached to an atom which bears the charge in at least one of the resonance structures of the ions of formula (I).

Fused polycyclic aromatic groups, optionally substituted with one or more A, are particularly preferred.

A particularly preferred Ar² is unsubstituted pyrenyl or pyrenyl substituted with one or more A. Unsubstituted pyrenyl is preferred. The pyrenyl group may be 1-pyrenyl, 2-pyrenyl or 4-pyrenyl.

Preferred heteroaryl Ar² groups, whether substituted or unsubstituted, are pyridyl, pyrrolyl, thienyl and furyl, especially thienyl.

A preferred Ar² group is thiophenyl or thiophenyl substituted with one or more A. Unsubstituted thiophenyl is preferred. Examples of thiophenyl are thiophen-2-yl and thiophen-3-yl, with thiophen-2-yl being especially preferred.

When substituted, Ar² is preferably substituted by 1, 2 or 3 A. Ar² is preferably:

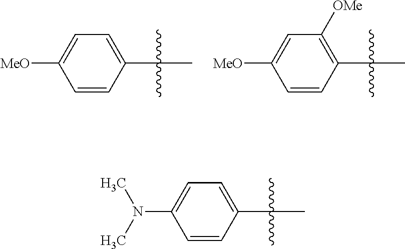

When unsubstituted, Ar² is preferably:

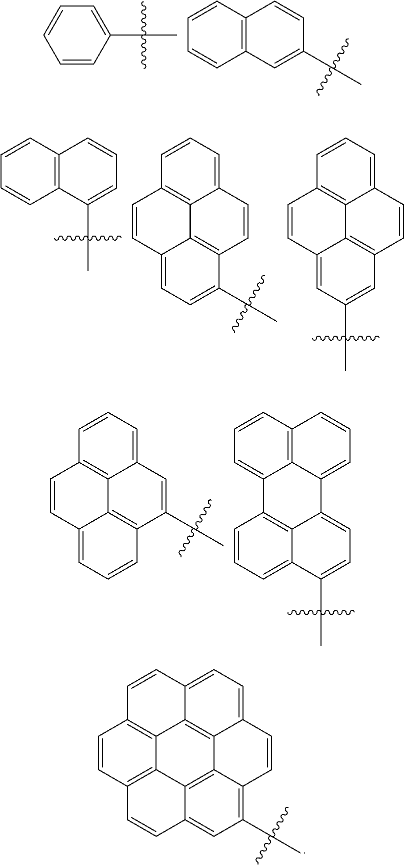

In another preferred embodiment, Ar² is cyclopropyl or cyclopropyl substituted with one or more A. Unsubstituted cyclopropyl is preferred. One or more, preferably one, of Ar² may be cyclopropyl.

Preferred examples of grotip Ar² are shown in FIGS. 12A and 12B.

### Ar₁

Ar¹ is independently an aromatic group or an aromatic group substituted with one or more A. The definition of Ar¹ is the same as Ar² (as defined above), except that the valency of the group Ar¹ is adapted to accommodate the q instances of the linker L_{M}. Preferred Ar² groups are also preferred Ar¹ groups, (as defined above), except that the valency of the group Ar¹ is adapted to accommodate the q instances of the linker L_{M}.

When q=1, Ar¹ is a divalent radical and is preferably independently cyclopropylene, cyclopropylene substituted with one or more A, arylene, arylene substituted with one or more A, heteroarylene, or heteroarylene substituted with one or more A.

Where arylene or substituted arylene, Ar¹ is preferably C₆₋₃₀ arylene or substituted C₆₋₃₀ arylene. Where heteroarylene or substituted heteroarylene, Ar¹ is preferably C₆₋₃₀ heteroarylene or substituted C₆₋₃₀ heteroarylene.

Examples of arylene and heteroarylene are monocyclic aromatic groups (e.g. phenylene or pyridylene), fused polycyclic aromatic groups (e.g. napthylene) and unfused polycyclic aromatic groups (e.g. monocyclic or fused polycyclic aromatic groups linked by a single bond, a double bond, or by a —(CH═CH)ᵣ— linking group, where r is one or more (e.g. 1, 2, 3, 4 or 5).

Other examples of arylene groups are polyvalent derivatives (where the valency is adapted to accommodate the q instances of the linker L_{M}) of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene, which groups may be optionally substituted by one or more A.

Other examples of heteroarylene groups are polyvalent derivatives (where the valency is adapted to accommodate the q instances of the linker L_{M}) of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene, which groups may be optionally substituted by one or more A. Preferred heteroaryl groups are five- and six-membered polyvalent derivatives, such as the polyvalent derivatives of furan, imidazole, isothiazole, isoxazole, pyran, pyrazine, pyrazole, pyridazine, pyridine, pyrimidine, pyrrole, pyrrolizine and thiophene. The five-membered polyvalent derivatives are particularly preferred, i.e. the polyvalent derivatives of furan, imidazole, isothiazole, isoxazole, pyrazole, pyrrole and thiophene. The heteroaryl groups may be attached to the remainder of the compound by any carbon or hetero (e.g. nitrogen) atom.

Ar¹ is preferably C₆₋₃₀arylene substituted by one or more A, preferably phenylene or napthylene substituted by one or more A, more preferably phenylene substituted by one or more A. When Ar¹ is phenylene, A is preferably provided in a position ortho or para to C★. When Ar¹ is other than phenylene, A is preferably attached to an atom which bears the charge in at least one of the resonance structures of the ions of formula (I).

When substituted, Ar¹ is preferably substituted by 1, 2 or 3 A.

When unsubstituted, preferred Ar¹ are:

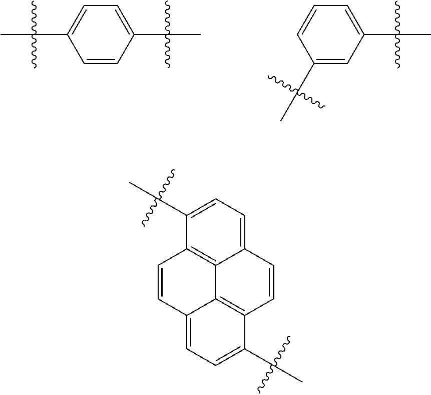

Preferred examples of group Ar¹ are shown in FIGS. 12A and 12B.

### Combinations of Ar

Optionally two or three of the groups Ar¹ and Ar² are linked together by one or more L⁵, where L⁵ is independently a single bond or a linker atom or group; and/or two or three of the groups Ar¹ and Ar² together form an aromatic group or an aromatic group substituted with one or more A.

When L⁵ is a linker group, preferred linker groups are -E⁵-, -(D⁵)_{t′}—, -(E⁵-D⁵)_{t′}-, -(D⁵-E⁵)_{t′}-, -E⁵-(D⁵-E⁵)_{t′}- or -D⁵-(E⁵-D⁵)ₜ′-.

D⁵ is independently C₁₋₈hydrocarbylene or C₁₋₈hydrocarbylene substituted with one or more A.

E⁵ is independently —Z⁵—, —C(═Z⁵)—, —Z⁵C(═Z⁵)—, —C(═Z⁵)Z⁵—, —Z⁵C(═Z⁵)Z⁵—, —Z⁵S(═O)—, —S(═O)Z⁵—, —Z⁵S(═O)Z⁵—, —Z⁵S(═O)₂—, —S(D)₂Z⁵—, —Z⁵S(═O)₂Z⁵—, where Z⁵ is independently O, S or N(R⁵) and where R⁵ is independently H, C₁₋₈hydrocarbyl or C₁₋₈hydrocarbyl substituted with one or more A. Preferably E⁵ is —O—, —S—, —C(═O)O—, —C(═S)O—, —OC(═S)—, —C(═O)S—, —SC(═O)—, —S(O)—, —S(O)₂—, —N(R⁵)—, —C(═O)N(R⁵)—, —C(═S)N(R⁵)—, —N(R⁵)C(═O)—, —N(R⁵)C(═S)—, —S(═O)N(R⁵)—, —N(R⁵)S(═O)—, —S(═O)₂N(R⁵)—, —N(R⁵)S(═O)₂—, —OC(═O)O—, —SC(═O)O—, —OC(═O)S—, —N(R⁵)C(═O)O—, —OC(═O)N(R⁵)—, —N(R⁵)C(═O)N(R⁵)—, —N(R⁵)C(═S)N(R⁵)—, —N(R⁵)S(═O)N(R⁵)— or —N(R⁵)S(═O)₂N(R⁵)—.

t′=1 or more, e.g. from 1 to 50, 1 to 40, 1 to 30, 1 to 20 or 1 to 10. Preferably t′=1, 2, 3, 4, 5, 6, 7, 8, 9, or 10. Most preferably f=1.

Where L⁵ includes an atom or group which also falls within the definition of group M, the group M is preferably more reactive than the group included in L⁵.

L⁵ is preferably a linker atom, preferably O or S, particularly O.

When L⁵ is a linker group, a preferred L⁵ is —N(R⁵)—.

In another embodiment in which L⁵ is a linker group, L⁵ is —S(═O)—. When two of the groups Ar¹ and Ar² are linked together by one or more (e.g. 2, 3 or 4) L⁵, they are preferably linked together by one L⁵, preferably O.

Preferred combinations of Ar are two Ar² (e.g. two Are phenyl groups) linked together by one L⁵ (e.g. O or S).

Particularly preferred combinations of Ar are two Ar² phenyl groups, optionally substituted by one or more A (preferably unsubstituted), linked together by one L⁵ (e.g. O or S), where is L⁵ is ortho to C★ with respect to both phenyl groups. Especially preferred combinations of two Ari groups are:

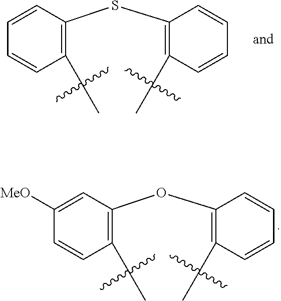

In another embodiment, at least one L_{M} is linked to an atom or group L⁵. In this embodiment, the preferred L⁵ mentioned above are, where appropriate, modified to remove substituents R⁵ in order to accommodate L_{M}, e.g. the R⁵ substituent of the group —N(R⁵)— is replaced by L_{M}. In this embodiment, the L⁵ group to which L_{M} is bound is preferably:

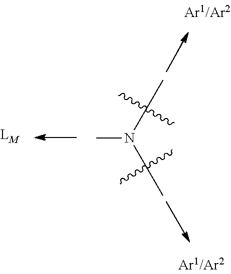

Preferred combinations of Ar¹ and/or Ar² in this embodiment are:

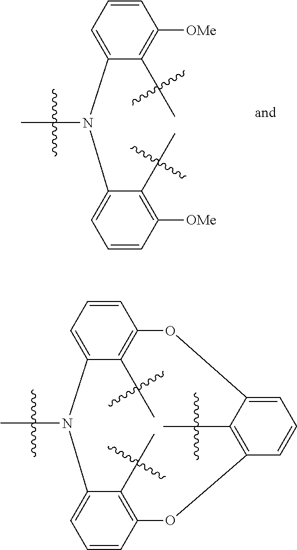

When two or three of the groups Ar¹ and Ar² together form an aromatic group or an aromatic group substituted with one or more A, the aromatic group may be a carbocyclic aromatic group or a carbocyclic aromatic group in which one or more carbon atoms are each replaced by a hetero atom. Typically, in an aromatic group in which one or more carbon atoms are each replaced by a hetero atom, up to three carbons are so replaced, preferably up to two carbon atoms, more preferably one carbon atom.

Preferred hetero atoms are O, Se, S or N, more preferably O, S or N.

When two or three of the groups Ar¹ and Ar₂ together form an aromatic group or an aromatic group substituted with one or more A, preferred aromatic groups are C₈₋₅₀ aromatic groups.

The aromatic groups may be monocyclic aromatic groups (e.g. radicals of suitable valency derived from benzene), fused polycyclic aromatic groups (e.g. radicals of suitable valency derived from napthalene) and unfused polycyclic aromatic groups (e.g. monocyclic or fused polycyclic aromatic groups linked by a single bond, a double bond, or by a —(CH═CH)ᵣ linking group, where r is one or more (e.g. 1, 2, 3, 4 or 5).

When two or three of the groups Ar¹ and Ar² together form a carbopolycyclic fused ring aromatic group, preferred groups are radicals of suitable valency obtained from napthalene, anthracene or phenanthracene, chrysene, aceanthrylene, acenaphthylene, acephenanthrylene, azulene, fluoranthene, fluorene, as-indacene, s-indacene, indene, phenalene, and pleiadene.

When two or three of the groups Ar¹ and Ar² together form a carbopolycyclic fused ring aromatic group in which one or more carbon atoms are each replaced by a hetero atom, preferred groups are radicals of suitable polyvalency obtained from acridine, carbazole, β-carboline, chromene, cinnoline, indole, indolizine, isobenzofuran, isochromene, isoindole, isoquinoline, naphthyridine, perimidine, phenanthridine, phenanthroline, phenazine, phthalazine, pteridine, purine, pyrrolizine, quinazoline, quinoline, quinolizine and quinoxaline.

### Substitution of Ar¹ and Ar¹—Anions and Cations

When C★ is a cation, A is preferably an electron-donating group, including —R¹ or —Z¹R¹, where R¹ and Z¹ are defined below. Preferably, R¹ is C₁₋₈hydrocarbyl, more preferably C₁₋₈alkyl, especially methyl. Z¹ is preferably O, S or NR'. R¹ may be substituted with one or more S_{ub}², but is preferably unsubstituted. When C★ is a cation, A is preferably —OMe, —SMe, —N(Me)₂ or Me. When C★ is a cation, A, when an electron-donating group, is preferably provided (especially in relation to Ar¹ or Ar² being phenyl) in a position ortho or para to C★, preferably para. Furthermore, when C★ is a cation, A, when an electron-withdrawing group (e.g. F), is preferably provided (especially in relation to Ar¹ or Ar² being phenyl) in a position meta to C★. Thus, preferred groups Ar¹ and Ar² are as follows:

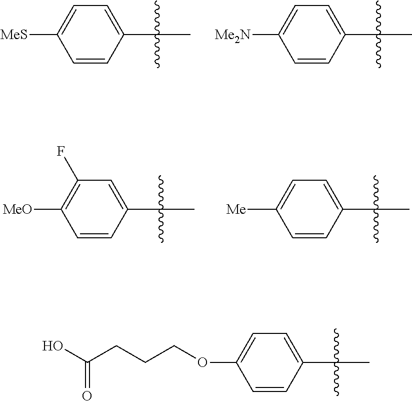

When C★ is an anion, A is preferably an electron-withdrawing group, including halogen, trihalomethyl, —NO₂, —CN, —N⁺(R¹)₂O⁻, —CO₂H, —CO₂R¹, —SO₃H, —SOR¹, —SO₂R¹, —SO₃R¹, —OC(═O)OR¹, —C(═O)H, —C(═O)R¹, —OC(═O)R¹, —C(═O)NH₂, —C(═O)NR¹₂, —N(R¹)C(═O)OR¹, —N(R¹)C(═O)NR¹₂, —OC(═O)NR¹₂, —N(R¹)C(═O)R¹, —C(═S)NR¹₂, —NR¹C(═S)R¹, —SO₂NR¹₂, —NR¹SO₂R¹, —NR¹)C(═S)NR¹₂, or —N(R¹)SO₂NR¹₂, where R¹ is defined below. When C★ is an anion, A, when an electron-withdrawing group, is preferably provided (especially in relation to Ar¹ or Ar² being phenyl) in a position ortho or para to C★, preferably para. Furthermore, when C★ is an anion, A, when an electron-donating group, is preferably provided (especially in relation to Ar¹ or Ar² being phenyl) in a position meta to C★.

The group A may also comprise one or more isotopes of the atoms making up group A (e.g. example 60), thus, as discussed in more detail below, allowing the masses of the compounds of the invention to be varied. Preferred isotopes are ¹³C, ¹⁸O and ²H. When providing a series of compounds which differ only in their masses, ¹³C and ¹⁸O are particularly preferred as ²H atoms may cause a substantial change in the chemical properties of the compound due to the kinetic isotope effect.

### Solid Supports

‘Solid supports’ for use with the invention include polymer beads, metals, resins, columns, surfaces (including porous surfaces) and plates (e.g. mass-spectrometry plates).

The solid support is preferably one suitable for use in a mass spectrometer, such that the invention can be conveniently accommodated into existing MS apparatus. Ionisation plates from mass spectrometers are thus preferred solid supports; e.g. gold, glass-coated or plastic-coated plates. Solid gold supports are particularly preferred.

Resins or columns, such as those used in affinity chromatography and the like, are particularly useful for receiving solutions of biopolymers (purified or mixtures). For example, a cellular lysate could be passed through such a column of formula (IVai), (IVaii), (IVaiii) or (IVaiv) followed by cleavage of the support to leave compounds of formula (I).

Solid supports of formulae (IVai), (IVaii), (IVaiii) or (IVaiv) will generally present exposed groups M capable of reacting with a biopolymer, B_{P}. For MS analysis, ions preferably have a predictable mass to charge (m/e) ratio. If a biopolymer reacts with more than one M group, however, then it will carry more than one positive charge once ionised, and its m/e ratio will decrease. Advantageously, therefore, the groups M are arranged such that any biopolymer molecule will covalently link with only a single group M. Consequently, each biopolymer will, on ionisation, carry a single positive charge and thus have a predictable mass to charge ratio.

Typically, the surface density of the solid supports of (IVai), (IVaii), (IVaiii) or (IVaiv) will be provided so that a biopolymer molecule can only covalently link with one group M and thus to prevent the formation of multiply derivatised biopolymers.

### Varying the Mass of Compounds of the Invention

Within the general formulae (I), (IIa), (IIIa), (IVai), (IVaii), (IVaiii), (IVaiv), (Vai), (Vaii), (Vaiii) and (Vaiv), there is much scope for variation. There is thus much scope of variation in the mass of these compounds. In some embodiments of the invention, it is preferred to use a series of two or more (e.g. 2, 3, 4, 5, 6 or more) compounds with different and defined molecular masses.

The masses of the compounds of the invention can be varied via L_{M}, Ar¹ and/or Ar². Preferably, the masses of the compounds of the invention are varied by varying A on the groups Ar¹ and/or Ar².

In this aspect of invention, compounds of the invention advantageously comprise one or more of F or I as substituents A of the groups Ar¹, Ar² or Ar³. F and I each only have one naturally occurring isotope, ¹⁹F and ¹²⁷I respectively, and thus by varying the number of F and I atoms present in the structure of the compounds, can provide a series of molecular mass labels having substantially identical shaped peaks on a mass spectrum.

Compounds of the invention may also include one or more ²H atoms, preferably as a substituent A or a part thereof of the groups L_{M}, Ar¹, Ar² or Ar³ (in particular L_{M}), in order to vary the masses of the compounds of the invention. The compounds of the invention may include isotopes of ¹³C and ¹⁸O, preferably as a substituent A or a part thereof of the groups L_{M}, Ar¹, Ar¹ or Ar³ (in particular Ar¹, Ar² or Ar³), in order to vary the masses of the compounds of the invention. Compounds comprising ²H, ¹³C and ¹⁸O may also be used to provide a series of molecular mass labels having substantially identical shaped peaks on a mass spectrum, by varying the number of ²H, ¹³C and ¹⁸O atoms present in the structure of the compounds. When providing a series of compounds which differ only in their masses, ¹³C and ¹⁸O are particularly preferred as ²H atoms may cause a substantial change in the chemical properties of the compound due to the kinetic isotope effect.

In order to increase the molecular mass of the compounds of the invention and to increase the number of available sites for substitution by A, especially F and I, one or more of Ar¹ and Ar² may be substituted by one or more dendrimer radicals of appropriate valency, either as substituent A or group L_{M}.

Preferred dendrimer radicals are the radicals obtained from the dendrimers of U.S. Pat. No. 6,455,071 and PAMAM dendrimers.

The compounds of the invention may advantageously be used in the method of analysing a biopolymer disclosed herein, in particular in a method for following a reaction involving a biopolymer, B_{P}, since the abundance of a species of may be determined by mass spectrometry by measuring the intensity of the relevant peak in an obtained mass spectrum.

Specifically, there is provided a method for analysing biopolymer B_{P}, comprising the steps of:
(i) reacting a first sample comprising biopolymer B_{P} with a compound of formula (IIa) or a solid support of formula (IVai), (IVaii), (IVaiii) or (IVaiv) at a time t₁;
(ii) reacting a second sample comprising biopolymer B_{P} with a compound of formula (IIa) or a solid support of formula (IVai), (IVaii), (IVaiii) or (IVaiv) at a later time t₂;
(iii) preparing and analysing cations of formula (I) from the first and second samples; and
(iv) comparing the results of the analysis from step (iii).

If levels of the blopolymer B_{P} decrease between times t₁ and t₂ then there will be a decrease in detected ion; if levels of the biopolymer B_{P} increase between times t₁ and t₂ then there will be an increase in detected ion. The effects of stimuli on transcription and/or translation can therefore be monitored.

Advantageously, different compounds of formula (IIa) or different solid supports of formula (IVai), (IVaii), (IVaiii) or (IVaiv) are used at different times in order to facilitate simultaneous and parallel analysis of the first and second samples. For example, if the two compounds used at times t₁ and t₂ differ only by a ¹H to ¹⁹F substitution then the relative abundance of B_{P} at the two times can be determined by comparing peaks separated by 18 units.

Advantageously, the reaction of the biopolymer with the compound of formula (IIa) or the solid support of formula (IVai), (IVaii), (IVaiii) or (IVaiv) will fix the biopolymer to prevent it reacting further and the steps of providing and analysing the cations may be carried out at a later convenient time. Alternatively, if the reaction of the biopolymer with the compound of formula (IIa) or the solid support of formula (IVai), (IVaiii) or (IVaiv) does not quench the reaction of the biopolymer being followed, a cation of formula (I) from the reaction product of step (i) or step (v) should be obtained as soon as possible after reaction of the biopolymer with the compound of formula (IIa) or the solid support of formula (IVai), (IVaii), (IVaiii) or (IVaiv).

### Compounds of Formula (IIa)

The compounds of formula (IIa) are available commercially or may be synthesised by known techniques.

Commercially available trityls, and derivatives and analogues thereof, may also be derivatised with the groups (L_{M}{M}ₚ)_{q} by known techniques. Groups (L_{M}{M}ₚ)_{q} are usually introduced into the intermediates and the compounds are then assembled using the appropriate pathways. Alternatively, the groups (L_{M}-{M}ₚ)_{q} may be added after assembly of the aromatic groups and α-carbon of the compounds.

Compounds of formula (IIa) may be synthesised analogously to the synthetic routes disclosed in PCT/GB2004/005140, Chem. Soc. Rev. (2003) 32 p. 3-13 scheme 2 and “1. introduction” last two paragraphs, WO99/60007 and EP 1 506 959. The compounds of the invention may also be synthesised by the treatment of a halide (e.g chloride) of a triarylmethyl derivative with an appropriate thiol.

### Chemical Groups

The ions of the invention are stabilised by the resonance effect of the aromatic groups Ar¹ and Ar². The term ‘C★ is a carbon atom bearing a single positive charge or a single negative charge’ therefore not only includes structures having the charge localised on the carbon atom but also resonance structures in which the charge is delocalised from the carbon atom.

The term ‘linker atom or group’ includes any divalent atom or divalent group.

The term ‘aromatic group’ includes quasi and/or pseudo-aromatic groups, e.g. cyclopropyl and cyclopropylene groups.

The term ‘halogen’ includes fluorine, chlorine, bromine and iodine.

The term ‘hydrocarbyl’ includes linear, branched or cyclic monovalent groups consisting of carbon and hydrogen. Hydrocarbyl groups thus include alkyl, alkenyl and alkynyl groups, cycloalkyl (including polycycloalkyl), cycloalkenyl and aryl groups and combinations thereof, e.g. alkylcycloalkyl, alkylpolycycloalkyl, alkylaryl, alkenylaryl, cycloalkylaryl, cycloalkenylaryl, cycloalkylalkyl, polycycloalkylalkyl, arylalkyl, arylalkenyl, arylcycloalkyl and arylcycloalkenyl groups. Preferred hydrocarbyl are C₁₋₁₄ hydrocarbyl, more preferably C₁₋₈ hydrocarbyl.

Unless indicated explicitly otherwise, where combinations of groups are referred to herein as one moiety, e.g. arylalkyl, the last mentioned group contains the atom by which the moiety is attached to the rest of the molecule.

The term ‘hydrocarbylene’ includes linear, branched or cyclic divalent groups consisting of carbon and hydrogen formally made by the removal of two hydrogen atoms from the same or different (preferably different) skeletal atoms of the group. Hydrocarbylene groups thus include alkylene, alkenylene and alkynylene groups, cycloalkylene (including polycycloalkylene), cycloalkenylene and arylene groups and combinations thereof, e.g. alkylenecycloalkylene, alkylenepolycycloalkylene, alkylenearylene, alkenylenearylene, cycloalkylenealkylene, polycycloalkylenealkylene, arylenealkylene and arylenealkenylene groups. Preferred hydrocarbylene are C₁₋₁₄ hydrocarbylene, more preferably C₁₋₈ hydrocarbylene.

The term ‘hydrocarbyloxy’ means hydrocarbyl-O—.

The terms ‘alkyl’, ‘alkylene’, ‘alkenyl’, ‘alkenylene’, ‘alkynyl’, or ‘alkynylene’ are used herein to refer to both straight, cyclic and branched chain forms. Cyclic groups include C₃₋₈ groups, preferably C₅₋₈ groups.

The term ‘alkyl’ includes monovalent saturated hydrocarbyl groups. Preferred alkyl are C₁₋₈, more preferably C₁₋₄ alkyl such as methyl, ethyl, n-propyl, i-propyl or t-butyl groups.

Preferred cycloalkyl are C₅₋₈ cycloalkyl.

The term ‘alkoxy’ means alkyl-O—.

The term ‘alkenyl’ includes monovalent hydrocarbyl groups having at least one carbon-carbon double bond and preferably no carbon-carbon triple bonds. Preferred alkenyl are C₂₋₄ alkenyl.

The term ‘alkynyl’ includes monovalent hydrocarbyl groups having at least one carbon-carbon triple bond and preferably no carbon-carbon double bonds. Preferred alkynyl are C₂₋₄ alkynyl.

The term ‘aryl’ includes monovalent aromatic groups, such as phenyl or naphthyl. In general, the aryl groups may be monocyclic or polycyclic fused ring aromatic groups. Preferred aryl are C₆-C₁₄aryl.

Other examples Of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term ‘alkylene’ includes divalent saturated hydrocarbylene groups. Preferred alkylene are C₁₋₄ alkylene such as methylene, ethylene, n-propylene, i-propylene or t-butylene groups.

Preferred cycloalkylene are C₅₋₈ cycloalkylene.

The term ‘alkenylene’ includes divalent hydrocarbylene groups having at least one carbon-carbon double bond and preferably no carbon-carbon triple bonds. Preferred alkenylene are C₂₋₄ alkenylene.

The term ‘alkynylene’ includes divalent hydrocarbylene groups having at least one carbon-carbon triple bond and preferably no carbon-carbon double bonds. Preferred alkynylene are C₂₋₄ alkynylene.

The term ‘arylene’ includes divalent aromatic groups, such phenylene or naphthylene. In general, the arylene groups may be monocyclic or polycyclic fused ring aromatic groups. Preferred arylene are C₆-C₁₄arylene.

Other examples of arylene groups are divalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term ‘heterohydrocarbyl’ includes hydrocarbyl groups in which up to three carbon atoms, preferably up to two carbon atoms, more preferably one carbon atom, are each replaced independently by O, S, Se or N, preferably O, S or N. Heterohydrocarbyl groups thus include heteroalkyl, heteroalkenyl and heteroalkynyl groups, cycloheteroalkyl (including polycycloheteroalkyl), cycloheteroalkenyl and heteroaryl groups and combinations thereof, e.g. heteroalkylcycloalkyl, alkylcycloheteroalkyl, heteroalkylpolycycloalkyl, alkylpolycycloheteroalkyl, heteroalkylaryl, alkylheteroaryl, heteroalkenylaryl, alkenylheteroaryl, cycloheteroalkylaryl, cycloalkylheteroaryl, heterocycloalkenylaryl, cycloalkenylheteroaryl, cycloalkylheteroalkyl, cycloheteroalkylalkyl, polycycloalkylheteroalkyl, polycycloheteroalkylalkyl, arylheteroalkyl, heteroarylalkyl, arylheteroalkenyl, heteroarylalkenyl, arylcycloheteroalkyl, heteroarylcycloalkyl, arylheterocycloalkenyl and heteroarylcycloalkenyl groups. The heterohydrocarbyl groups may be attached to the remainder of the compound by any carbon or hetero (e.g. nitrogen) atom.

The term ‘heterohydrocarbylene’ includes hydrocarbylene groups in which up to three carbon atoms, preferably up to two carbon atoms, more preferably one carbon atom, are each replaced independently by O, S, Se or N, preferably O, S or N. Heterohydrocarbylene groups thus include heteroalkylene, heteroalkenylene and heteroalkynylene groups, cycloheteroalkylene (including polycycloheteroalkylene), cycloheteroalkenylene and heteroarylene groups and combinations thereof, e.g. heteroalkylenecycloalkylene, alkylenecycloheteroalkylene, heteroalkylenepolycycloalkylene, alkylenepolycyclobeteroalkylene, heteroalkylenearylene, alkyleneheteroarylene, heteroalkenylenearylene, alkenyleneheteroarylene, cycloalkyleneheteroalkylene, cycloheteroalkylenealkylene, polycycloalkyleneheteroalkylene, polycycloheteroalkylenealkylene, aryleneheteroalkylene, heteroarylenealkylene, aryleneheteroalkenylene, heteroarylenealkenylene groups. The heterohydrocarbylene groups may be attached to the remainder of the compound by any carbon or hetero (e.g. nitrogen) atom.

Where reference is made to a carbon atom of a hydrocarbyl or other group being replaced by an O, S, Se or N atom, what is intended is that:

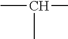

is replaced by

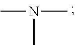

—CH═ is replaced by —N═; or
—CH₂— is replaced by —O—, —S— or —Se—.

The term ‘heteroalkyl’ includes alkyl groups in which up to three carbon atoms, preferably up to two carbon atoms, more preferably one carbon atom, are each replaced independently by O, S, Se or N, preferably O, S or N.

The term ‘heteroalkenyl’ includes alkenyl groups in which up to three carbon atoms, preferably up to two carbon atoms, more preferably one carbon atom, are each replaced independently by O, S, Se or N, preferably O, S or N.

The term ‘heteroalkynyl’ includes alkynyl groups in which up to three carbon atoms, preferably up to two carbon atoms, more preferably one carbon atom, are each replaced independently by O, S, Se or N, preferably O, S or N.

The term ‘heteroaryl’ includes aryl groups in which up to three carbon atoms, preferably up to two carbon atoms, more preferably one carbon atom, are each replaced independently by O, S, Se or N, preferably O, S or N. Preferred heteroaryl are C₅₋₁₄heteroaryl. Examples of heteroaryl are pyridyl, pyrrolyl, thienyl or furyl.

Other examples of heteroaryl groups are monovalent derivatives of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene. Preferred heteroaryl groups are five- and six-membered monovalent derivatives, such as the monovalent derivatives of furan, imidazole, isothiazole, isoxazole, pyran, pyrazine, pyrazole, pyridazine, pyridine, pyrimidine, pyrrole, pyrrolizine and thiophene. The five-membered monovalent derivatives are particularly preferred, i.e. the monovalent derivatives of furan, imidazole, isothiazole, isoxazole, pyrazole, pyrrole and thiophene.

The term ‘heteroancylene’ includes alkylene groups in which up to three carbon atoms, preferably up to two carbon atoms, more preferably one carbon atom, are each replaced independently by O, S, Se or N, preferably O, S or N.

The term ‘heteroalkenylene’ includes alkenylene groups in which up to three carbon atoms, preferably up to two carbon atoms, more preferably one carbon atom, are each replaced independently by O, S, Se or N, preferably O, S or N.

The term ‘heteroalkynylene’ include alkynylene groups in which up to three carbon atoms, preferably up to two carbon atoms, more preferably one carbon atom, are each replaced independently by O, S, Se or N, preferably O, S or N.

The term ‘heteroarylene’ includes arylene groups in which up to three carbon atoms, preferably up to two carbon atoms, more preferably one carbon atom, are each replaced independently by O, S, Se or N, preferably O, S or N. Preferred heteroarylene are C₅₋₁₄heteroarylene. Examples of heteroarylene are pyridylene, pyrrolylene, thienylene or furylene.

Other examples of heteroarylene groups are divalent derivatives (where the valency is adapted to accommodate the q instances of the linker L_{M}) of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene. Preferred heteroarylene groups are five- and six-membered divalent derivatives, such as the divalent derivatives of furan, imidazole, isothiazole, isoxazole, pyran, pyrazine, pyrazole, pyridazine, pyridine, pyrimidine, pyrrole, pyrrolizine and thiophene. The five-membered divalent derivatives are particularly preferred, i.e. the divalent derivatives of furan, imidazole, isothiazole, isoxazole, pyrazole, pyrrole and thiophene.

### Substitution

A is independently a substituent, preferably a substituent S_{ub}¹. Alternatively, A may be ²H.

S_{ub}¹ is independently halogen, trihalomethyl, —NO₂, —CN, —N⁺(R¹)₂O⁻, —CO₂H, —CO₂R¹, —SO₃H, —SOR¹, —SO₂R¹, —SO₃R¹, —OC(═O)OR¹, —C(═O)H, —C(═O)R¹, —OC(═O)R¹, —NR¹₂, —C(═O)NH₂, —C(═O)NR¹₂, —N(R¹)C(═O)OR¹, —N(R¹)C(O)NR¹₂, —OC(═O)NR¹₂, —N(R¹)C(═O)R¹, —C(═S)NR¹₂, —NR¹C(═S)R¹, —SO₂NR¹₂, —NR¹SO₂R¹, —N(R¹)C(═S)NR¹₂, —N(R¹)SO₂NR¹₂, —R¹ or —Z¹R¹.

Z¹ is O, S, Se or NR¹.

R¹ is independently H, C₁₋₈hydrocarbyl, C₁₋₈hydrocarbyl substituted with one or more S_{ub}², C₁₋₈heterohydrocarbyl or C₁₋₈heterohydrocarbyl substituted with one or more S_{ub}².

S_{ub}² is independently halogen, trihalomethyl, —NO₂, —CN, —N(C₁₋₆alkyl)₂O⁻, —CO₂H, —SO₃H, —SO₃C₁₋₆alkyl, —OC(═O)OC₁₋₆alkyl, —C(═O)H, —C(═O)C₁₋₆alkyl, —OC(═O)C₁₋₆alkyl, —N(C₁₋₆alkyl)₂), —C(═O)NH₂, —C(═O)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆alkyl)C(═S)N(C₁₋₆alkyl)₂, —N(C₁₋₆alkyl)SO₂N(C₁₋₆alkyl)₂, C₁₋₆alkyl or —Z¹C₁₋₆alkyl.

Where reference is made to a substituted group, the substituents are preferably from 1 to 5 in number, most preferably 1.

However, molecular mass labels of the invention will generally comprise 1 or more, typically between 1 and 100 (e.g. 1 to 50, preferably 1 to 20) substituents S_{ub}¹ or S_{ub}², typically F or I, in order to vary the masses of the molecular mass labels.

Preferred examples of substituent A are shown in FIG. 14.

### Miscellaneous

A may optionally be a monovalent dendrimer radical or a monovalent dendrimer radical substituted with one or more substituents S_{ub}¹.

### General

The term “comprising” means “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

### Tables

TABLE 1

| Formula | Structure |
|---|---|
| Formula (I) | 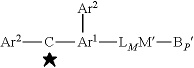 |
| Formula (IIa) | 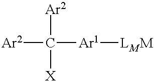 |
| Formula (IIIa) | 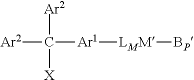 |
| Formula (IVai) | 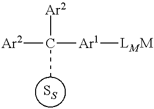 |
| Formula (IVaii) | 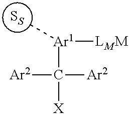 |
| Formula (IVaiii) | 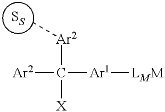 |
| Formula (IVaiv) | 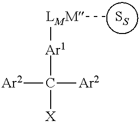 |
| Formula (Vai) | 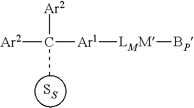 |
| Formula (Vaii) | 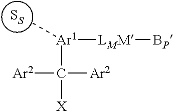 |
| Formula (Vaiii) | 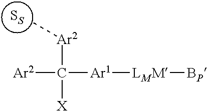 |
| Formula (Vaiv) | 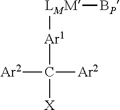 |

n = 2,
m = 1,
p = 1 and
q = 1

### BRIEF DESCRIPTION OF THE DRAWINGS

FIG. 1 shows the arrangement of the TLC plates in example 2.

FIG. 2 shows the evolution of the trityl compounds of example 2 in 40% AcOH.

FIG. 3 shows the evolution of the trityl compounds of example 2 in 80% AcOH.

FIG. 4 shows the stability of trimethoxytrityl (TMTt) compounds to standard acid solution used in DNA automatic synthesis after 1 min, 5 min and 18 h.

FIG. 5 shows the stability of aliphatic TMTt thioether against 5%, 10% and 20% TFA solutions in MeOH, water and THF.

FIGS. 6-9 show spectra of (MA)LDI MS analyses of TMTt ethers and thioethers.

FIG. 10 shows the arrangement of the TLC plates in example 4.

FIG. 11 shows the TLC plates used for monitoring the degree of cleavage of the trityl-heteroatom (O or S) bond of the compounds of example 4 in DCA deblock solution and 40% and 80% AcOH solutions.

FIG. 12 shows the TLC plates used for monitoring the degree of cleavage of the trityl-heteroatom (O or S) bond of the compounds of example 4 in 5%, 10% and 20% TFA solutions.

FIGS. 13-15 show spectra of (MA)LDI MS analyses of the compounds of example 5.

FIG. 16 shows the spotting pattern used on the gold plate of example 6.

FIG. 17 shows the MALDI MS analysis (using DHB matrix) of the trityl ether and trityl thioether of example 6.

FIG. 18 shows the LDI MS analysis (without matrix) of the trityl ether and trityl thioether of example 6.

FIGS. 19A and 19B show preferred examples of group L_{M}.

FIGS. 20A and 20B show preferred examples of group M.

FIGS. 21A and 21B show preferred examples of groups Ar¹ and Ar².

FIG. 22 shows preferred examples of substituent group A.

### MODES FOR CARRYING OUT THE INVENTION

### Example 1

### Synthesis

Trityl ethers and thioethers were obtained by the treatment of monomethoxy (MMTt) or trimethoxy trityl (TMTt) chloride with the corresponding alcohols or thiols:

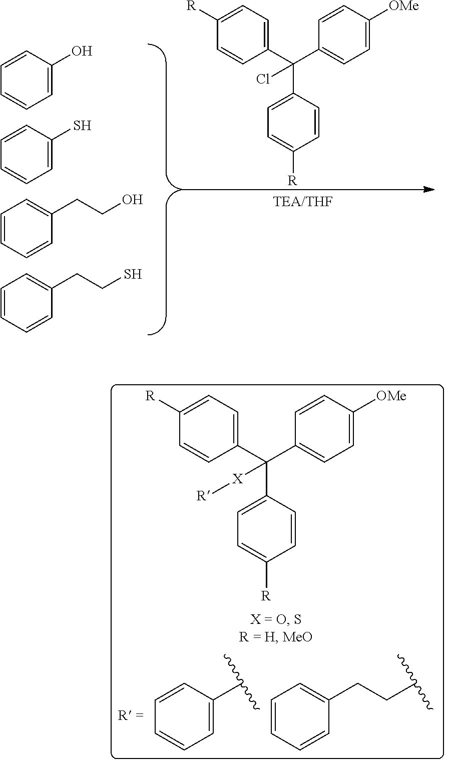

### Example 2

### Comparison of Acid Stability of Trityl Ethers and Trityl Thioethers

In order to assess the acid-stability of the oxygen-trityl bond versus the sulphur-trityl bond equivalent trityl ethers and thioethers were subjected through identical acidic conditions and the time evolution of the trityl-heteroatom bond was monitored. Since they can display different behaviour, both aliphatic and aromatic trityl ethers and thioethers were monitored. The following compounds were analysed:

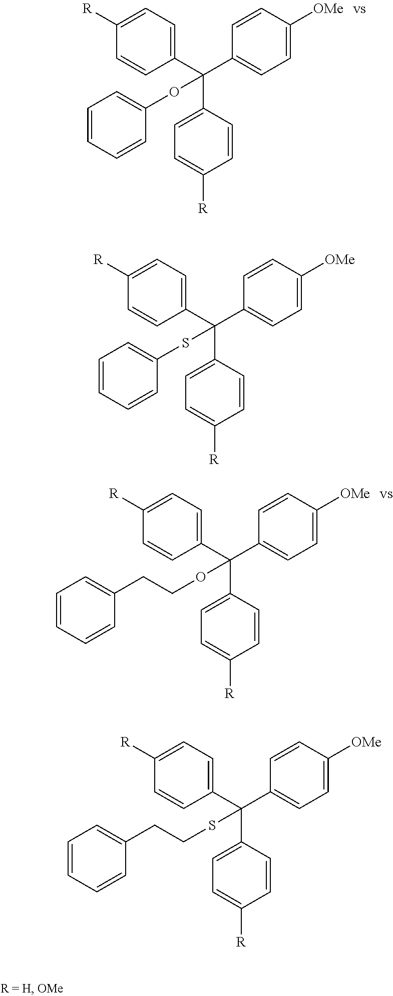

Because of its simplicity, it was decided that the most appropriate method to monitor the evolution of the trityl ethers and thioethers in acidic conditions would be thin layer chromatography (TLC).

### Experimental

1×10⁻² M solutions of the trityl ethers and thioethers to be compared were prepared with THF solvent. The following solutions were then prepared:

| solution | 1 | 2 | 3 | 4 |
|---|---|---|---|---|
| compound | 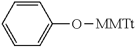 | 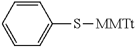 | 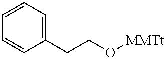 | 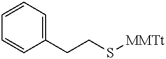 |
| Code | 23.32 | 23.21 | 23.16 | 23.19 |
| MW | 366.5 | 382.5 | 394.5 | 410.6 |
| Weight (mg) | 21.1 | 21.4 | 22.0 | 22.6 |
| vol | 5.757 | 5.595 | 5.577 | 5.504 |
| solution | 5 | 6 | 7 | 8 |
| compound | 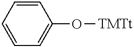 | 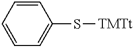 | 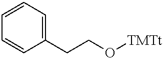 | 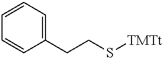 |
| code | 23.17 | 23.20 | 23.11 | 23.18 |
| MW | 426.5 | 442.6 | 454.56 | 470.6 |
| weight (mg) | 14.3 | 22.7 | 25.0 | 28.7 |
| vol | 3.353 | 5.130 | 5.500 | 6.099 |

MMTt=monomethoxytrityl; TMTt=trimethoxytrityl

The solutions were tested in both 80% and 40% acetic acid by dissolving 100 μL of the solutions above in 400 μL of 100% and 50% AcOH, respectively.

The solutions of the trityl ethers and thioethers in acetic acid were spotted in a TLC plate, and the plate run using a mixture of hexane:ethyl acetate; 4:1, with a few drops of diisopropylethylamine (DIEA).

### Results

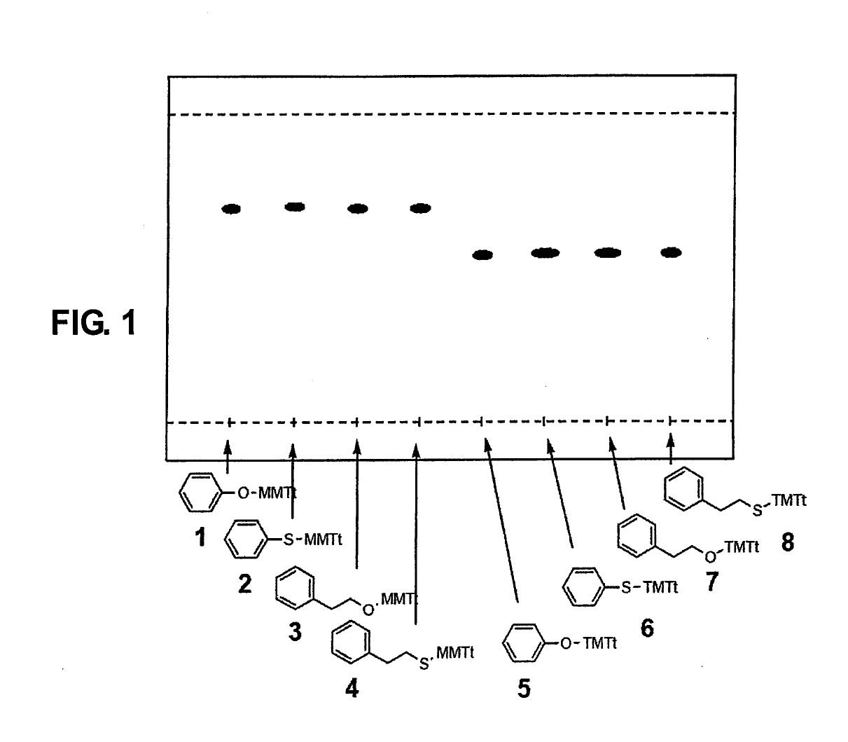

The TLC plates were examined by UV and by exposing to trifluoroacetic acid (TFA) vapours. The evolution of hydrolysis of the trityl-heteroatom bond was then visually evaluated. The arrangement of the solutions on the TLC plate is shown in FIG. 1.

### 40% AcOH Solutions

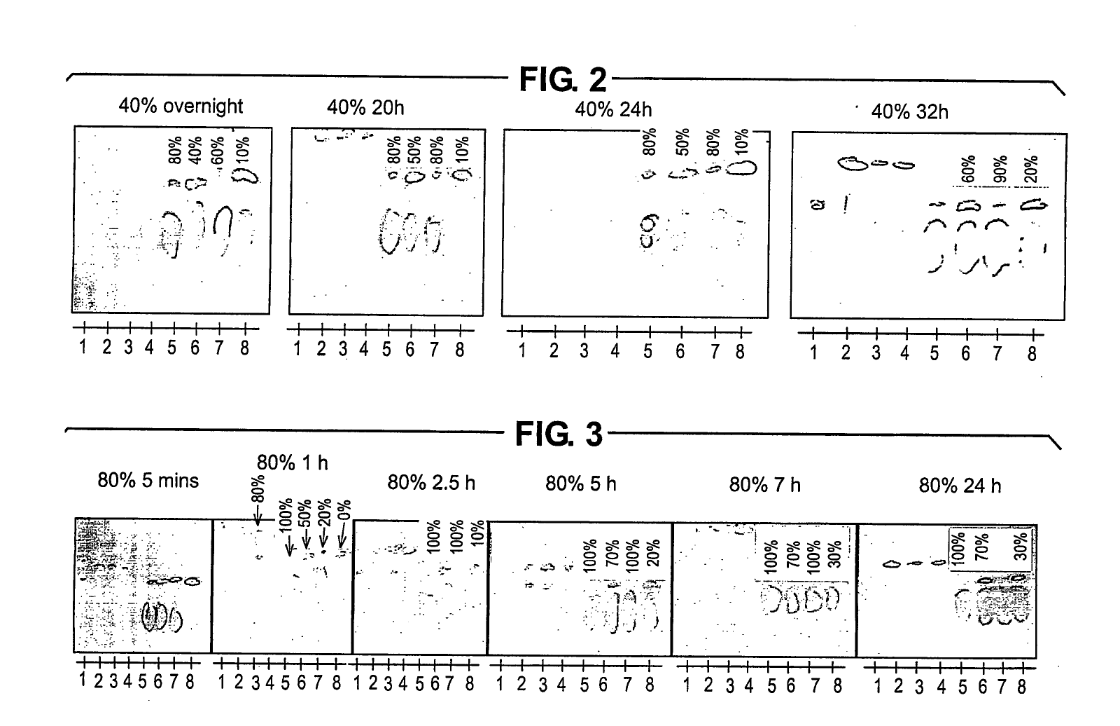

The evolution of the trityl compounds in 40% AcOH can be followed in the TLC plates shown in FIG. 2.

MMTt derivatives were stable to 40% AcOH whereas TMTt compounds were gradually cleaved. The following pattern in stability was found:

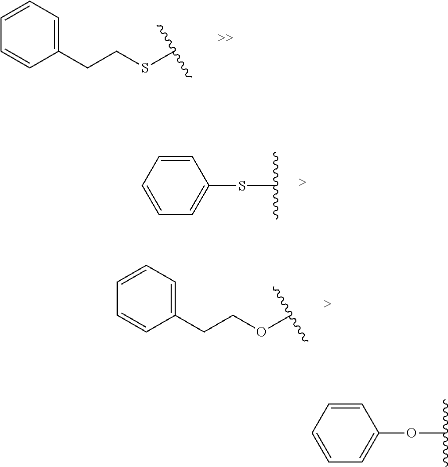

The trityl-sulphur bond is clearly more resistant to the acidic conditions than an equivalent trityl-oxygen bond. Aliphatic thioethers or ethers are more resistant than their aromatic analogues.

### 80% AcOH Solutions

The evolution of the trityl compounds in 80% AcOH can be followed in the TLC plates shown in FIG. 3.

The hydrolysis was significantly faster under these conditions, although the same pattern of stability could be observed. The stability of the aliphatic sulphur -TMTt bond was remarkably good, and after 1 h the compound remained practically without being cleaved. Even after 24 h, at least 50% of the starting thioether remained in solution.

### Further Experiments

Given that the TMTt-sulphur bond was reasonably stable to acetic acid, TMTt compounds were tested against different acid solutions.

### ABI (Applied Biosystems Inc) Acid

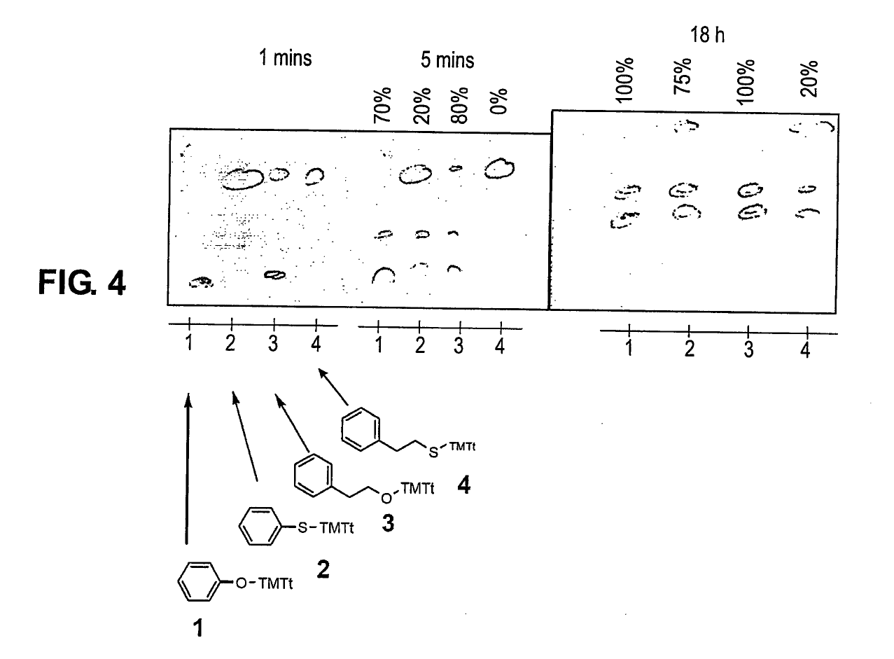

The stability of TMTt compounds to standard acid solution used in DNA automatic synthesis (‘ABI acid’) was evaluated. The results after 1 min, 5 min and 18 h are shown in FIG. 4.

TMTt ethers and thiophenyl were readily cleaved. However, the aliphatic TMTt thioether was again outstandingly resistant to the acidic conditions, and even after 18 h, most of the starting material remained uncleaved.

### Aliphatic TMTt Thioether in TFA Solutions

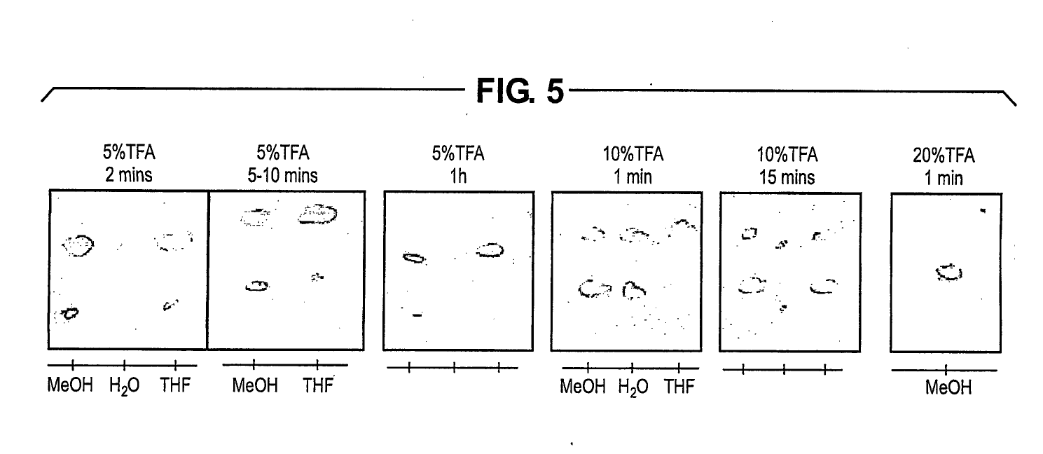

The aliphatic TMTt thioether was tested against 5%, 10% and 20% TFA solutions in MeOH, water and THF. The results are shown in FIG. 5.

Under the test conditions (room temperature, 100 μL of trityl solution in 5004, of acid solution) the trityl-sulphur bond was practically resistant to the 5% TFA solution, it was partially cleaved in 10% TFA and it was instantaneously cleaved in 20% TFA.

There was not an appreciable difference between the use of THF or MeOH as a solvent. However, the aqueous solution behaviour deserves a comment.

When the trityl solution was added to the aqueous 5% TFA solution the thioether crashed out of solution. After hours the precipitate started to disappear. When 10% TFA was used, the solution acquired an instant orange colour and no precipitate was generated. This is an indication that the trityl thioether is temporally resistant to 5% aqueous TFA, whereas 10% aqueous TFA is strong enough to cleave the thioether, liberating the trimethoxy tritylium cation and the free sulphide.

### Example 3

### (MA)LDI TOF MS Experiments

Trimethoxytrityl thioethers are more slowly cleaved under acidic conditions than the corresponding trimethoxytrityl ethers. However, taking into account the application of the solid support immobilised trimethoxy trityl as enhancers for the analysis of biopolymers, the behaviour of the sulphur-linked trityl tags under (MA)LDI conditions was verified.

Equimolar solutions of trimethoxytrityl ethers and thioethers were submitted for (MA)LDI analysis. The objective of the experiment was to obtain an estimate of the behaviour of both trimethoxytrityl ethers and thioethers under LDI MS spectrometry, both with and without the assistance of matrix.

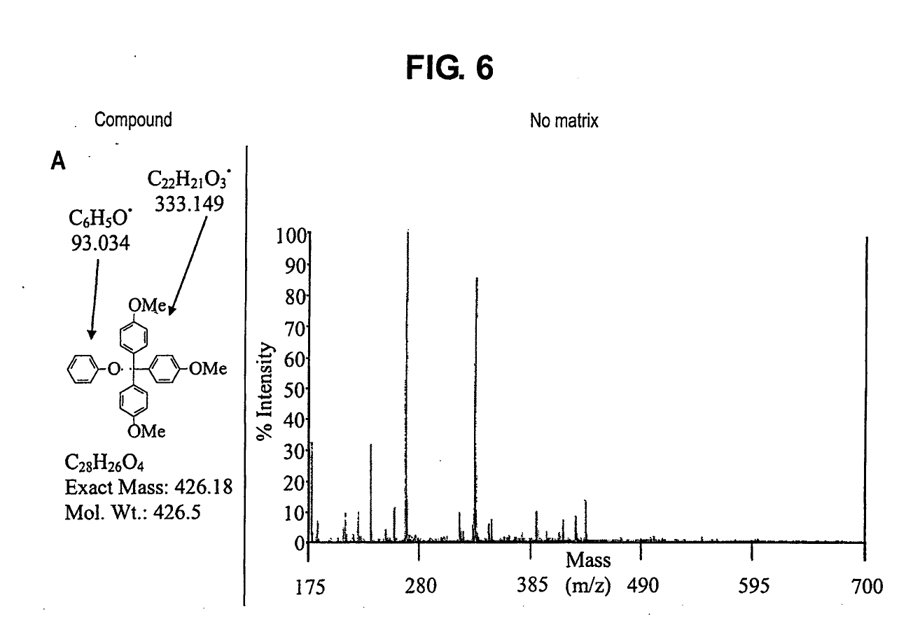

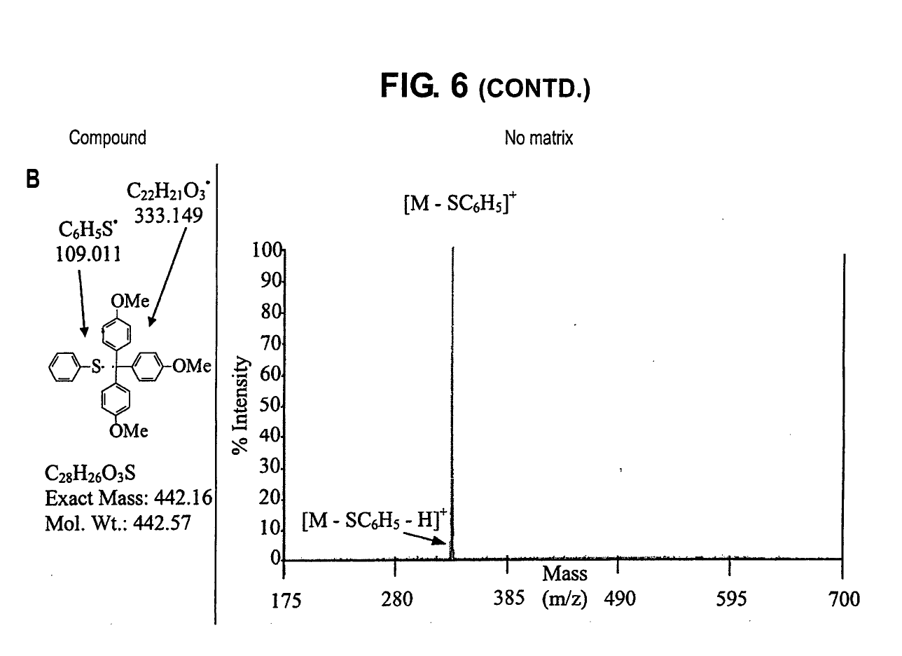

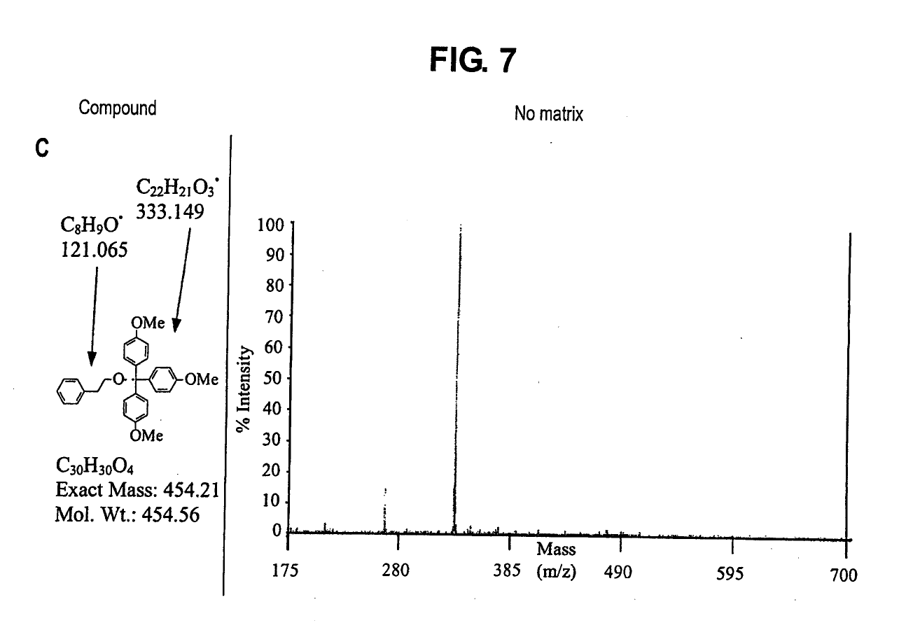

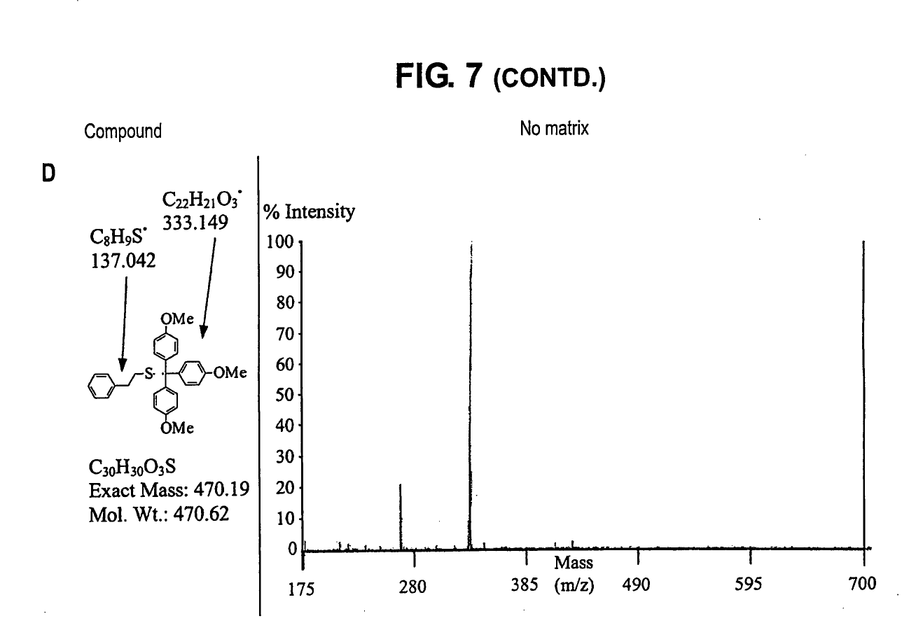

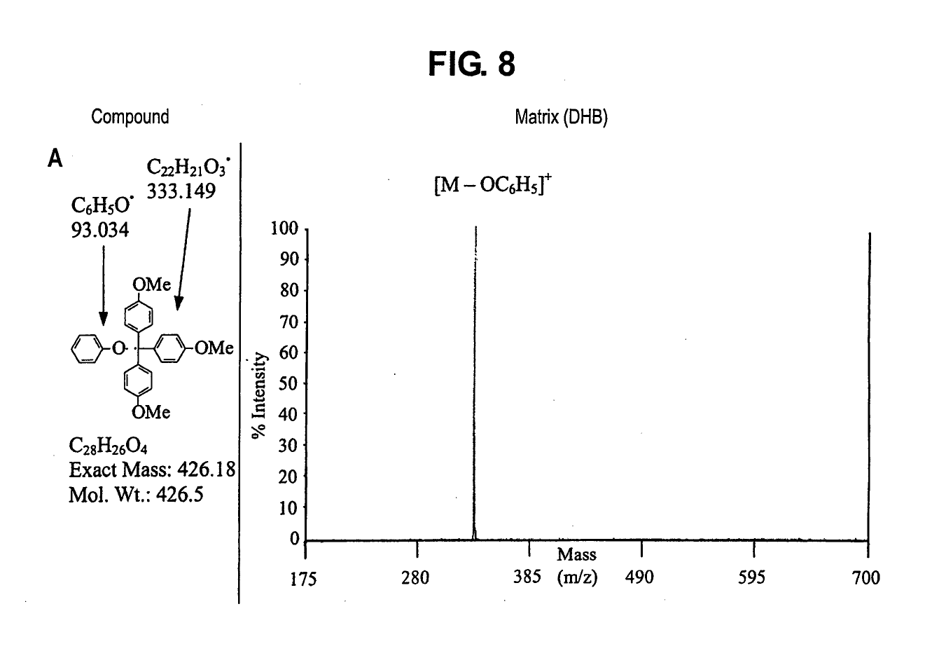

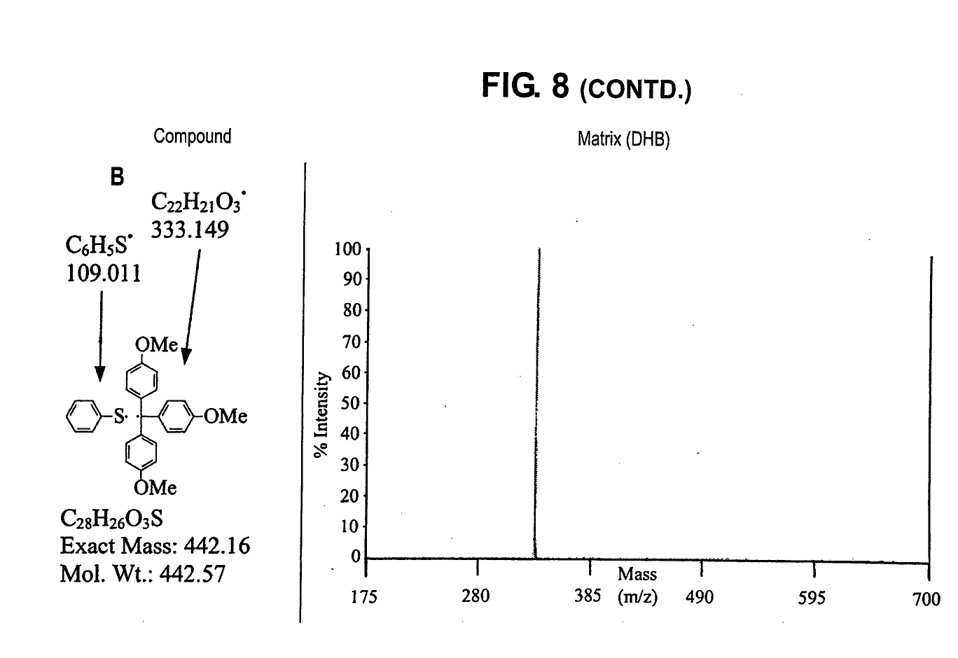

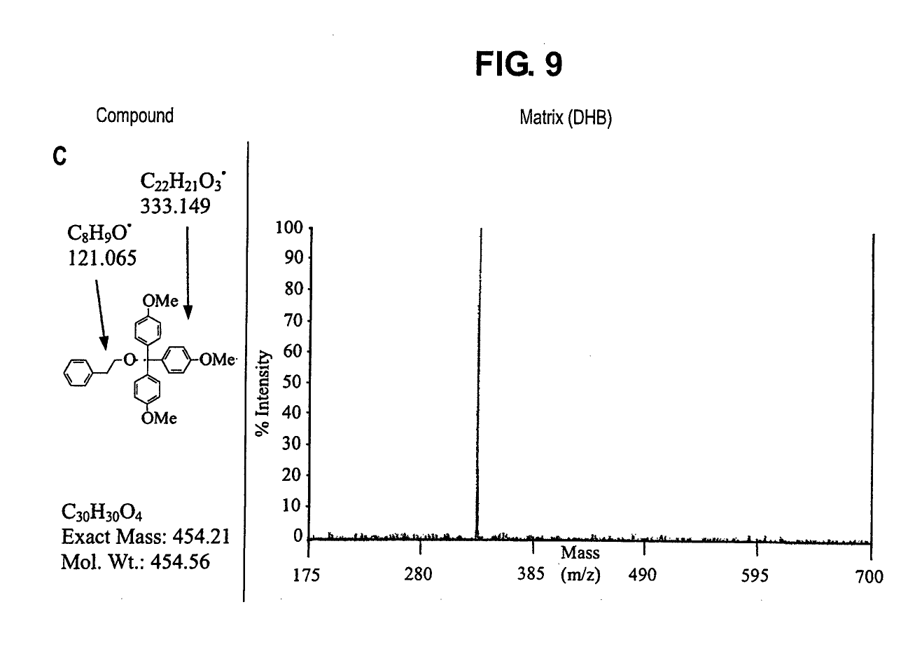

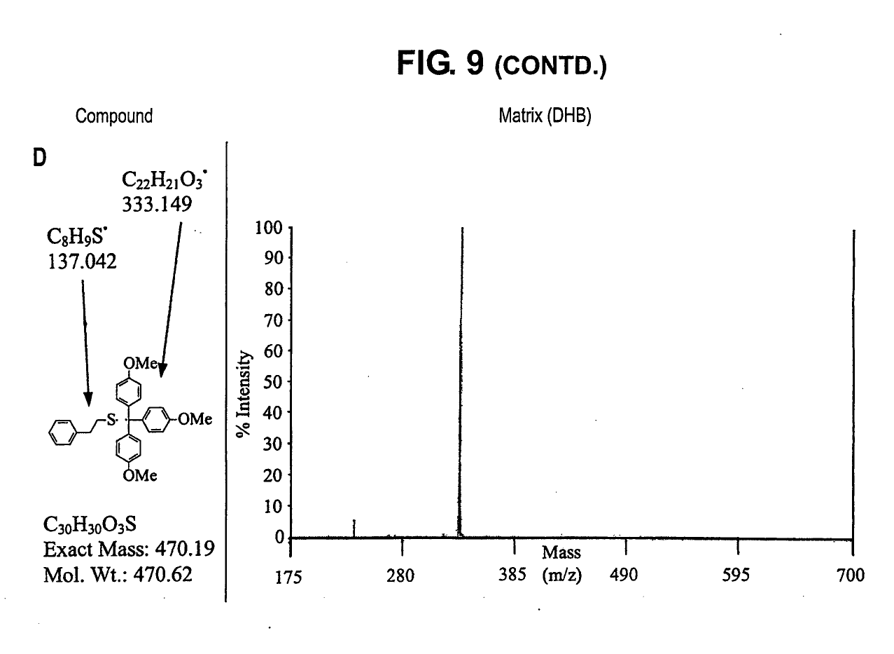

The resulting spectra are shown in FIGS. 6-9.

In all the cases, trimethoxytritylium cations from thioether samples produced peaks of a higher absolute intensity than cations from their counterpart ethers. In addition, when the samples were run without the assistance of matrix, the spectra from thioethers were evidently cleaner. This would appear to be an indication of a neater and more efficient liberation of the trityl tag in the thioethers.

### Example 4

### Comparison of Dimethoxytrityl-O (DMTt-O) and Trimethoxytrityl-S (TMTt-S)

From the previous experiments, it is evident that tritylated thiols are significantly more stable than their corresponding alcohols.

However, in order to establish the behaviour of benzyl thiol and to compare how alkyloxy-DMTts compare with mercapto-TMTts, the following experiment was carried out.

Alkyloxy-DMTt was compared against the three variations of mercapto-TMTt systems: arylthio, benzylthio and alkylthio. In this manner, the relative stability of the tritylated thiols could be classified at the time that alkoxy-DMTt was compared against the collection of mercapto-TMTts.

The following new model compounds were synthesised using standard protocols:

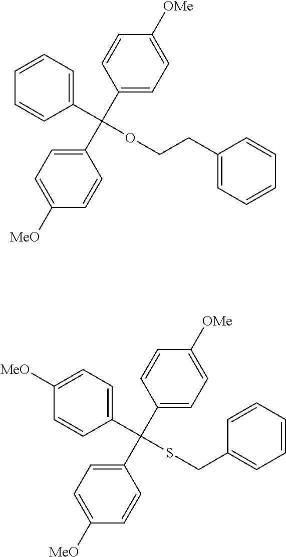

### Experimental

1×10⁻² M solutions of the compared trityl ethers and thioethers were prepared using THF as a solvent.

The following table shows the preparation of the solutions used:

| solution | 1 | 2 | 3 | 4 |
|---|---|---|---|---|
| compound | 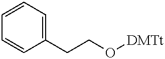 | 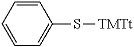 | 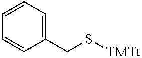 | 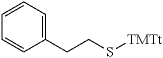 |
| Code | 23.44 | 23.20 | 23.42 | 23.18 |
| MW | 424.6 | 442.6 | 456.6 | 470.6 |
| Weight (mg) | 36.7 | 33.9 | 23.5 | 26.1 |
| vol | 6.453 | 7.659 | 5.147 | 5.546 |

In order to obtain a comprehensive picture of the process, the acidolysis of the above solutions was evaluated in 6 different acidic solutions: dichloroacetic acid (DCA) deblock solution, 80% AcOH, 40% AcOH, 5% TFA, 10% TFA and 20% TFA.

In a typical experiment, the solutions of the trityl ethers and thioethers in the series of acidic solutions were capillary spotted in a TLC plate after certain intervals, and the plate run using a mixture of hexane:ethyl acetate; 4:1, with a few drops of DIEA.

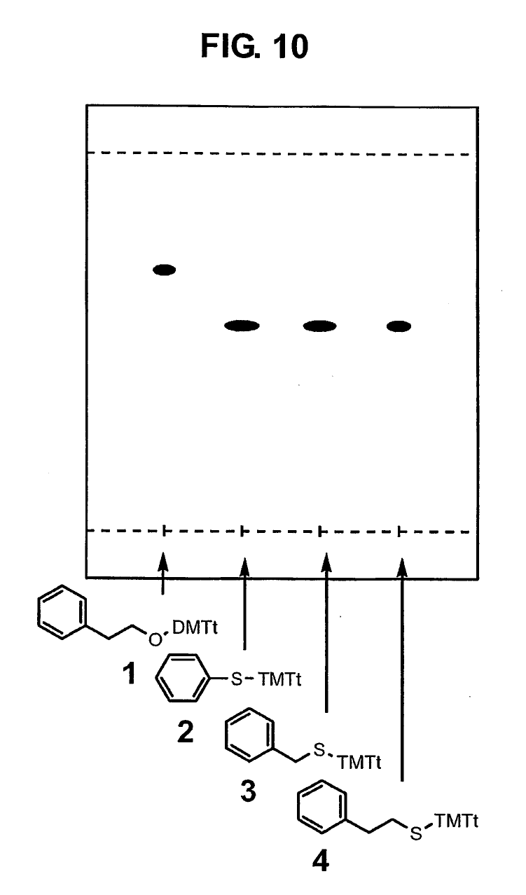

FIG. 10 shows the used layout of a TLC analysis.

The TLC plates were examined by UV and by exposing to TFA vapours. The evolution of hydrolysis of the trityl-heteroatom bond was then visually evaluated.

Analysis was carried out after controlled periods of time to determine the evolution of the trityl ethers and thioethers in the acidic solutions. Times (min): 5, 10, 20, 60, 100, 180, 240, 390, and 1440.

### Results

### General Remarks

Alkyl and benzyl mercapto-TMTts are the most resistant species to the acid solutions. The stability of both compounds is similar and significantly higher than the stability of the alkoxy-DMTt and arylmercapto-TMTt.

### DCA Deblock Solution and 40% and 80% AcOH Solutions

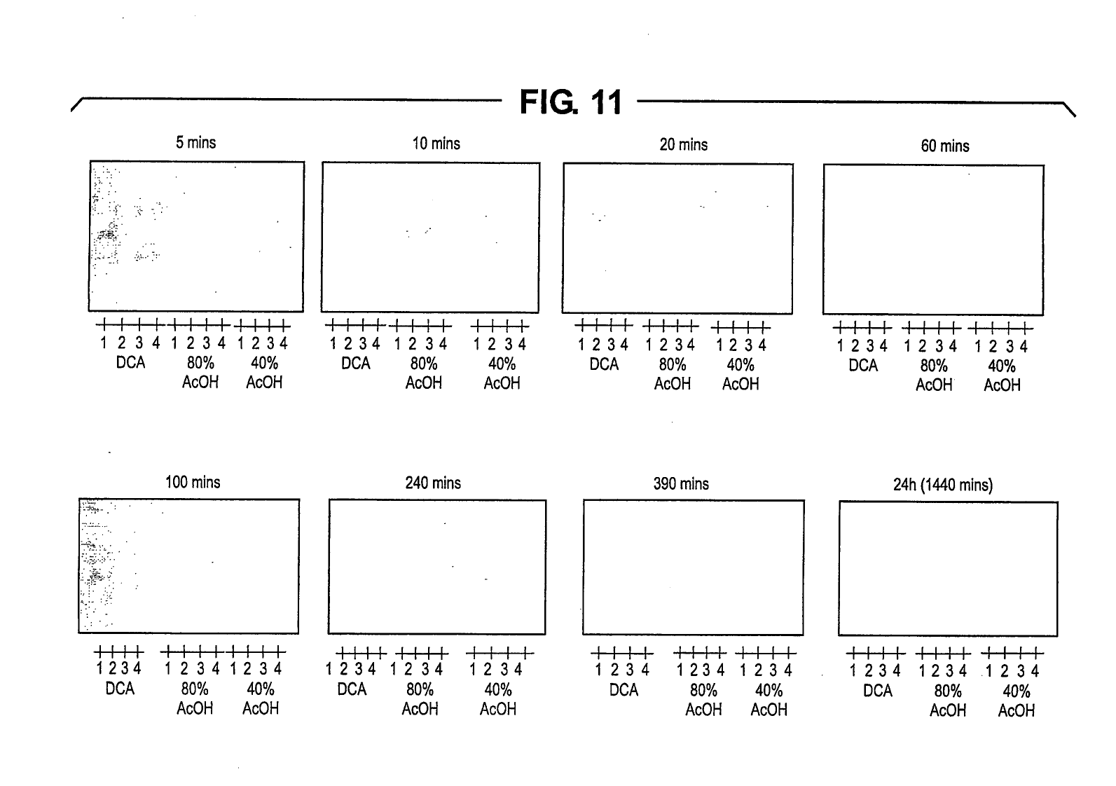

FIG. 11 shows the TLC plates used for monitoring the cleavage of the trityl-heteroatom (O or S) bond.

AlkoxyDMTt compound was practically instantaneously hydrolised by the deblock solution. ThiophenylTMTt was notably more resistant to this acidic solution. After 1 h the sulphur compound was hydrolysed in ca 80%.

Alkylmercapto and benzylmercapto-TMTts were slowly deprotected, with an approximate 50% evolution after 24 h.

Once more, two clearly different behaviours could be observed in acetic acid solutions. Alkylmercapto and benzylmercapto TMTts remained practically intact in the first 6 hours and the deprotection was just detectable after 24 h (˜20%).

The deprotection of alkoxyDMTt and thiophenylTMTt was faster. In 80% AcOH, both compounds reached a ˜50% of deprotection progress after 2 h. In 40% AcOH the 50% of deprotection progress was reached after ˜24 h.

### 5%, 10% and 20% TFA Solutions

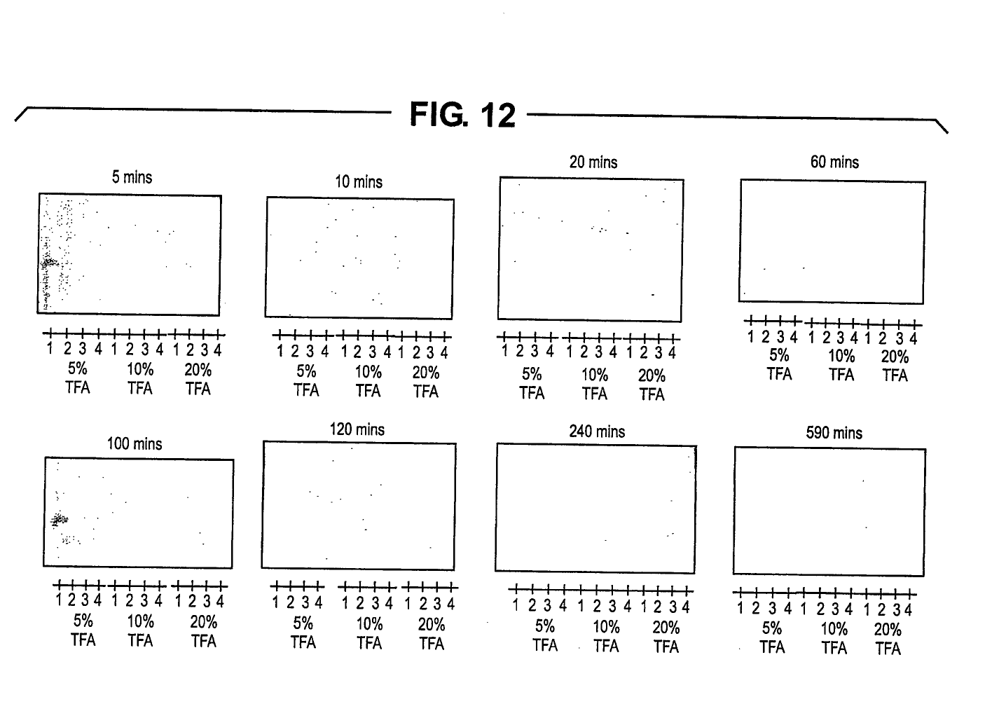

FIG. 12 shows the TLC plates used for monitoring the cleavage of the trityl-heteroatom (O or S) bond.

TFA solutions deprotected almost instantaneously the DMTt compound. ThiophenylTMTt was also deprotected quickly (in 5% TFA the deprotection was completed in 15 min).

Consistently with previous experiments, alkylmercapto and benzylmercapto TMTts were surprisingly resistant to TFA solution. In all three TFA solutions the deprotection was below 50% after 1 h, above 50% in 2 h and ca 80% after 24 h.

### Example 5

### (MA)LDI TOP MS Experiments

Trimethoxytrityl thioethers are more stable to acidic conditions than alkylic dimethoxytrityl ethers. In order to establish whether, despite its higher acid stability, TMTt thioethers would still give a higher response in (MA)LDI than DMTt ethers, the following experiments were carried out.

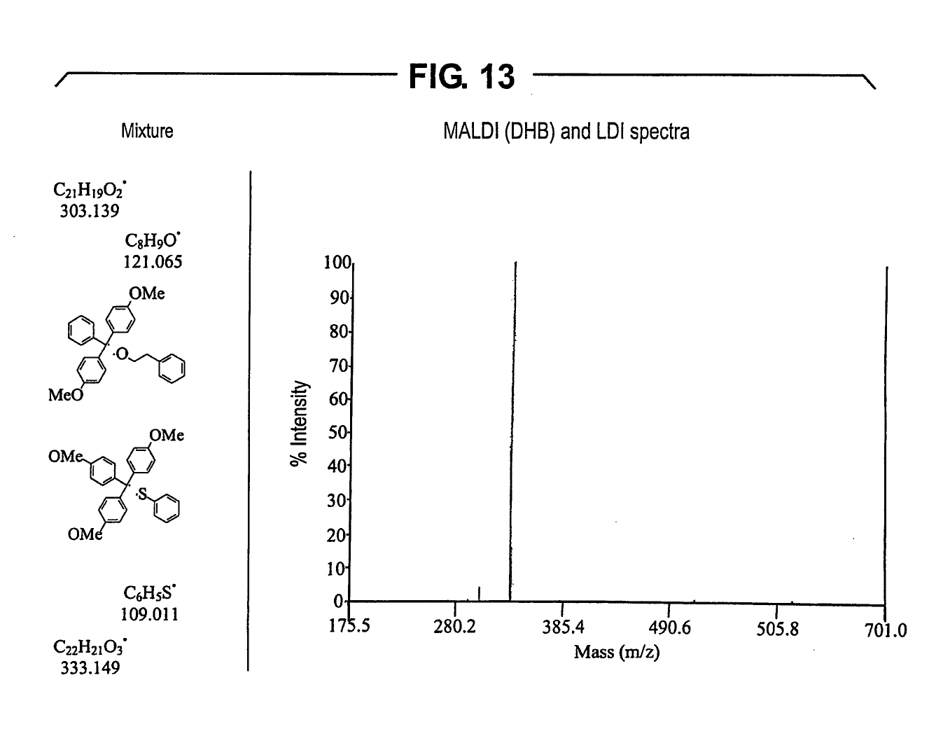

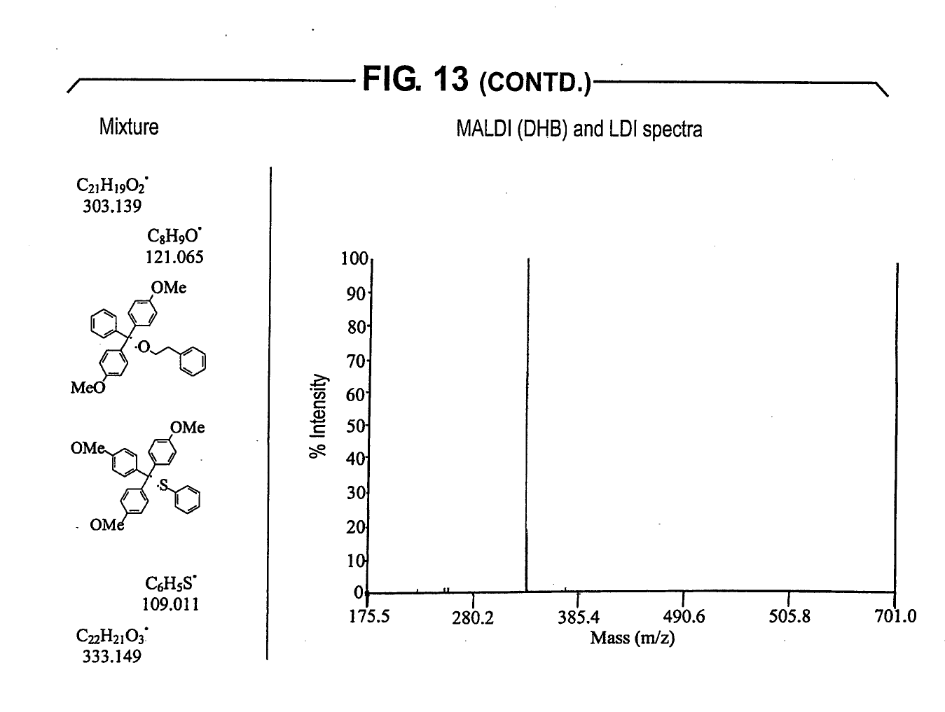

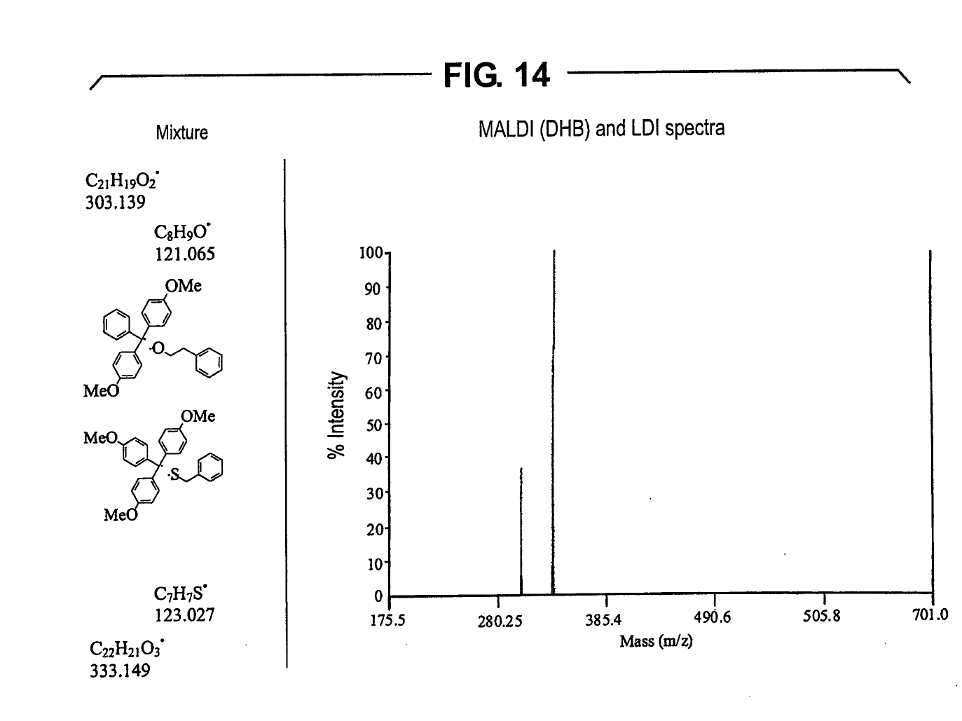

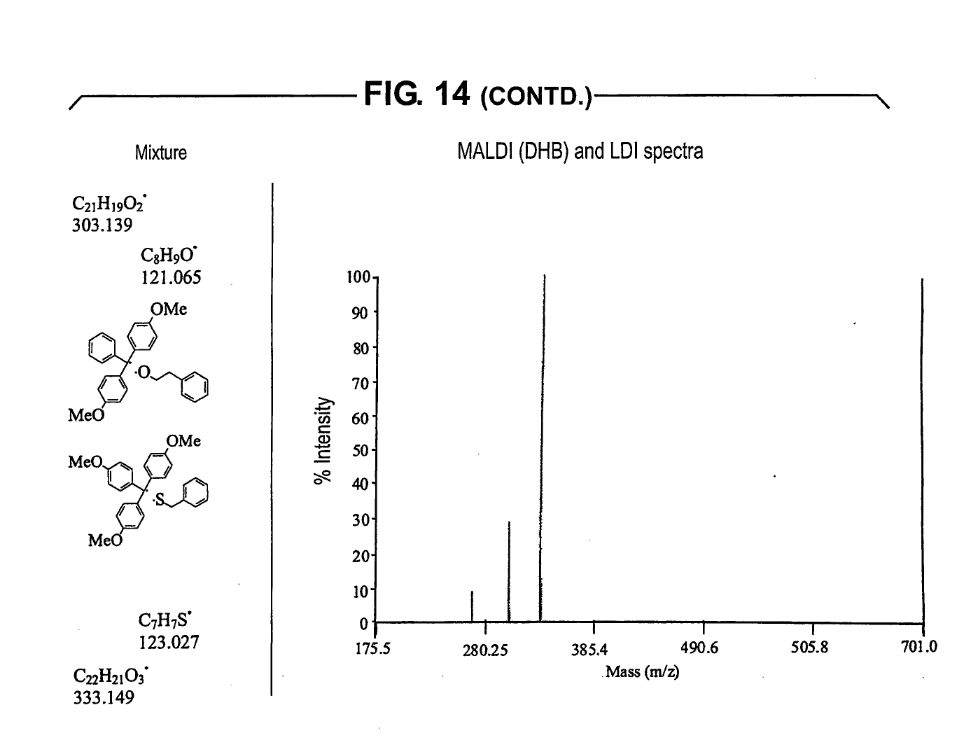

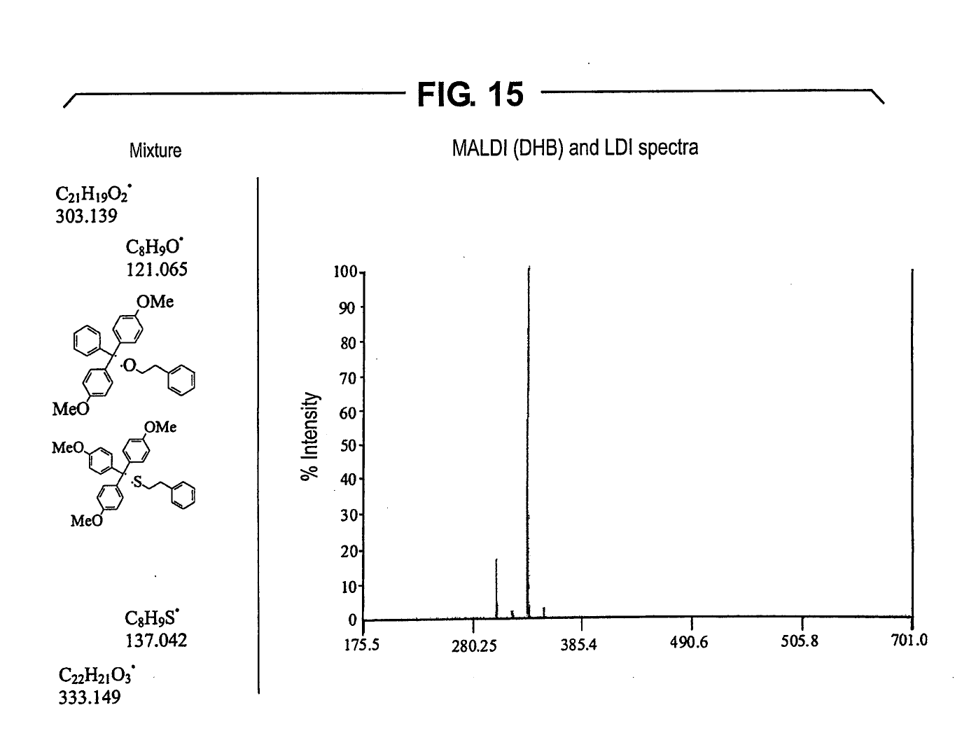

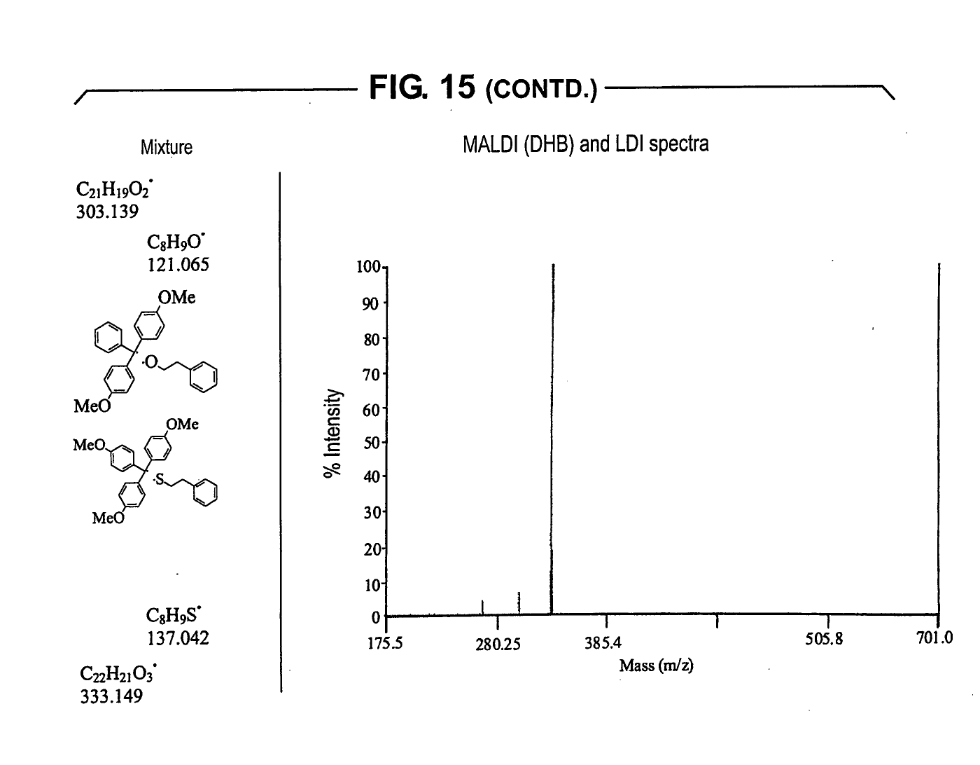

A series of binary equimolecular mixtures of alkylic DMTt ether with arylic, benzylic and alkylic TMTt thioethers, respectively, were prepared and analysed by MS with and without matrix. The results are shown in FIGS. 13, 14 and 15.

The results are conclusive. Because TMTt cation is a better flyer than DMTt cation, because the sulphur-trityl bond is more easily cleaved than the corresponding oxygen-trityl bond, or because of a combination of the two, in all the spectra the peak corresponding to the TMTt cation was significantly bigger than the peak corresponding to the DMTt cation. The difference was still bigger in those spectra run without the assistance of matrix.

### Example 6

### Comparison of the Ionisability of Trityl Ethers and Trityl Thioethers

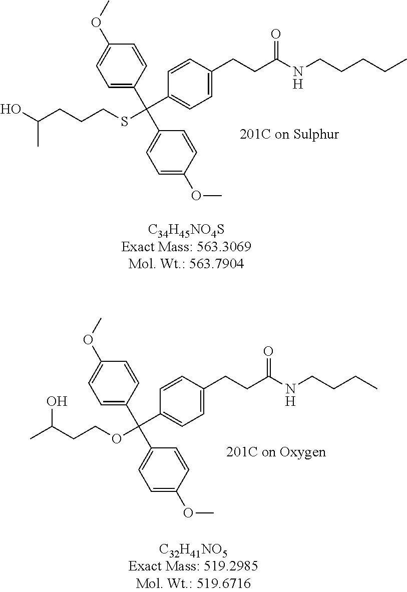

The following tags were prepared and were purified via preparative TLC and filtered using the Millex syringe driven filter to ensure no fine particles of silica were present.

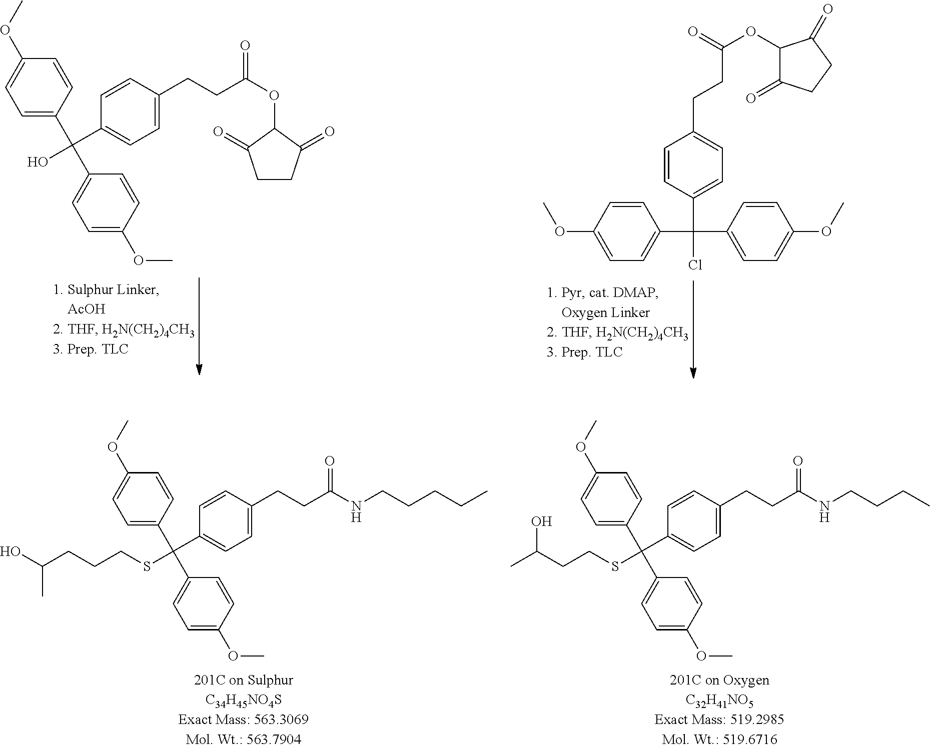

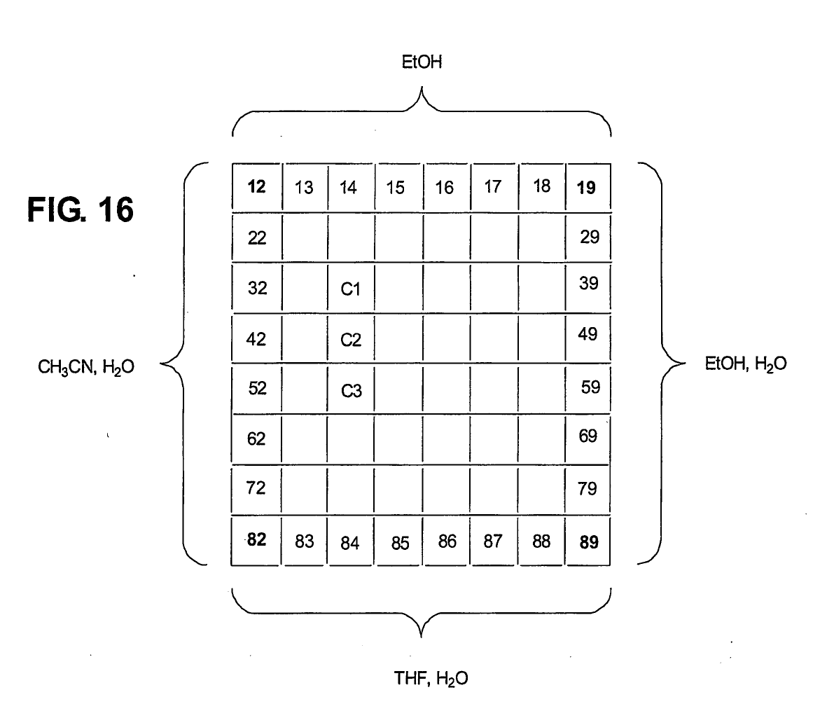

Equimolar solutions of both tags were then prepared and spotted on to a gold plate with 64 wells as shown in FIG. 16. The trityl tag-mix (equimolar solution of both trityl tags) was spotted on wells C1 with matrix and C2 without matrix.

The samples were spotted and the plate allowed to dry. The plate was analyzed by MALDI-TOF using the Voyager Spec (ABI) with 5-dihydroxybenzoic acid (DHB) matrix and without DHB matrix.

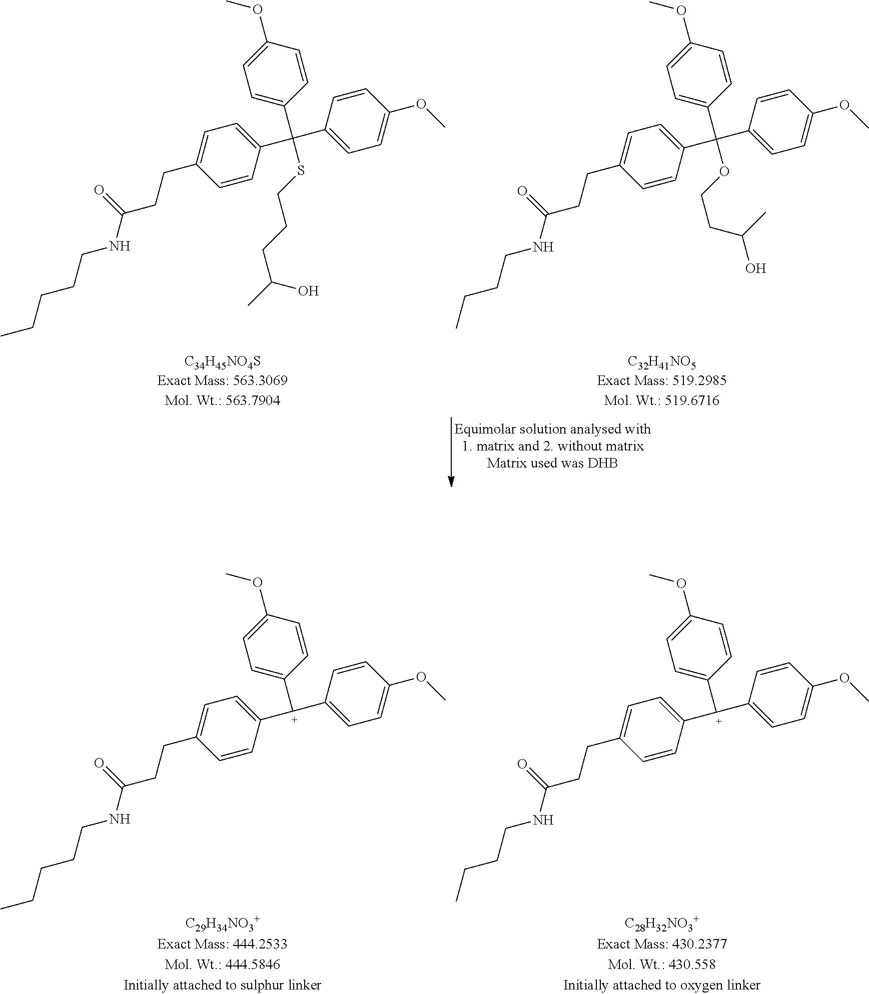

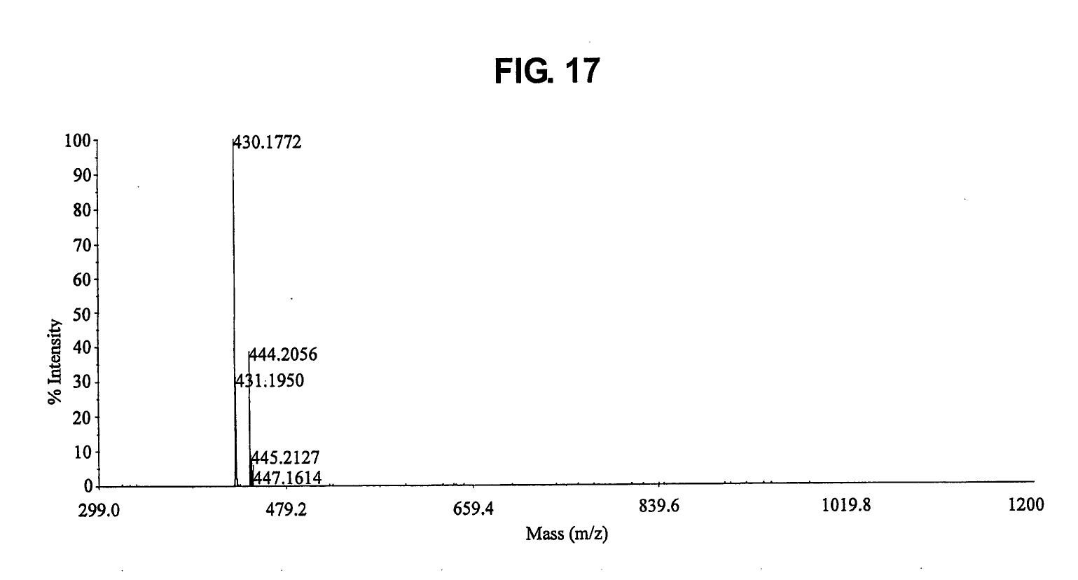

Trityl ethers are more easily observed in the presence of matrix. The acidic nature of DHB results in an initial cleavage of the trityl ether bond, therefore the trityl is already present in the form of a trityl cation, whereas the trityl thioether is resistant to DHB and is not cleaved (well C1). The spectrum shown in FIG. 17 confirmed that the 201C tag on oxygen was the more intense peak (well C1).

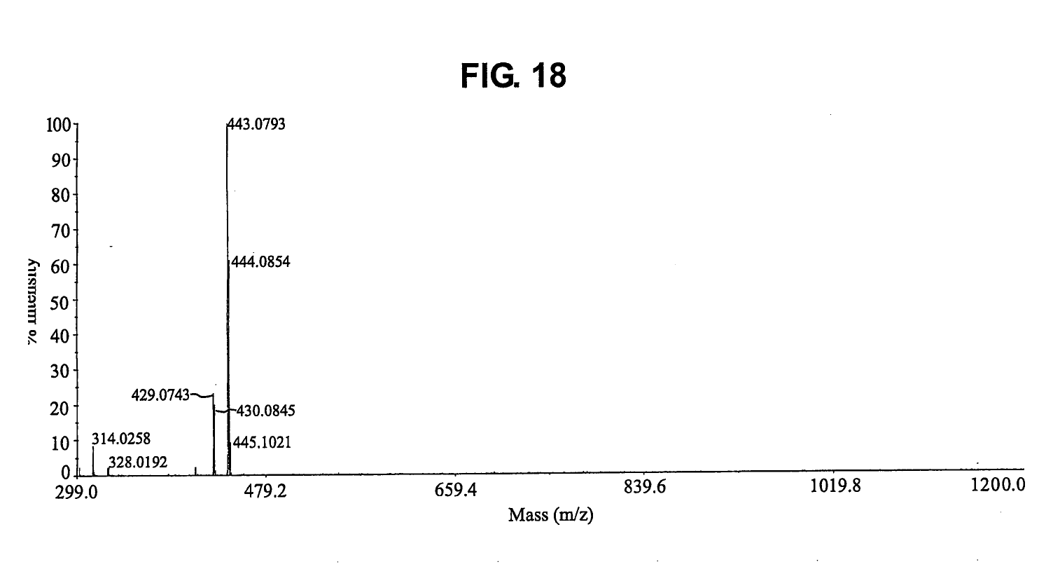

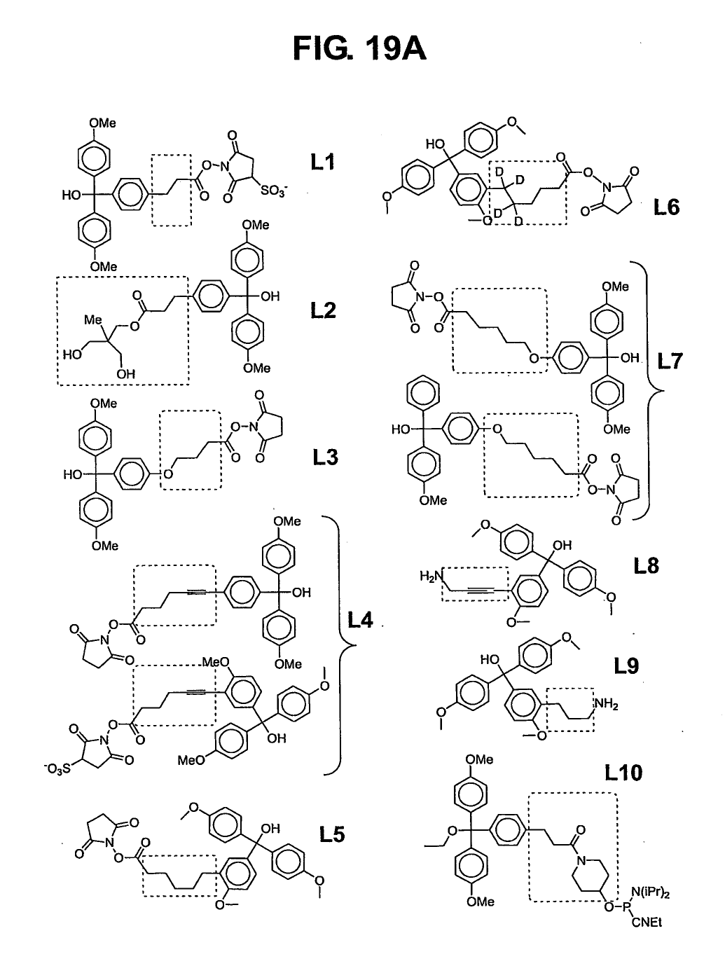

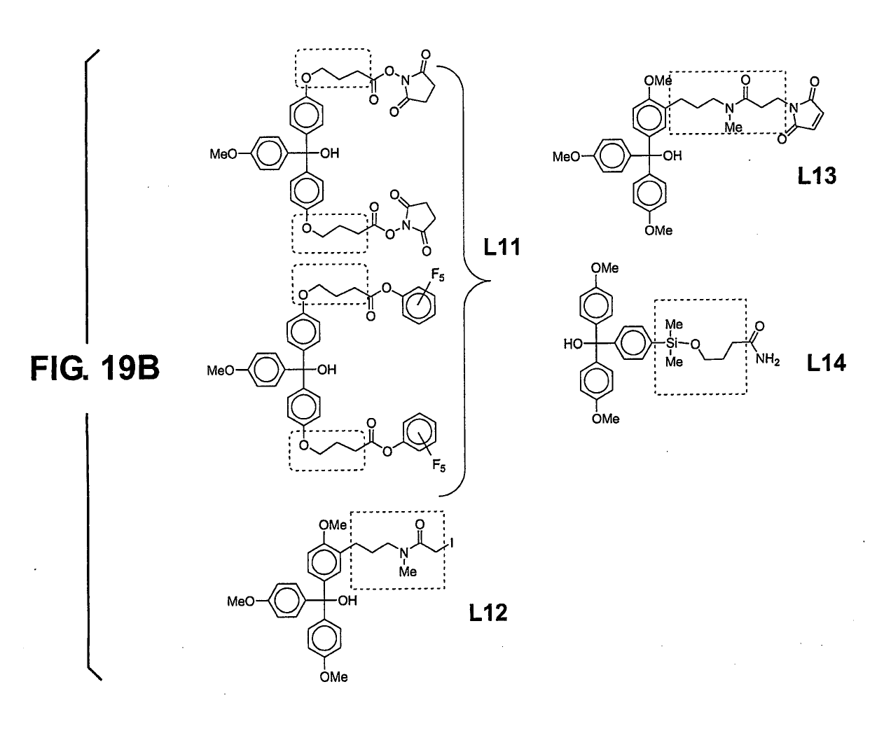

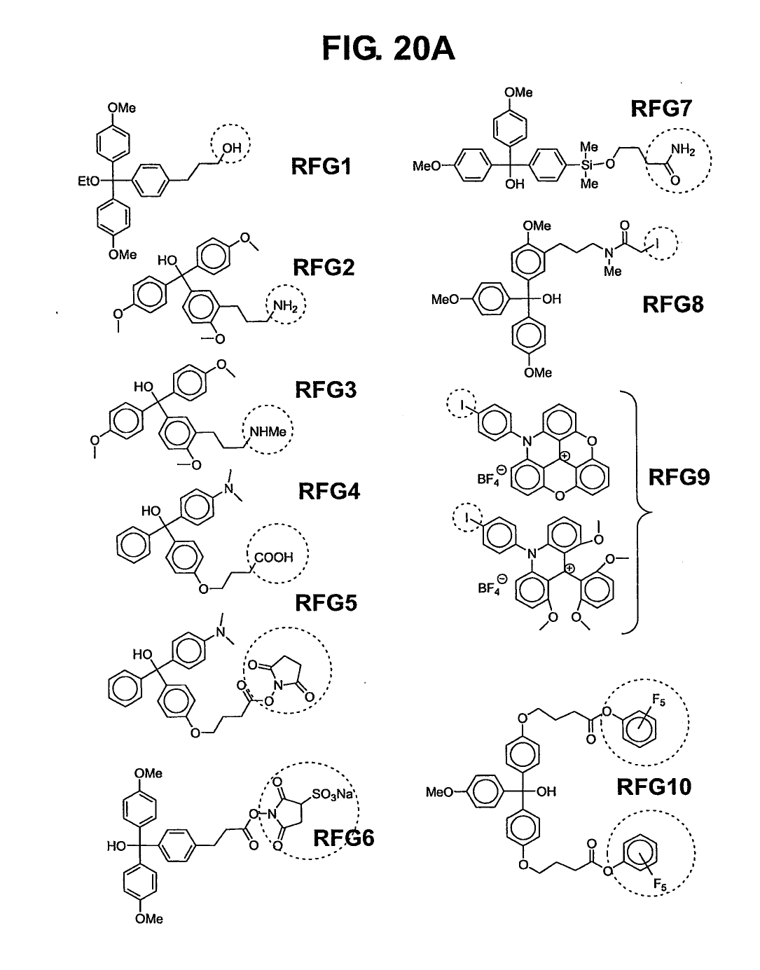

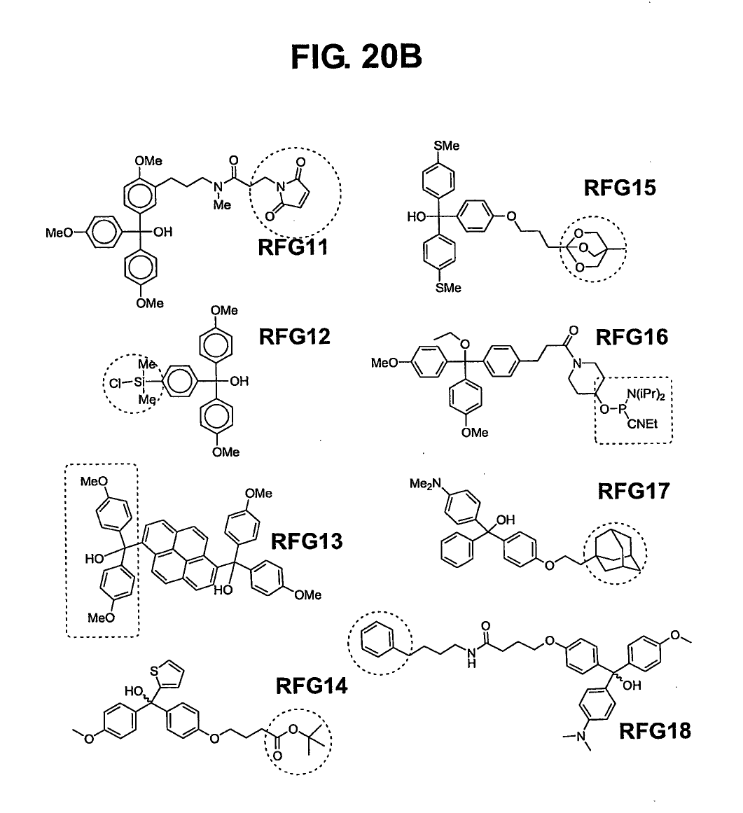

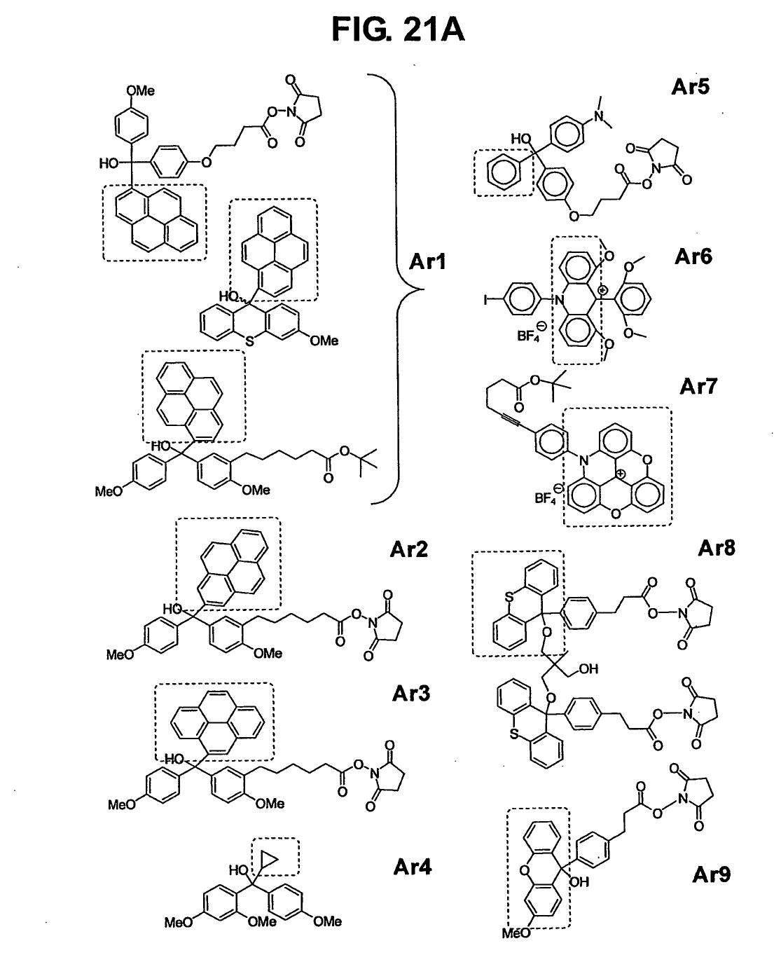

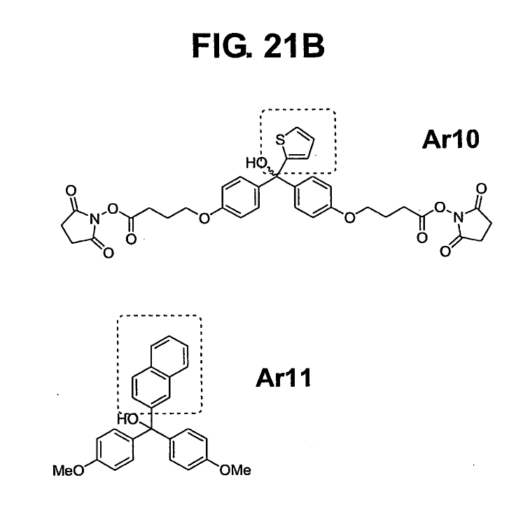

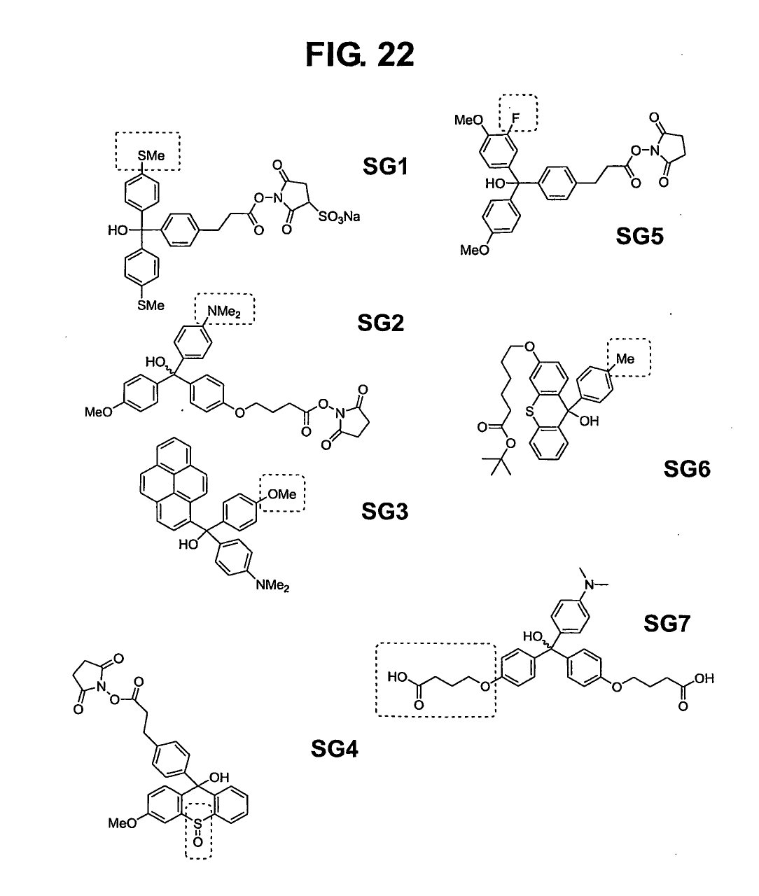

By removing matrix and analysing both the trityl ether and trityl thioether, it was observed that 201C on sulphur was more readily cleaved than the trityl ether (well C2). This is seen in the spectrum in FIG. 18.

It will be understood that the invention is described above by way of example only and modifications may be made whilst remaining within the scope and spirit of the invention.

1. A method of forming an ion of formula (I): 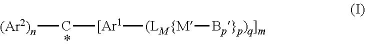
comprising the steps of:
(i) reacting a compound of the formula (IIa): 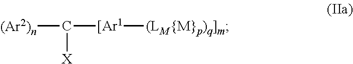
with a biopolymer, B_{P}, having at least one group capable of reacting with M to form a covalent linkage, to provide a biopolymer derivative of the formula (IIIa): 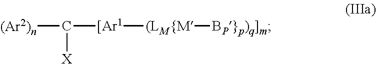
and
(ii) cleaving the C—X bond between X and the α-carbon atom of the derivative of formula (HIa) to form the ion of formula (I);
where:
C★ is a carbon atom bearing a single positive charge or a single negative charge;
X is a group comprising a thioether sulphur atom bound directly to the α-carbon which is capable of being cleaved from the α-carbon atom to form an ion of formula (I);
M is independently a group capable of reacting with B_{P} to form the covalent linkage;
B_{P}′ is independently the biopolymer residue of B_{P} produced on formation of the covalent linkage;
M′ is independently the residue of M produced on formation of the covalent linkage;
Ar¹ is independently an aromatic group or an aromatic group substituted with one or more A;
Ar² is independently an aromatic group or an aromatic group substituted with one or more A;
optionally wherein (a) two or three of the groups Ar¹ and Ar² are linked together by one or more L⁵, where L⁵ is independently a single bond or a linker atom or group; and/or (b) two, or three of the groups Ar¹ and Ar² together form an aromatic group or an aromatic group substituted with one or more A;
A is independently a substituent;
L_{M} is independently a single bond or a linker atom or group;
n=0, 1 or 2 and m=1, 2, or 3, provided the sum of n+m=3;
p independently=1 or more; and
q independently=1 or more.

2. A biopolymer derivative of the formula (IIIa).

3. A compound of the formula (IIa).

4. A method of forming an ion of formula (I) comprising the steps of:
(i) reacting a solid support of formula (IVai): 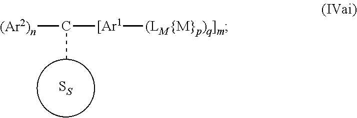
with a biopolymer, B_{P}, having at least one group capable of reacting with M to form a covalent linkage, to provide a modified solid support of the formula (Vai): 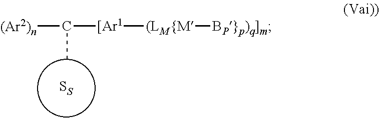
and
(iia) cleaving the C—S_{S} bond between the α-carbon atom of the modified solid support of formula (Vai) and the solid support S_{S} to form the ion of formula (I);
where:
Ar¹, Ar², B_{P}′, L_{M}, M, M′, n, m, p and q are as defined in claim 1;
S_{S} is a solid support; and
C—S_{S} comprises a cleavable bond between C and S_{S} involving a thioether sulphur atom bound directly to the α-carbon atom.

5. A method of forming an ion of formula (I) comprising the steps of:
(i) reacting a solid support of formula (IVaii), or (IVaiii): 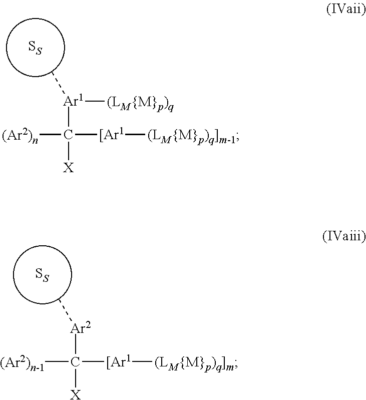
with a biopolymer, B_{P}, having at least one group capable of reacting with M to form a covalent linkage, to provide a modified solid support of the formula (Vaii), or (Vaiii), respectively: 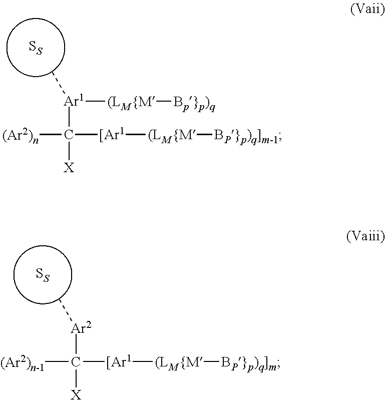
and either:
(iib) for modified solid supports of formula (Vaii), either simultaneously or sequentially, cleaving the C—X bond between X and the α-carbon atom and cleaving the S_{S}—Ar¹ bond between the solid support and the Ar¹ group to form the ion of formula (I); or
(iic) for modified solid supports of formula (Vaiii), either simultaneously or sequentially, cleaving the C—X bond between X and the α-carbon atom and cleaving the S_{S}—Ar² bond between the solid support and the Ar² group to form the ion of formula (I);
where:
X, Ar¹, Ar², B_{P}′, M, M′, n, m, p and q are as defined in claim 1;
S_{S} is a solid support;
S_{S}—Ar¹ comprises a cleavable bond between Ar¹ and S_{S}; and
S_{S}—Ar² comprises a cleavable bond between Ar² and S_{S}.

6. A method of forming an ion of formula (I) comprising the steps of:
(i) reacting a solid support of formula (IVaiv): 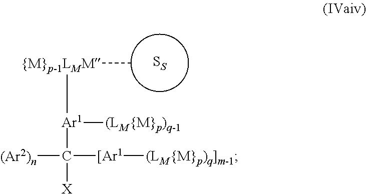
with a biopolymer, B_{P}, having at least one group capable of reacting with M to form a covalent linkage, to provide a modified solid support of the formula (Vaiv): 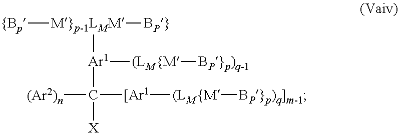
and
(iia) cleaving the C—X bond between X and the α-carbon atom to form the ion of formula (I); or
where:
X, Ar¹, Ar², B_{P}′, L_{M}, M, M′, p, q, n, in, and S_{S} are as defined in claims 8 and 9;
M″—S_{S} comprises a bond between M″ and S_{S}; and
M″ is the same as M except that S_{S} is bound to a portion of M which does not form part of M′.

7. A solid support of the formula (IVai), (IVaii), (IVaiii) or (IVaiv).

8. A modified solid support of the formula (Vai), (Vaii), (Vaiii) or (Vaiv).

9. A method of claim 5 or 6 or a product of claim 7 or 8 wherein the biopolymer is a synthetic biopolymer.

10. A method or product of claim 9 wherein the synthetic biopolymer is an oligonucleotide, a peptide or a carbohydrate.

11. A method for analysing a biopolymer, B_{P}, comprising the steps of
(i) reacting the biopolymer B_{P} with a compound of formula (IIa) or a solid support of formula (IVai), (IVaii), (IVaiii) or (IVaiv);
(ii) providing an ion of formula (I); and
(iii) analysing the ion of formula (I) by mass spectrometry.

12. In a method for analysing a biopolymer, B_{P}, the improvement consisting of: (i) reacting a biopolymer, B_{P} with a compound of formula (IIa) or a solid support of formula (IVai), (IVaii), (IVaiii) or (IVaiv); (ii) providing an ion of formula (I); and (iii) analysing the ion by mass spectrometry.

13. A method of claim 11 or claim 12 wherein the analysis by mass spectrometry is carried out in a spectrometer which is suitable for MALDI-TOF spectrometry.

14. A method of any of claim 1, 5, 6 or 9-13 or a product of any of claim 2, 3, 7 or 8, wherein C★ bears a single positive charge, such that the ion of formula (I) has the structure:

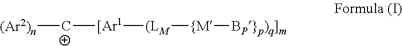

15. A method of any of claim 1, 5, 6 or 9-14 or a product of any of claim 2, 3, 7, 8 or 14 wherein n=2 and m=1.

16. A method of any of claim 1, 5, 6 or 9-15 or a product of any of claim 2, 3, 7, 8, 14 or 15 wherein p=1, 2 or 3.

17. A method of any of claim 1, 5, 6 or 9-16 or a product of any of claim 2, 3, 7, 8 or 14-16 wherein p=1.

18. A method of any of claim 1, 5, 6 or 9-17 or a product of any of claim 2, 3, 7, 8 or 14-17 wherein q=1, 2 or 3.

19. A method of any of claim 1, 5, 6 or 9-18 or a product of any of claim 2, 3, 7, 8 or 14-18 wherein q=1.

20. A method of any of claim 1, 5, 6 or 9-19 or a product of any of claim 2, 3, 7, 8 or 14-19 wherein n=2, m=1, p=1 and q=1, such that the ion of formula (I) has the structure:

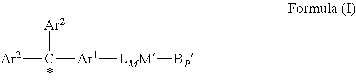

21. A method of any of claim 1, 5, 6 or 9-20 or a product of any of claim 2, 3, 7, 8 or 14-20 wherein the biopolymer is a polymer found in biological samples.

22. A method or product of claim 21 wherein the biopolymer is a polypeptide, polysaccharide, or polynucleotide.

23. A method or product of claim 22 wherein the biopolymer is a polypeptide.

24. A method or product of any of claims 21-23 wherein the biopolymer does not readily form a molecular ion on illumination of laser light at 340 nm.

25. A method of any of claim 1, 5, 6 or 9-24 or a product of any of claim 2, 3, 7, 8 or 14-24 wherein the ratio m(B_{P}′)/m(IX) is more than 2, where m(IX) is the mass of the fragment (IX) 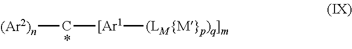
of the cation of formula (I) and m(B_{P}′) is the mass of the biopolymer residue B_{P}′.

26. A method of any of claim 1, 5, 6 or 9-25 or a product of any of claim 2, 3, 7, 8 or 14-25 wherein M is: —NR₂; —SR; —OR; —B(R)Y; —BY₂; —C(R)₂Y; —C(R)Y₂; —CY₃; —C(═Z)Y; —Z—C(═Z)Y; —C(═Z)R; —C(R)(OH)OR; —C(R)(OR)₂; —S(═O)Y; —Z—S(═O)Y; —S(═O)₂Y; —Z—S(═O)₂Y; —S(═O)₃Y; —Z—S(═O)₃Y; —P(═Z)(ZR)Y; —P(═Z)Y₂; —Z—P(═Z)(ZR)Y; —Z—P(═Z)Y₂; —P(═Z)(R)Y; —Z—P(═Z)(R)Y; or —N═C(═Z), where Y is independently a leaving group, Z is independently O, S or N(R) and R is independently H, C₁₋₈hydrocarbyl or C₁₋₈hydrocarbyl substituted with one or more A.

27. A method of any of claim 1, 5, 6 or 9-25 or a product of any of claim 2, 3, 7, 8 or 14-25 wherein M is: —N(R)—; —S—; —O—; —B(Y)—; —C(R)(Y)—; —CY₂—; —C(═O)—; —C(OH)(OR)—; or —C(OR)₂—, where Y is independently a leaving group and R is independently H, C₁₋₈hydrocarbyl or C₁₋₈hydrocarbyl substituted with one or more A.

28. A method of any of claim 1, 5, 6 or 9-25 or a product of any of claim 2, 3, 7, 8 or 14-25 wherein M is: 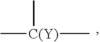
where Y is a leaving group.

29. A method of any of claim 1, 5, 6 or 9-25 or a product of any of claim 2, 3, 7, 8 or 14-25 wherein the covalent linkage is selected from those produced through the reaction of one the following groups: —CO—NH—; biotin-(strept)avidin; 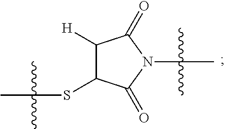
or —NH—CS—NH—.

30. A method of any of claim 1, 5, 6 or 9-29 or a product of any of claim 2, 3, 7, 8 or 14-29 wherein L_{M} is O or S.

31. A method of any of claim 1, 5, 6 or 9-29 or a product of any of claim 2, 3, 7, 8 or 14-29 wherein L_{M} is -E^{M}-, -(D^{M})ₜ-, -(E^{M}-D^{M})ₜ, -(D^{M}-E^{M})ₜ-, -E^{M}-(D^{M}-E^{M})ₜ- or -D^{M}-(E^{M}-D^{M})ₜ- (in the orientation Ar¹-(L_{M}{M}ₚ)_{q} or Ar¹-(L_{M}{M′}ₚ)_{q}, as appropriate),
where:
a sufficient number of linking covalent bonds, in addition to the covalent bonds at the chain termini shown, are provided on groups E^{M} and D^{M} for linking the p instances of M (or M′) groups;
D^{M} is independently C₁₋₈hydrocarbylene or C₁₋₈hydrocarbylene substituted with one or more A;
E^{M} (in the orientation Ar¹-(L_{M}{M}ₚ)_{q} or Ar¹-(L_{M}{M′}ₚ)_{q}, as appropriate) is independently —Z^{M}—, —C(═Z^{M})—, —Z^{M}C(═Z^{M})—, —C(═Z^{M})Z^{M}—, —Z^{M}C(═Z^{M})Z^{M}—, —S(═O)—, —Z^{M}S(═O)—, —S(═O)Z^{M}—, —Z^{M}S(═O)Z^{M}—, —S(═O)₂—, —Z^{M}S(═O)₂—, —S(D)₂Z^{M}—, —Z^{M}S(═O)₂Z^{M}—, where Z^{M} is independently O, S or N(R^{M}) and where R^{M} is independently H, C₁₋₈hydrocarbyl (e.g. C₁₋₈alkyl) or C₁₋₈hydrocarbyl substituted with one or more A; and
t=1 or more.

32. A method of any of claim 1, 5, 6 or 9-31 or a product of any of claim 2, 3, 7, 8 or 14-31 wherein the group X is sulfanyl, hydrocarbylsufanyl, hydrocarbylsufanyl substituted with one or more A, heterohydrocarbylsufanyl, or heterohydrocarbylsufanyl substituted with one or more A.

33. A method of any of claim 1, 5, 6 or 9-32 or a product of any of claim 2, 3, 7, 8 or 14-32 wherein Ar² is independently cyclopropyl, cyclopropyl substituted with one or more A, aryl, aryl substituted with one or more A, heteroaryl, or heteroaryl substituted with one or more A.

34. A method of any of claim 1, 5, 6 or 9-33 or a product of any of claim 2, 3, 7, 8 or 14-33 wherein Ar² is

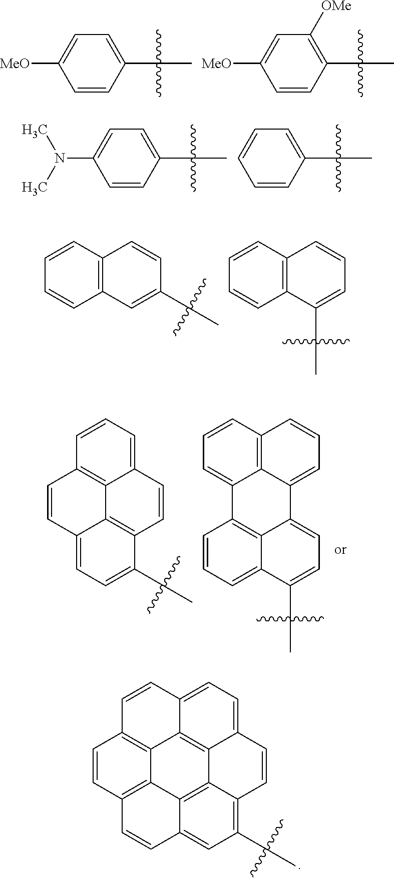

35. A method of any of claim 1, 5, 6 or 9-34 or a product of any of claim 2, 3, 7, 8 or 14-34 wherein Ar¹ is independently cyclopropylene, cyclopropylene substituted with one or more A, arylene, arylene substituted with one or more A, heteroarylene, or heteroarylene substituted with one or more A.

36. A method of any of claim 1, 5, 6 or 9-35 or a product of any of claim 2, 3, 7, 8 or 14-35 wherein Ar¹ is

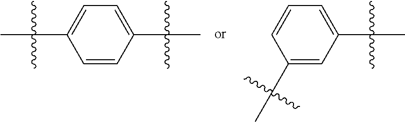

37. A method of any of claim 1, 5, 6 or 9-36 or a product of any of claim 2, 3, 7, 8 or 14-36 wherein L⁵ is O or S.

38. A method of any of claim 1, 5, 6 or 9-36 or a product of any of claim 2, 3, 7, 8 or 14-36 wherein L⁵ is -E⁵-, -(D⁵)_{t′}-, -(E⁵-D⁵)_{t′}-, -(D⁵-E⁵)_{t′}- or -D⁵-(E⁵-D⁵)_{t′}-,
where:
D⁵ is independently C₁₋₈hydrocarbylene or C₁₋₈hydrocarbylene substituted with one or more A;
E⁵ is independently —Z⁵—, —C(═Z⁵)—, —Z⁵C(═Z⁵)—, —C(═Z⁵)Z⁵—, —Z⁵C(═Z⁵)Z⁵—, —S(═O)—, —Z⁵S(═O)—, —S(═O)Z⁵—, —Z⁵S(═O)Z⁵—, —S(═O)₂—, —Z⁵S(═O)₂—, —S(═O)₂Z⁵—, —Z⁵S(═O)₂Z⁵—, where Z⁵ is independently O, S or N(R⁵) and where R⁵ is independently H, C₁₋₈hydrocarbyl or C₁₋₈hydrocarbyl substituted with one or more A; and
t′=1 or more.

39. A method of any of claim 1, 5, 6 or 9-38 wherein the step of cleaving the C—X bond or C—S_{S} bond is carried out in the absence of an acidic matrix.

40. A method of claim 39 wherein all steps are carried out in the absence of an acidic matrix.
